(19) United States
(12) Patent Application Publication
(10) Pub. No.: US 2026/0085082 A1
(43) Pub. Date: Mar. 26, 2026

(54) KDM1A INHIBITORS FOR THE TREATMENT OF DISEASE

(71) Applicant: Imago Biosciences, Inc., San Carlos, CA (US)

(72) Inventors: Hugh Y. Rienhoff, Jr., San Carlos, CA (US); Cassandra Celatka, Hull, MA (US); Amy E. Tapper, Boston, MA (US); Nivedita Namdev, Westford, MA (US); Michael Clare, Skokie, IL (US)

(73) Assignee: Imago Biosciences, Inc., San Carlos, CA (US)

(21) Appl. No.: 19/137,840

(22) PCT Filed: Dec. 15, 2023
(86) PCT No.: PCT/US2023/084162

§ 371 Date: Jun. 11, 2025

Related U.S. Application Data
(60) Provisional application No. 63/476,990, filed on Dec. 23, 2022.

(51) Int. Cl. C07F 9/6558; A61K 31/4155; A61K 31/454; A61K 31/4545; A61K 31/496; A61K 31/541; A61K 31/675; A61K 31/706; A61P 35/02; C07D 231/40; C07D 401/06; C07D 401/12; C07D 401/14; C07D 403/06; C07D 403/10; C07D 403/12; C07D 405/14; C07D 413/14; C07D 417/14; C07D 491/056

(52) U.S. Cl. CPC C07F 9/65583; A61K 31/4155; A61K 31/454; A61K 31/4545; A61K 31/496; A61K 31/541; A61K 31/675; A61K 31/706; A61P 35/02; C07D 231/40; C07D 401/06; C07D 401/12; C07D 401/14; C07D 403/06; C07D 403/10; C07D 403/12; C07D 405/14; C07D 413/14; C07D 417/14; C07D 491/056

(57) ABSTRACT

Disclosed herein are new compounds and compositions and their application as pharmaceuticals for the treatment of diseases. Methods of inhibition of KDM1A, methods of increasing gamma globin gene expression, and methods to induce differentiation of cancer cells in a human or animal subject are also provided for the treatment of diseases such as acute myelogenous leukemia.

### CROSS-REFERENCE TO RELATED APPLICATIONS

This international application claims the benefit of priority to U.S. Provisional Application No. 63/476,990, filed Dec. 23, 2022, the entirety of which is incorporated herein by reference.

### FIELD OF THE INVENTION

Inhibiting the enzyme KDM1A (also known as lysine-specific demethylase 1, LSD1, Flavin-containing Amine Oxidase Domain-Containing Protein, AOF2, BRAF35-HDAC Complex Protein BHC110, FAD-Binding Protein BRAF35-HDAC Complex) would be useful for the treatment of diseases such as cancer and heritable diseases such as Wilson disease, cardiomyopathies, and hemoglobinopathies.

Provided are new compounds, compositions, and methods for treating diseases associated with KDM1A activity.

### SUMMARY

Provided herein is a compound of Formula I:

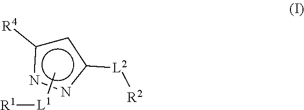

or a salt or tautomer thereof, wherein:
L¹ is chosen from a bond and —(CH₂)ₖ—;
L² is chosen from —C(O)NH—, —NHC(O)—, and —C(O)—;
R¹ is chosen from phenyl, 5-membered monocyclic heteroaryl, and 10-membered bicyclic heteroaryl, either of which is substituted with one R^{5a} and one or more R^{5b};
R² is chosen from cyclohexyl, azetidinyl, pyrrolidinyl, and piperidinyl, any of which is optionally substituted with one or more R⁶;
k is chosen from 1, 2, and 3;
R⁴ is phenyl substituted with one R^{7a} and one or more R^{7b};
R^{5a} is chosen from CH₃, CH₂CH₃, OCH₃, 5- to 6-membered heterocycloalkyl, and 5-membered monocyclic heteroaryl, and 10-membered bicyclic heteroaryl, wherein the heterocycloalkyl and heteroaryl are optionally substituted by one or more R⁹;
each R^{5b} is independently chosen from H, halo, and OCH₃;
each R⁶ is independently chosen from —NHR⁸, —NHC(NH)NHR⁸, and —CH₂NHR⁸;
R^{7a} is cyano;
each R^{7b} is independently chosen from halo;
each R⁸ is independently chosen from H and C₁₋₃ alkyl; and
R⁹ is chosen from cyano, C₁₋₃ alkyl, C₁₋₃ alkoxy, hydroxy, phosphonate, oxo, or any combination thereof,
wherein if R^{5a} is

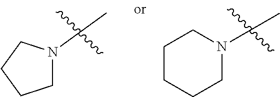 [SMILES: *N1CCCC1 ; *N1CCCCC1]

optionally substituted by one or more R⁹, each R^{5b} is H.

Also provided herein is a compound of Formula I or II:

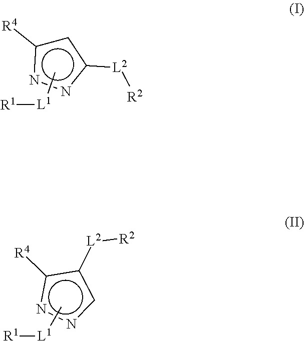

or a salt or tautomer thereof, wherein:
L¹ is a bond;
L² is chosen from —C(O)NH—, —NHC(O)—, and —C(O)—;
R¹ is phenyl substituted with R^{5a};
R² is chosen from cyclohexyl, azetidinyl, pyrrolidinyl, and piperidinyl, any of which is optionally substituted with one or more R⁶;
R⁴ is phenyl which is substituted with one R^{7a} and optionally substituted with one or more R^{7b};
R^{5a} is OCH₃;
each R⁶ is independently chosen from —NHR⁸, —NHC(NH)NHR⁸, and —CH₂NHR⁸;
R^{7a} is cyano;
each R^{7b} is independently chosen from halo; and
each R⁸ is independently chosen from H and C₁₋₃ alkyl.

Also provided herein is a pharmaceutical composition comprising a compound as recited herein, or a salt or tautomer thereof, together with a pharmaceutically acceptable carrier.

Also provided herein is a method of inhibition of KDM1A comprising contacting KDM1A with a compound as recited herein, or a salt or tautomer thereof, or a pharmaceutical composition as recited herein.

Also provided herein is a method of treatment of a KDM1A-mediated disease comprising the administration of a therapeutically effective amount of a compound as recited herein, or a salt or tautomer thereof, or a pharmaceutical composition as recited herein, to a patient in need thereof.

Also provided herein is a method of treatment of a globin-mediated disease comprising the administration of a therapeutically effective amount of a compound as recited herein, or a salt or tautomer thereof, or a pharmaceutical composition as recited herein, to a patient in need thereof.

Also provided herein is a method for achieving an effect in a patient comprising the administration of a therapeutically effective amount of a compound as recited herein, or a salt or tautomer thereof, or a pharmaceutical composition as recited herein, to a patient, wherein the effect is chosen from an elevation of red blood cell count, an elevation of the red blood cell count of red cells containing fetal hemoglobin, an elevation in the total concentration of fetal hemoglobin in red cells, an elevation in the total concentration of fetal hemoglobin in reticulocytes, an increase in the transcription of the gamma globin gene in bone marrow-derived red cell precursors, e.g., pro-erythroblasts, a reduction in the number of sickle cell crises a patient experiences over a unit period of time, a halt to or prevention of tissue damage e.g., in the heart, spleen, brain or kidney caused by sickling cells, a reduction in the proportion of red cells that undergo sickling under physiological conditions of relative hypoxia as measured using patient blood in an in vitro assay, an increase in the amount of histone 3 lysine methylation at lysine position 4 (H3K4me1 and H3K4me2), and/or a decrease in the amount of histone 3 methylation at lysine position 9 (H3K9me1 or H3K4me2) near or at the gamma globin promoter as assayed by ChIP using cells derived from a treated patient.

Also provided herein is a method of inhibiting at least one KDM1A function comprising the step of contacting KDM1A with a compound as recited herein, or a salt or tautomer thereof, or a pharmaceutical composition as recited herein, wherein the inhibition is measured by phenotype of red cells or their precursors either cultured or in vivo in humans or mouse or transgenic mice containing the human beta globin locus or portions thereof, the ability of cancer cells to proliferate, become differentiated, or induced to undergo apoptosis, the expression of specific genes known to be regulated by KDM1A activity such as gamma globin or HOXA9, a change in the histone methylation states, a change in the methylation state of proteins known to be demethylated by KDM1A such as G9a, p53, DNMT1 or SUV39H1, expression of KDM1A-regulated genes, or binding of KDM1A with a natural binding partner such as CoREST, NuRD, DNMT1 or HDACs.

Also provided herein is a method of treatment of a bone marrow failure syndrome, comprising the administration of a therapeutically effective amount of a compound as recited herein, or a salt or tautomer thereof, or a pharmaceutical composition as recited herein, to a patient in need thereof.

Also provided herein is a method of treatment of a hemoglobinopathy, comprising the administration of a therapeutically effective amount of a compound as recited herein, or a salt or tautomer thereof, or a pharmaceutical composition as recited herein, to a patient in need thereof.

### DETAILED DESCRIPTION

Provided herein is a compound of Formula I:

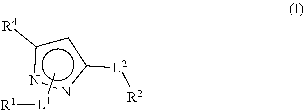

or a salt or tautomer thereof, wherein:
L¹ is chosen from a bond and —(CH₂)ₖ—;
L² is chosen from —C(O)NH—, —NHC(O)—, and —C(O)—;
R¹ is chosen from phenyl, 5-membered monocyclic heteroaryl, and 10-membered bicyclic heteroaryl, either of which is substituted with one R^{5a} and one or more R^{5b};
R² is chosen from cyclohexyl, azetidinyl, pyrrolidinyl, and piperidinyl, any of which is optionally substituted with one or more R⁶;
k is chosen from 1, 2, and 3;
R⁴ is phenyl substituted with one R^{7a} and one or more R^{7b};
R^{5a} is chosen from H, CH₃, CH₂CH₃, OCH₃, 5- to 6-membered heterocycloalkyl, and 5-membered monocyclic heteroaryl, and 10-membered bicyclic heteroaryl, wherein the heterocycloalkyl and heteroaryl are optionally substituted by one or more R⁹;
each R^{5b} is independently chosen from H, halo, and OCH₃;
each R⁶ is independently chosen from —NHR⁸, —NHC(NH)NHR⁸, and —CH₂NHR⁸;
R^{7a} is cyano;
each R^{7b} is independently chosen from halo;
each R⁸ is independently chosen from H and C₁₋₃ alkyl; and
R⁹ is chosen from cyano, C₁₋₃ alkyl, C₁₋₃ alkoxy, hydroxy, phosphonate, oxo, or any combination thereof,
wherein if R^{5a} is

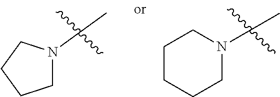 [SMILES: *N1CCCC1 ; *N1CCCCC1]

or optionally substituted by one or more R⁹, each R^{5b} is H.

In some embodiments, the compound of Formula I is a compound of Formula III:

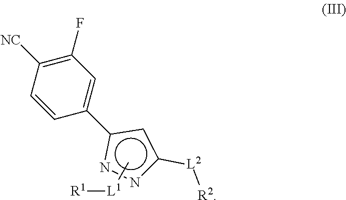

In some embodiments, the compound of Formula I is a compound of Formula IV:

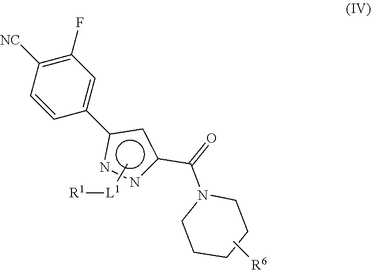

In some embodiments, L¹ is —(CH₂)ₖ—.

In some embodiments, k is 1 or 2.

In some embodiments, k is 1.

In some embodiments, k is 2.

In some embodiments, L¹ is a bond.

In some embodiments, the compound of Formula I is a compound of Formula V:

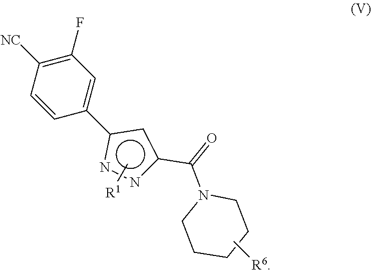

In some embodiments, R¹ is chosen from phenyl, 5-membered monocyclic heteroaryl, and 10-membered bicyclic heteroaryl, either of which is substituted with one R^{5a} and one or two R^{5b}.

In some embodiments, R¹ is phenyl substituted with R^{5a}.

In some embodiments, R^{5a} is chosen from CH₃, CH₂CH₃, OCH₃, 5- to 6-membered heterocycloalkyl, and 5-membered monocyclic heteroaryl, and 10-membered bicyclic heteroaryl, wherein the heterocycloalkyl and heteroaryl are optionally substituted with one, two, or three R⁹.

In some embodiments, R^{5a} is chosen from CH₃, CH₂CH₃, OCH₃, 5- to 6-membered heterocycloalkyl, and 5-membered monocyclic heteroaryl, and 10-membered bicyclic heteroaryl, wherein the heterocycloalkyl and heteroaryl are optionally substituted with two R⁹.

In some embodiments, R^{5a} is chosen from CH₃, CH₂CH₃, OCH₃, 5- to 6-membered heterocycloalkyl, and 5-membered monocyclic heteroaryl, and 10-membered bicyclic heteroaryl, wherein the heterocycloalkyl and heteroaryl are optionally substituted with one R⁹.

In some embodiments, R^{5a} is CH₃ or OCH₃.

In some embodiments, R^{5a} is OCH₃.

In some embodiments, R¹ is p-tolyl, 3-fluoro-4-methoxyphenyl, or 4-methoxyphenyl.

In some embodiments, R¹ is 4-methoxyphenyl.

In some embodiments, R¹ is 5-membered monocyclic heteroaryl substituted with R^{5a}.

In some embodiments, R¹ is isoxazol-5-yl substituted with R^{5a}.

In some embodiments, R^{5a} is CH₃ or CH₂CH₃.

In some embodiments, R¹ is 3-methylisoxazol-5-yl or 3-ethylisoxazol-5-yl.

In some embodiments, R^{5a} is chosen from pyrrolidin-1-yl, piperidin-1-yl, 4-oxopiperidin-1-yl, piperazin-1-yl, 3-oxopiperazin-1-yl, 1H-indazol-5-yl, 2H-indazol-5-yl, 1H-pyrazol-4-yl, and 1,1-dioxidothiomorpholin-4-yl, any of which is optionally substituted by one or more R⁹.

In some embodiments, R^{5a} is chosen from pyrrolidin-1-yl, piperidin-1-yl, 4-oxopiperidin-1-yl, piperazin-1-yl, 3-oxopiperazin-1-yl, 1H-indazol-5-yl, 2H-indazol-5-yl, 1H-pyrazol-4-yl, and 1,1-dioxidothiomorpholin-4-yl, any of which is optionally substituted by one, two, or three R⁹.

In some embodiments, R^{5a} is piperazin-1-yl which is optionally substituted by one or two R⁹.

In some embodiments, R^{5a} is piperazin-1-yl which is optionally substituted by one R⁹.

In some embodiments, R⁹ is C₁₋₃ alkyl.

In some embodiments, R⁹ is CH₃.

In some embodiments, R^{5a} is 4-methylpiperazin-1-yl.

In some embodiments, R^{5a} is piperidin-1-yl optionally substituted by one or two R⁹.

In some embodiments, R⁹ is chosen from methoxy, cyano, methyl, hydroxy,

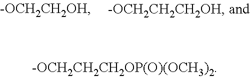

In some embodiments, L² is —C(O)NH— or —NHC(O)—.

In some embodiments, R² is chosen from cyclohexyl, azetidinyl, pyrrolidinyl, and piperidinyl, any of which is optionally substituted with one, two, or three R⁶.

In some embodiments, R² is chosen from cyclohexyl, azetidinyl, pyrrolidin-3-yl, piperidin-3-yl, and piperidin-4-yl, any of which is optionally substituted with R⁶.

In some embodiments, R² is cyclohexyl optionally substituted with R⁶.

In some embodiments, R⁶ is —NHR⁸.

In some embodiments, R⁸ is H.

In some embodiments, R² is 3-aminocyclohexyl.

In some embodiments, R² is pyrrolidin-3-yl, optionally substituted with R⁶.

In some embodiments, R² is pyrrolidin-3-yl.

In some embodiments, L² is —C(O)—.

In some embodiments, R² is chosen from azetidinyl and piperidin-1-yl, either of which is optionally substituted with R⁶.

In some embodiments, R² is piperidin-1-yl optionally substituted with R⁶.

In some embodiments, R² is 3-aminopiperidin-1-yl.

In some embodiments, R² is azetidin-1-yl optionally substituted with R⁶.

In some embodiments, R⁶ is chosen from —CH₂NHR⁸ and —NHR⁸.

In some embodiments, R⁴ is phenyl substituted with one R^{7a} and one, two, or three R^{7b}.

In some embodiments, R⁴ is phenyl substituted with one R^{7a} and substituted with one R^{7b}.

In some embodiments, R⁴ is 3-fluoro-4-cyanophenyl or 4-cyanophenyl.

In some embodiments, R⁴ is 3-fluoro-4-cyanophenyl.

In some embodiments, the compound of Formula I is chosen from:

| | |
|---|---|
| 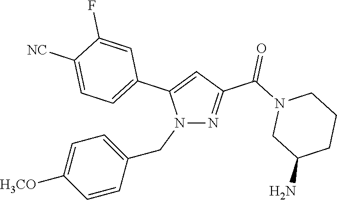 [SMILES: COc1ccc(Cn2nc(C(=O)N3CCC[C@@H](N)C3)cc2-c2ccc(C#N)c(F)c2)cc1] | (R)-4-(3-(3-aminopiperidine-1-carbonyl)-1- (4-methoxybenzyl)-1H-pyrazol-5-yl)-2- fluorobenzonitrile; |
| 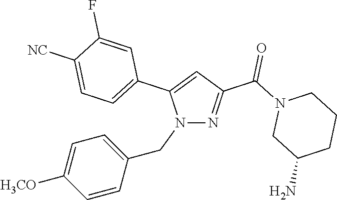 [SMILES: COc1ccc(Cn2nc(C(=O)N3CCC[C@H](N)C3)cc2-c2ccc(C#N)c(F)c2)cc1] | (S)-4-(3-(3-aminopiperidine-1-carbonyl)-1-(4- methoxybenzyl)-1H-pyrazol-5-yl)-2-fluoro- benzonitrile; |
| 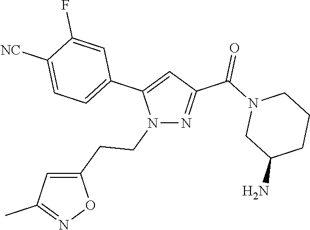 [SMILES: Cc1cc(CCn2nc(C(=O)N3CCC[C@@H](N)C3)cc2-c2ccc(C#N)c(F)c2)on1] | (R)-4-(3-(3-aminopiperidine-1-carbonyl)-1- (2-(3-methylisoxazol-5-yl)ethyl)-1H-pyrazol- 5-yl)-2-fluorobenzonitrile; |
| 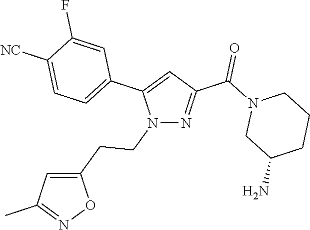 [SMILES: Cc1cc(CCn2nc(C(=O)N3CCC[C@H](N)C3)cc2-c2ccc(C#N)c(F)c2)on1] | (S)-4-(3-(3-aminopiperidine-1-carbonyl)-1-(2- (3-methylisoxazol-5-yl)ethyl)-1H-pyrazol-5- yl)-2-fluorobenzonitrile; |
| 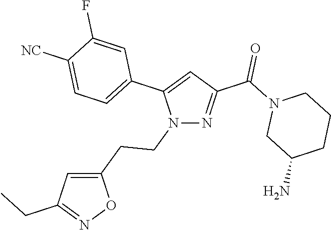 [SMILES: CCc1cc(CCn2nc(C(=O)N3CCC[C@H](N)C3)cc2-c2ccc(C#N)c(F)c2)on1] | (S)-4-(3-(3-aminopiperidine-1-carbonyl)-1-(2- (3-ethylisoxazol-5-yl)ethyl)-1H-pyrazol-5-yl)- 2-fluorobenzonitrile; |
| 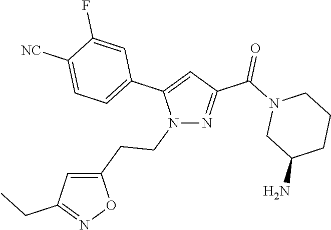 [SMILES: CCc1cc(CCn2nc(C(=O)N3CCC[C@@H](N)C3)cc2-c2ccc(C#N)c(F)c2)on1] | (R)-4-(3-(3-aminopiperidine-1-carbonyl)-1- (2-(3-ethylisoxazol-5-yl)ethyl)-1H-pyrazol-5- yl)-2-fluorobenzonitrile; |
| 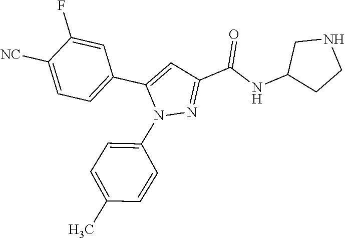 [SMILES: Cc1ccc(-n2nc(C(=O)NC3CCNC3)cc2-c2ccc(C#N)c(F)c2)cc1] | 5-(4-cyano-3-fluorophenyl)-N-(pyrrolidin-3- yl)-1-(p-tolyl)-1H-pyrazole-3-carboxamide; |
| 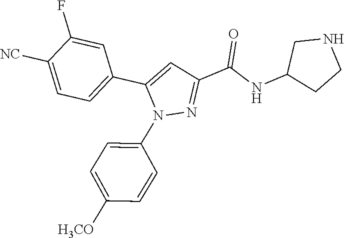 [SMILES: COc1ccc(-n2nc(C(=O)NC3CCNC3)cc2-c2ccc(C#N)c(F)c2)cc1] | 5-(4-cyano-3-fluorophenyl)-1-(4- methoxyphenyl)-N-(pyrrolidin-3-yl)-1H- pyrazole-3-carboxamide; |
| 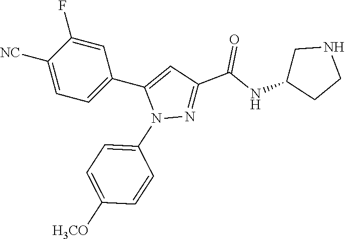 [SMILES: COc1ccc(-n2nc(C(=O)N[C@H]3CCNC3)cc2-c2ccc(C#N)c(F)c2)cc1] | (S)-5-(4-cyano-3-fluorophenyl)-1-(4- methoxyphenyl)-N-(pyrrolidin-3-yl)-1H- pyrazole-3-carboxamide; |
| 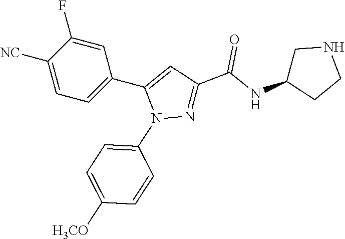 [SMILES: COc1ccc(-n2nc(C(=O)N[C@@H]3CCNC3)cc2-c2ccc(C#N)c(F)c2)cc1] | (R)-5-(4-cyano-3-fluorophenyl)-1-(4- methoxyphenyl)-N-(pyrrolidin-3-yl)-1H- pyrazole-3-carboxamide; |
| 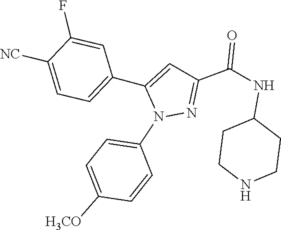 [SMILES: COc1ccc(-n2nc(C(=O)NC3CCNCC3)cc2-c2ccc(C#N)c(F)c2)cc1] | 5-(4-cyano-3-fluorophenyl)-1-(4- methoxyphenyl)-N-(piperidin-4-yl)-1H- pyrazole-3-carboxamide; |
| 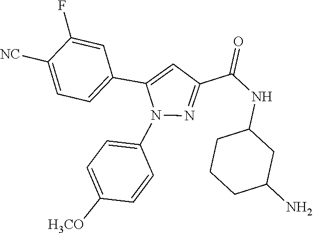 [SMILES: COc1ccc(-n2nc(C(=O)NC3CCCC(N)C3)cc2-c2ccc(C#N)c(F)c2)cc1] | N-(3-aminocyclohexyl)-5-(4-cyano-3- fluorophenyl)-1-(4-methoxyphenyl)-1H- pyrazole-3-carboxamide; |
| 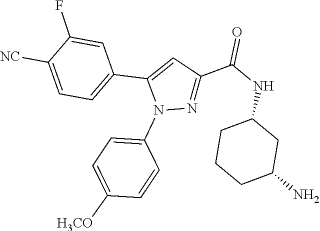 [SMILES: COc1ccc(-n2nc(C(=O)N[C@H]3CCC[C@@H](N)C3)cc2-c2ccc(C#N)c(F)c2)cc1] | N-((1S,3R)-3-aminocyclohexyl)-5-(4-cyano- 3-fluorophenyl)-1-(4-methoxyphenyl)-1H- pyrazole-3-carboxamide; |
| 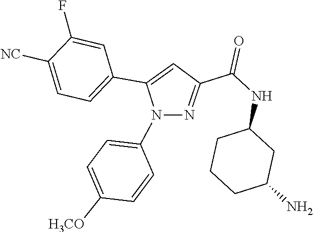 [SMILES: COc1ccc(-n2nc(C(=O)N[C@@H]3CCC[C@@H](N)C3)cc2-c2ccc(C#N)c(F)c2)cc1] | N-((1R,3R)-3-aminocyclohexyl)-5-(4-cyano- 3-fluorophenyl)-1-(4-methoxyphenyl)-1H- pyrazole-3-carboxamide; |
| 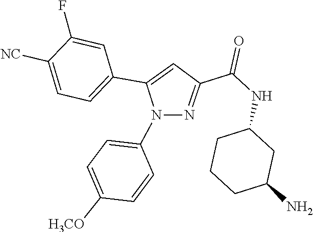 [SMILES: COc1ccc(-n2nc(C(=O)N[C@H]3CCC[C@H](N)C3)cc2-c2ccc(C#N)c(F)c2)cc1] | N-((1S,3S)-3-aminocyclohexyl)-5-(4-cyano-3- fluorophenyl)-1-(4-methoxyphenyl)-1H- pyrazole-3-carboxamide; |
| 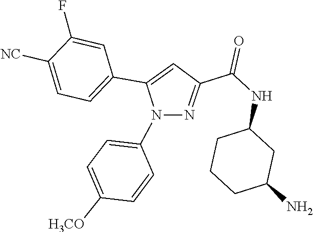 [SMILES: COc1ccc(-n2nc(C(=O)N[C@@H]3CCC[C@H](N)C3)cc2-c2ccc(C#N)c(F)c2)cc1] | N-((1R,3S)-3-aminocyclohexyl)-5-(4-cyano- 3-fluorophenyl)-1-(4-methoxyphenyl)-1H- pyrazole-3-carboxamide; |
| 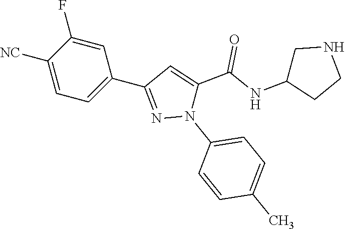 [SMILES: Cc1ccc(-n2nc(-c3ccc(C#N)c(F)c3)cc2C(=O)NC2CCNC2)cc1] | 3-(4-cyano-3-fluorophenyl)-N-(pyrrolidin-3- yl)-1-(p-tolyl)-1H-pyrazole-5-carboxamide; |
| 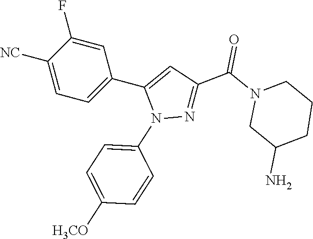 [SMILES: COc1ccc(-n2nc(C(=O)N3CCCC(N)C3)cc2-c2ccc(C#N)c(F)c2)cc1] | 4-(3-(3-Aminopiperidine-1-carbonyl)-1-(4- methoxyphenyl)-1H-pyrazol-5-yl)-2- fluorobenzonitrile; |
| 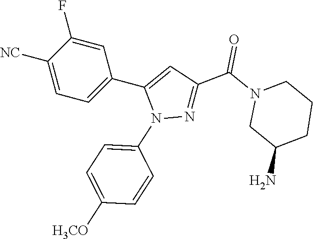 [SMILES: COc1ccc(-n2nc(C(=O)N3CCC[C@@H](N)C3)cc2-c2ccc(C#N)c(F)c2)cc1] | (R)-4-(3-(3-aminopiperidine-1-carbonyl)-1- (4-methoxyphenyl)-1H-pyrazol-5-yl)-2- fluorobenzonitrile; |
| 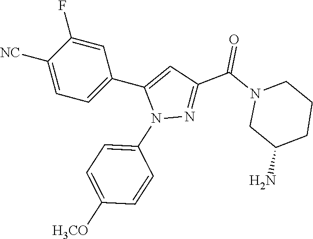 [SMILES: COc1ccc(-n2nc(C(=O)N3CCC[C@H](N)C3)cc2-c2ccc(C#N)c(F)c2)cc1] | (S)-4-(3-(3-aminopiperidine-1-carbonyl)-1-(4- methoxyphenyl)-1H-pyrazol-5-yl)-2- fluorobenzonitrile; |
| 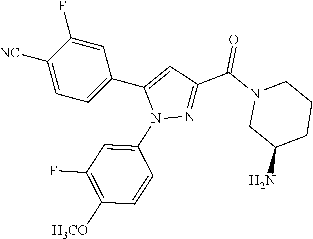 [SMILES: COc1ccc(-n2nc(C(=O)N3CCC[C@@H](N)C3)cc2-c2ccc(C#N)c(F)c2)cc1F] | (R)-4-(3-(3-aminopiperidine-1-carbonyl)-1- (3-fluoro-4-methoxyphenyl)-1H-pyrazol-5- yl)-2-fluorobenzonitrile; |
| 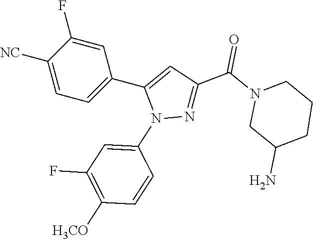 [SMILES: COc1ccc(-n2nc(C(=O)N3CCCC(N)C3)cc2-c2ccc(C#N)c(F)c2)cc1F] | 4-(3-(3-aminopiperidine-1-carbonyl)-1-(3- fluoro-4-methoxyphenyl)-1H-pyrazol-5-yl)-2- fluorobenzonitrile; |
| 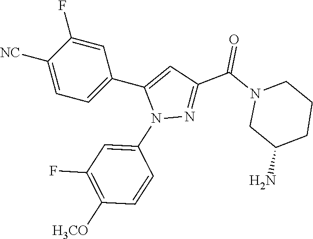 [SMILES: COc1ccc(-n2nc(C(=O)N3CCC[C@H](N)C3)cc2-c2ccc(C#N)c(F)c2)cc1F] | (S)-4-(3-(3-aminopiperidine-1-carbonyl)-1-(3- fluoro-4-methoxyphenyl)-1H-pyrazol-5-yl)-2- fluorobenzonitrile; |
| 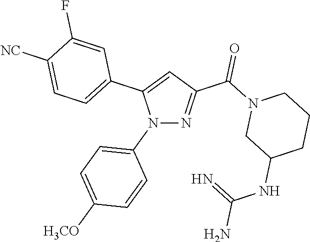 [SMILES: COc1ccc(-n2nc(C(=O)N3CCCC(NC(=N)N)C3)cc2-c2ccc(C#N)c(F)c2)cc1] | 1-(1-(5-(4-cyano-3-fluorophenyl)-1-(4- methoxyphenyl)-1H-pyrazole-3- carbonyl)piperidin-3-yl)guanidine; |
| 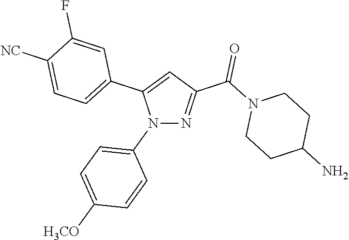 [SMILES: COc1ccc(-n2nc(C(=O)N3CCC(N)CC3)cc2-c2ccc(C#N)c(F)c2)cc1] | 4-(3-(4-aminopiperidine-1-carbonyl)-1-(4- methoxyphenyl)-1H-pyrazol-5-yl)-2- fluorobenzonitrile; |
| 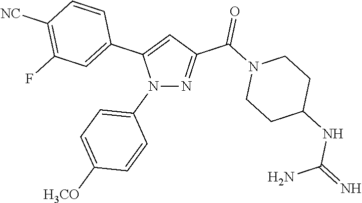 [SMILES: COc1ccc(-n2nc(C(=O)N3CCC(NC(=N)N)CC3)cc2-c2ccc(C#N)c(F)c2)cc1] | 1-(1-(5-(4-cyano-3-fluorophenyl)-1-(4- methoxyphenyl)-1H-pyrazole-3- carbonyl)piperidin-4-yl)guanidine; |
| 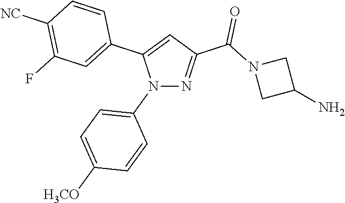 [SMILES: COc1ccc(-n2nc(C(=O)N3CC(N)C3)cc2-c2ccc(C#N)c(F)c2)cc1] | 4-(3-(3-aminoazetidine-1-carbonyl)-1-(4- methoxyphenyl)-1H-pyrazol-5-yl)-2- fluorobenzonitrile; |
| 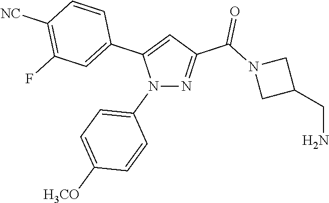 [SMILES: COc1ccc(-n2nc(C(=O)N3CC(CN)C3)cc2-c2ccc(C#N)c(F)c2)cc1] | 4-(3-(3-(aminomethyl)azetidine-1-carbonyl)- 1-(4-methoxyphenyl)-1H-pyrazol-5-yl)-2- fluorobenzonitrile; |
| 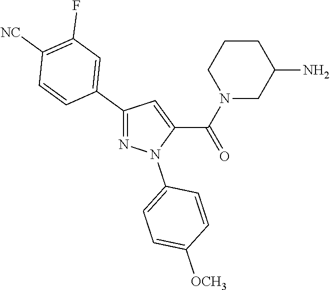 [SMILES: COc1ccc(-n2nc(-c3ccc(C#N)c(F)c3)cc2C(=O)N2CCCC(N)C2)cc1] | 4-(5-(3-aminopiperidine-1-carbonyl)-1-(4- methoxyphenyl)-1H-pyrazol-3-yl)-2- fluorobenzonitrile; |
| 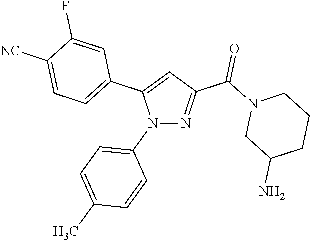 [SMILES: Cc1ccc(-n2nc(C(=O)N3CCCC(N)C3)cc2-c2ccc(C#N)c(F)c2)cc1] | 4-(4-(3-aminopiperidine-1-carbonyl)-1-(p- tolyl)-1H-pyrazol-5-yl)-2-fluorobenzonitrile; |
| 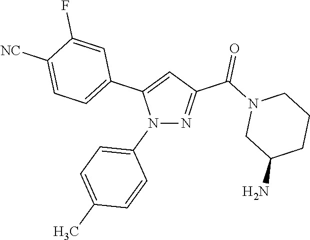 [SMILES: Cc1ccc(-n2nc(C(=O)N3CCC[C@@H](N)C3)cc2-c2ccc(C#N)c(F)c2)cc1] | (R)-4-(3-(3-aminopiperidine-1-carbonyl)-1- (p-tolyl)-1H-pyrazol-5-yl)-2- fluorobenzonitrile; |
| 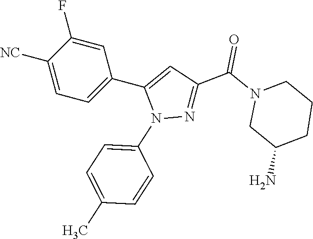 [SMILES: Cc1ccc(-n2nc(C(=O)N3CCC[C@H](N)C3)cc2-c2ccc(C#N)c(F)c2)cc1] | (S)-4-(3-(3-aminopiperidine-1-carbonyl)-1-(p- tolyl)-1H-pyrazol-5-yl)-2-fluorobenzonitrile; |
| 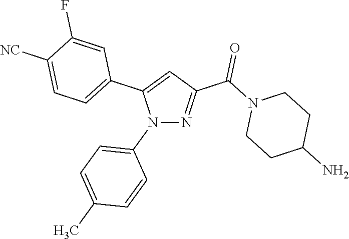 [SMILES: Cc1ccc(-n2nc(C(=O)N3CCC(N)CC3)cc2-c2ccc(C#N)c(F)c2)cc1] | 4-(3-(4-aminopiperidine-1-carbonyl)-1-(p- tolyl)-1H-pyrazol-5-yl)-2-fluorobenzonitrile; |
| 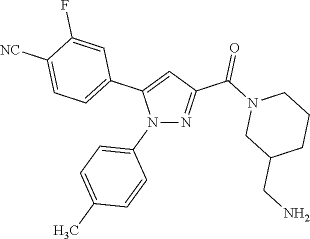 [SMILES: Cc1ccc(-n2nc(C(=O)N3CCCC(CN)C3)cc2-c2ccc(C#N)c(F)c2)cc1] | 4-(3-(3-(aminomethyl)piperidine-1-carbonyl)- 1-(p-tolyl)-1H-pyrazol-5-yl)-2- fluorobenzonitrile; |
| 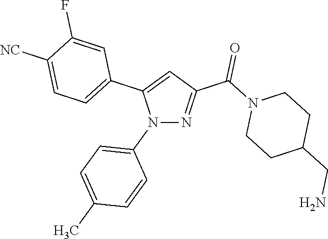 [SMILES: Cc1ccc(-n2nc(C(=O)N3CCC(CN)CC3)cc2-c2ccc(C#N)c(F)c2)cc1] | 4-(3-(4-(aminomethyl)piperidine-1-carbonyl)- 1-(3-fluoro-4-methylphenyl)-1H-pyrazol-5- yl)-2-fluorobenzonitrile; |
| 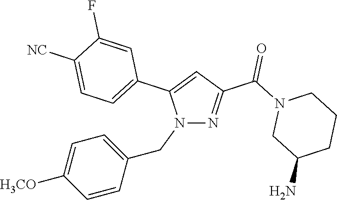 [SMILES: COc1ccc(Cn2nc(C(=O)N3CCC[C@@H](N)C3)cc2-c2ccc(C#N)c(F)c2)cc1] | (R)-4-(3-(3-aminopiperidine-1-carbonyl)-1- (4-methoxybenzyl)-1H-pyrazol-5-yl)-2- fluorobenzonitrile; |
| 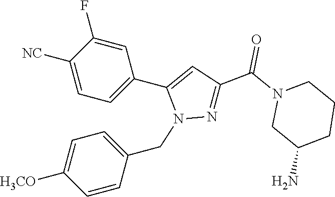 [SMILES: COc1ccc(Cn2nc(C(=O)N3CCC[C@H](N)C3)cc2-c2ccc(C#N)c(F)c2)cc1] | (S)-4-(3-(3-aminopiperidine-1-carbonyl)-1-(4- methoxybenzyl)-1H-pyrazol-5-yl)-2- fluorobenzonitrile; |
| 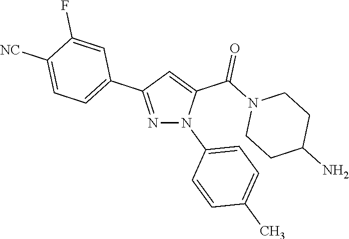 [SMILES: Cc1ccc(-n2nc(-c3ccc(C#N)c(F)c3)cc2C(=O)N2CCC(N)CC2)cc1] | 4-(5-(4-aminopiperidine-1-carbonyl)-1-(p- tolyl)-1H-pyrazol-3-yl)-2-fluorobenzonitrile; |

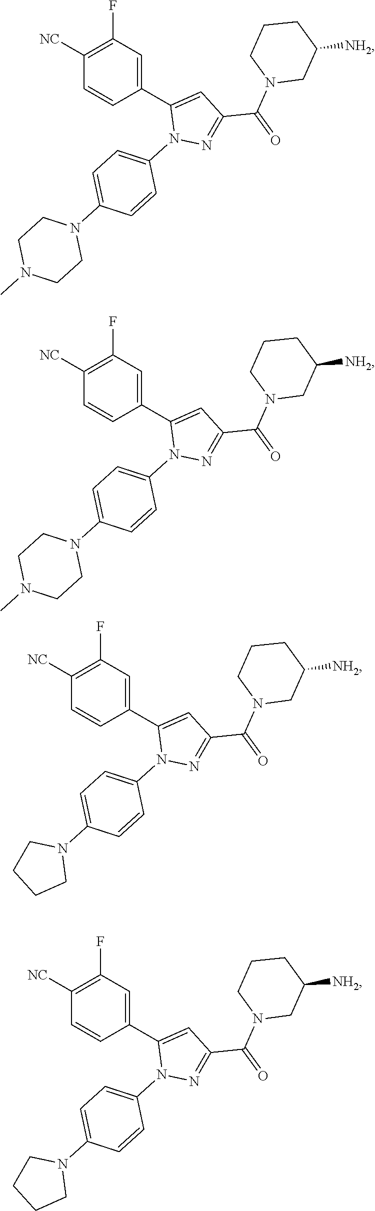 [SMILES: CN1CCN(c2ccc(-n3nc(C(=O)N4CCC[C@H](N)C4)cc3-c3ccc(C#N)c(F)c3)cc2)CC1 ; CN1CCN(c2ccc(-n3nc(C(=O)N4CCC[C@@H](N)C4)cc3-c3ccc(C#N)c(F)c3)cc2)CC1 ; N#Cc1ccc(-c2cc(C(=O)N3CCC[C@H](N)C3)nn2-c2ccc(N3CCCC3)cc2)cc1F ; N#Cc1ccc(-c2cc(C(=O)N3CCC[C@@H](N)C3)nn2-c2ccc(N3CCCC3)cc2)cc1F]

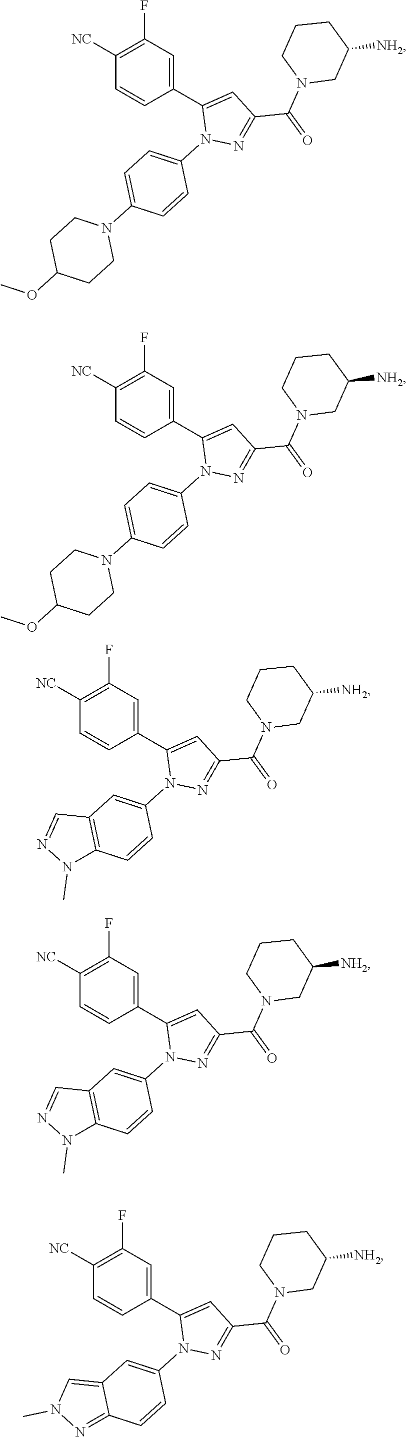

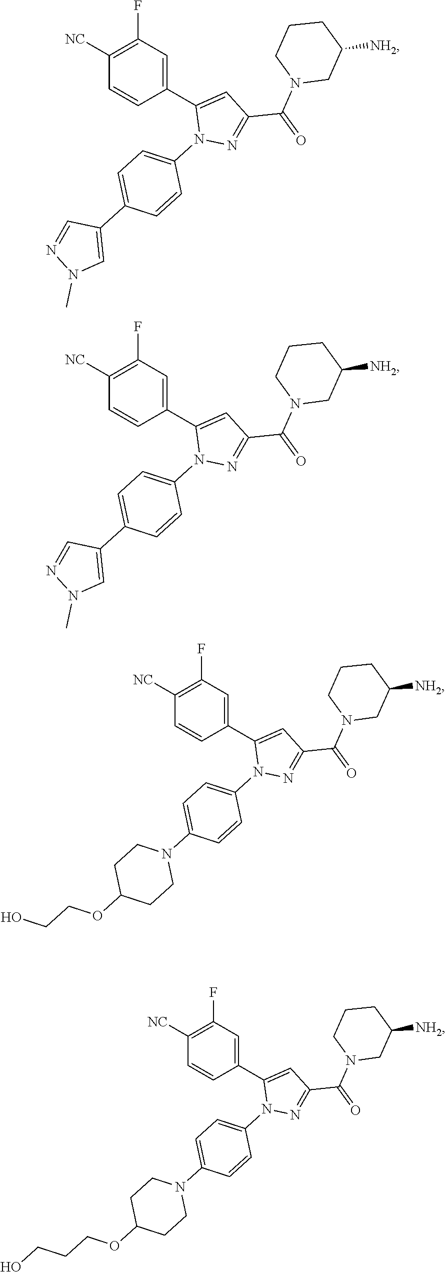 [SMILES: Cn1cc(-c2ccc(-n3nc(C(=O)N4CCC[C@H](N)C4)cc3-c3ccc(C#N)c(F)c3)cc2)cn1 ; Cn1cc(-c2ccc(-n3nc(C(=O)N4CCC[C@@H](N)C4)cc3-c3ccc(C#N)c(F)c3)cc2)cn1 ; N#Cc1ccc(-c2cc(C(=O)N3CCC[C@@H](N)C3)nn2-c2ccc(N3CCC(OCCO)CC3)cc2)cc1F ; N#Cc1ccc(-c2cc(C(=O)N3CCC[C@@H](N)C3)nn2-c2ccc(N3CCC(OCCCO)CC3)cc2)cc1F]

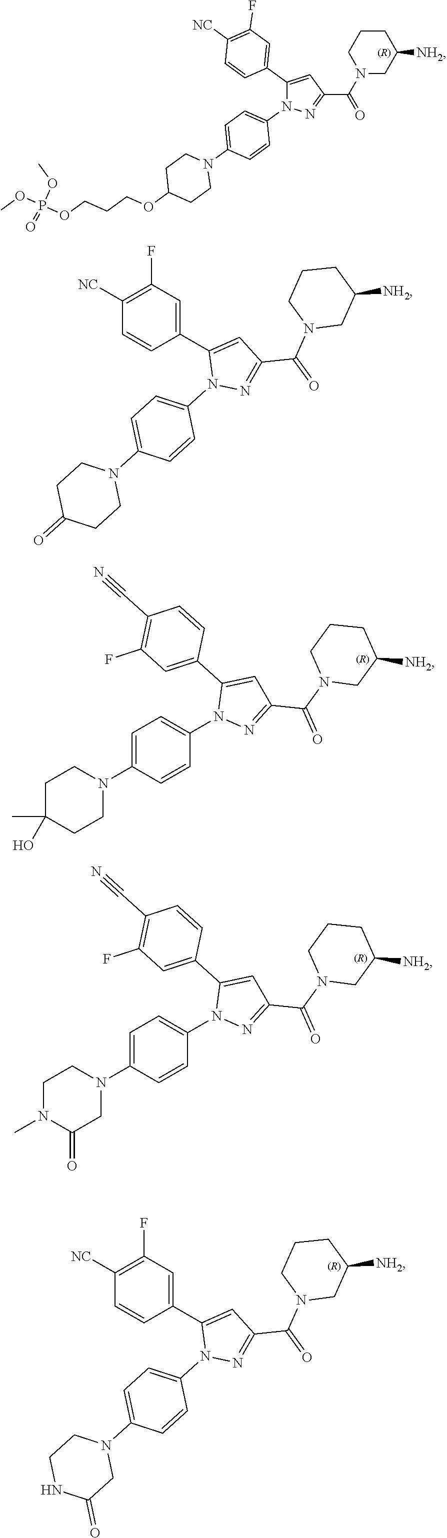

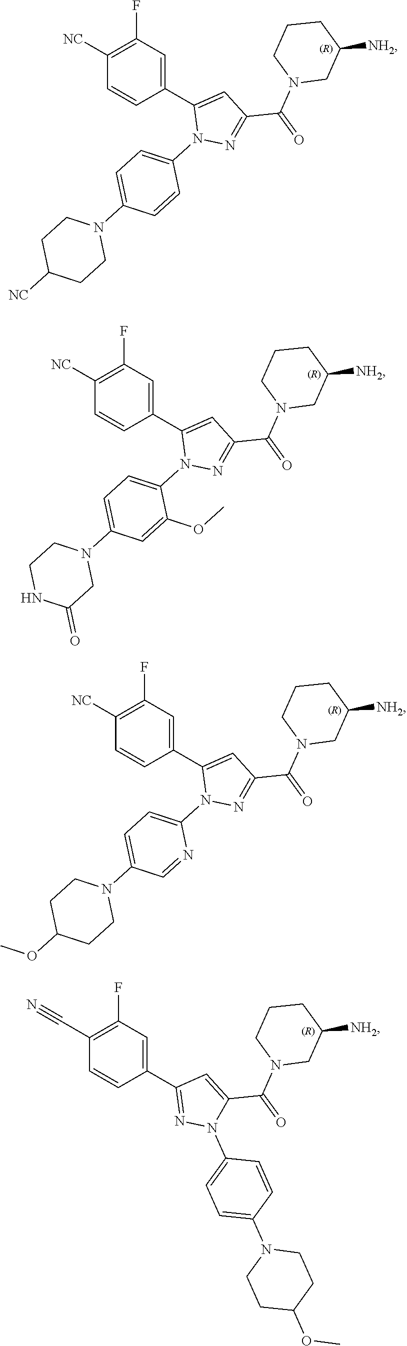

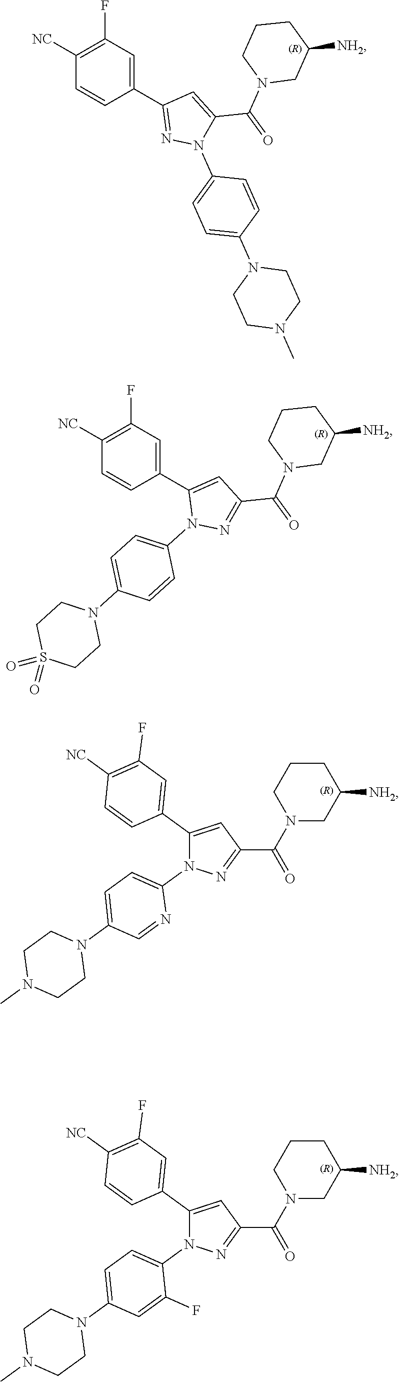

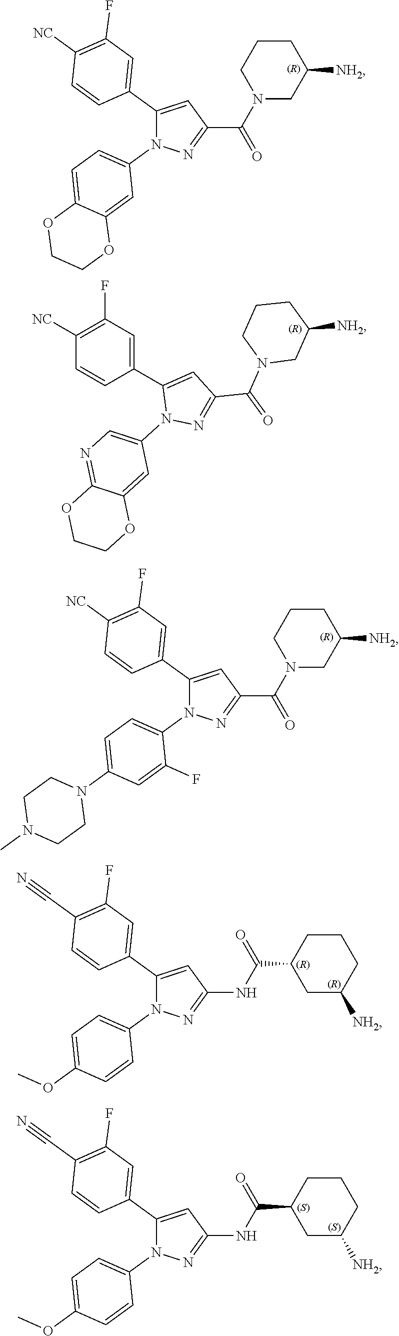

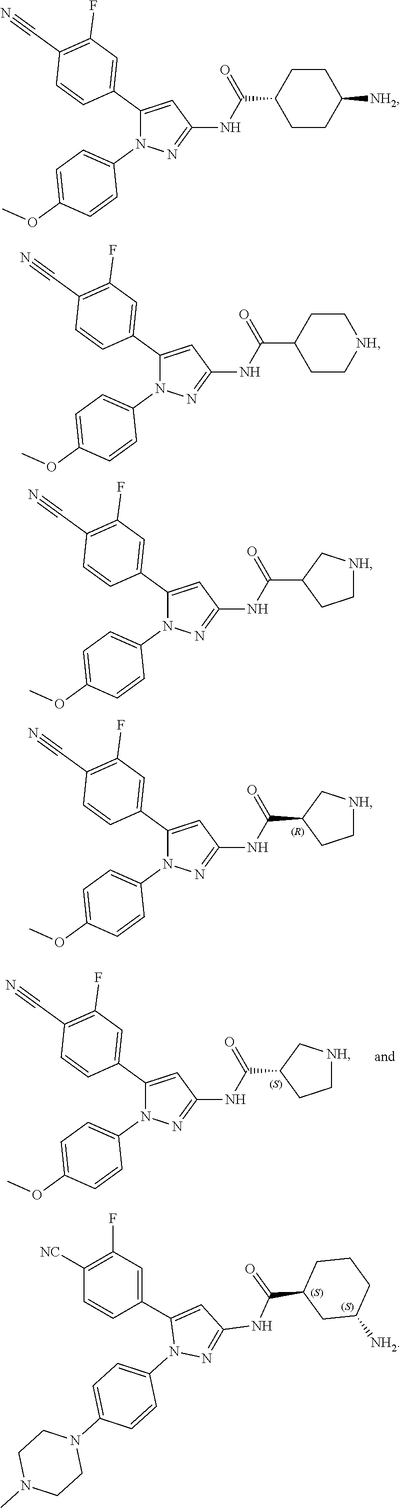

Also provided herein is a compound of Formula I or II:

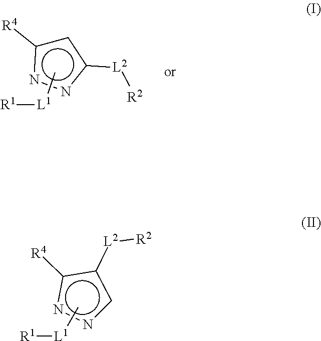

or a salt or tautomer thereof, wherein:
L¹ is a bond;
L² is chosen from —C(O)NH—, —NHC(O)—, and —C(O)—;
R¹ is phenyl substituted with R^{5a};
R² is chosen from cyclohexyl, azetidinyl, pyrrolidinyl, and piperidinyl, any of which is optionally substituted with one or more R⁶;
R⁴ is phenyl which is substituted with one R^{7a} and optionally substituted with one or more R^{7b};
R^{5a} is OCH₃;
each R⁶ is independently chosen from —NHR⁸, —NHC(NH)NHR⁸, and —CH₂NHR⁸;
R^{7a} is cyano;
each R^{7b} is independently chosen from halo; and
each R⁸ is independently chosen from H and C₁₋₃ alkyl.

In some embodiments, L² is —C(O)—.

In some embodiments, L² is —C(O)NH— or —NHC(O)—.

In some embodiments, R¹ is 4-methoxyphenyl.

In some embodiments, R² is chosen from piperidin-1-yl and piperidin-3-yl, either of which is optionally substituted with one or more R⁶.

In some embodiments, R² is piperidin-1-yl, optionally substituted with R⁶.

In some embodiments, R² is 3-aminopiperidin-1-yl.

In some embodiments, R² is piperidin-3-yl, optionally substituted with one or more R⁶.

In some embodiments, R⁶ is chosen from —NHR⁸ and —CH₂NHR⁸.

In some embodiments, R² is piperidin-3-yl.

In some embodiments, R⁴ is phenyl substituted with one R^{7a}.

In some embodiments, R⁴ is 4-cyanophenyl.

In some embodiments, the compound of Formula I is chosen from:

| | |
|---|---|
| 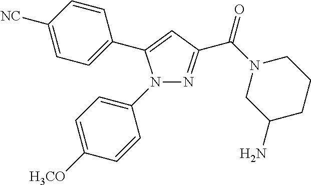 [SMILES: COc1ccc(-n2nc(C(=O)N3CCCC(N)C3)cc2-c2ccc(C#N)cc2)cc1] | 4-(3-(3-Aminopiperidine-1-carbonyl)-1-(4- methoxyphenyl)-1H-pyrazol-5- yl)benzonitrile; |
| 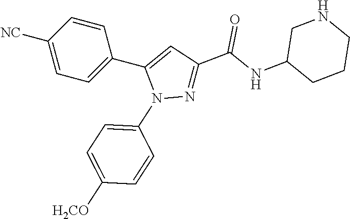 | 5-(4-cyanophenyl)-1-(4-methoxyphenyl)-N- (piperidin-3-yl)-1H-pyrazole-3-carboxamide; |
| 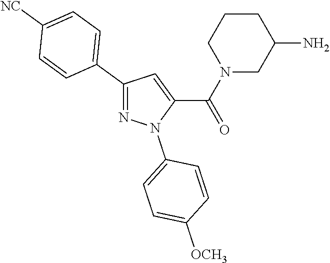 [SMILES: COc1ccc(-n2nc(-c3ccc(C#N)cc3)cc2C(=O)N2CCCC(N)C2)cc1] | 4-(5-(3-aminopiperidine-1-carbonyl)-1-(4- methoxyphenyl)-1H-pyrazol-3- yl)benzonitrile; |
| 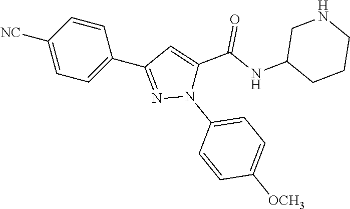 [SMILES: COc1ccc(-n2nc(-c3ccc(C#N)cc3)cc2C(=O)NC2CCCNC2)cc1] | 3-(4-cyanophenyl)-1-(4-methoxyphenyl)-N- (piperidin-3-yl)-1H-pyrazole-5-carboxamide; |
| 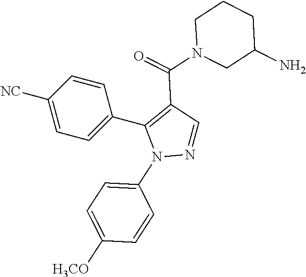 [SMILES: COc1ccc(-n2ncc(C(=O)N3CCCC(N)C3)c2-c2ccc(C#N)cc2)cc1] | 4-(4-(3-aminopiperidine-1-carbonyl)-1-(4- methoxyphenyl)-1H-pyrazol-5- yl)benzonitrile; |
| 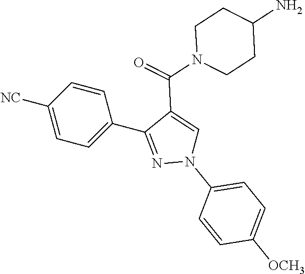 [SMILES: COc1ccc(-n2cc(C(=O)N3CCC(N)CC3)c(-c3ccc(C#N)cc3)n2)cc1] | 4-(4-(4-aminopiperidine-1-carbonyl)-1-(4- methoxyphenyl)-1H-pyrazol-3- yl)benzonitrile; and |
| 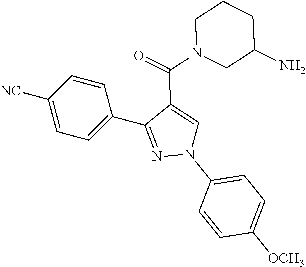 [SMILES: COc1ccc(-n2cc(C(=O)N3CCCC(N)C3)c(-c3ccc(C#N)cc3)n2)cc1] | 4-(4-(3-aminopiperidine-1-carbonyl)-1-(4- methoxyphenyl)-1H-pyrazol-3- yl)benzonitrile. |

Also provided herein is a pharmaceutical composition comprising a compound as recited herein, or a salt or tautomer thereof, together with a pharmaceutically acceptable carrier.

In some embodiments, the pharmaceutical composition is formulated for oral administration.

In some embodiments, the pharmaceutical composition additionally comprises another therapeutic agent.

Also provided herein is a method of inhibition of KDM1A comprising contacting KDM1A with a compound as recited herein, or a salt or tautomer thereof, or a pharmaceutical composition as recited herein.

Also provided herein is a method of treatment of a KDM1A-mediated disease comprising the administration of a therapeutically effective amount of a compound as recited herein, or a salt or tautomer thereof, or a pharmaceutical composition as recited herein, to a patient in need thereof.

In some embodiments, the disease is cancer.

In some embodiments, the cancer is chosen from Ewing's sarcoma, multiple myeloma, T-cell leukemia, Wilm's tumor, small-cell lung cancer, bladder cancer, prostate cancer, breast cancer, head/neck cancer, colon cancer, and ovarian cancer.

In some embodiments, the disease is a myeloid disease.

In some embodiments, the myeloid disease is chosen from Myelofibrosis, Polycythemia Vera, Essential Thrombocythemia, Myelodysplastic Syndrome (MDS), Acute Myelogenous Leukemia (AML), Chronic Neutrophilic Leukemia (CNL), Chronic Eosinophilic Leukemia (CEL), Hypereosinophilic Syndrome, Chronic Myelogenous Leukemia (CVL), and Atypical CML.

In some embodiments, the disease is an inflammatory disease.

In some embodiments, the inflammatory disease is chosen from inflammatory bowel disease, rheumatoid arthritis, or systemic lupus erythematosus.

Also provided herein is a method of treatment of a disease resulting from mutations in globin genes which affect expression or function of the globin protein, comprising the administration of a therapeutically effective amount of a compound as recited herein, or a salt or tautomer thereof, or a pharmaceutical composition as recited herein, to a patient in need thereof.

Also provided herein is a method for achieving an effect in a patient comprising the administration of a therapeutically effective amount of a compound as recited herein, or a salt or tautomer thereof, or a pharmaceutical composition as recited herein, to a patient, wherein the effect is chosen from an elevation of red blood cell count, an elevation of the red blood cell count of red cells containing fetal hemoglobin, an elevation in the total concentration of fetal hemoglobin in red cells, an elevation in the total concentration of fetal hemoglobin in reticulocytes, an increase in the transcription of the gamma globin gene in bone marrow-derived red cell precursors, e.g., pro-erythroblasts, a reduction in the number of sickle cell crises a patient experiences over a unit period of time, a halt to or prevention of tissue damage e.g., in the heart, spleen, brain or kidney caused by sickling cells, a reduction in the proportion of red cells that undergo sickling under physiological conditions of relative hypoxia as measured using patient blood in an in vitro assay, an increase in the amount of histone 3 lysine methylation at lysine position 4 (H3K4me1 and H3K4me2), and/or a decrease in the amount of histone 3 methylation at lysine position 9 (H3K9me1 or H3K4me2) near or at the gamma globin promoter as assayed by ChIP using cells derived from a treated patient.

Also provided herein is a method of inhibiting at least one KDM1A function comprising the step of contacting KDM1A with a compound as recited herein, or a salt or tautomer thereof, or a pharmaceutical composition as recited herein, wherein the inhibition is measured by phenotype of red cells or their precursors either cultured or in vivo in humans or mouse or transgenic mice containing the human beta globin locus or portions thereof, the ability of cancer cells to proliferate, become differentiated, or induced to undergo apoptosis, the expression of specific genes known to be regulated by KDM1A activity such as gamma globin or HOXA9, a change in the histone methylation states, a change in the methylation state of proteins known to be demethylated by KDM1A such as G9a, p53, DNMT1 or SUV39H1, expression of KDM1A-regulated genes, or binding of KDM1A with a natural binding partner such as CoREST, NuRD, DNMT1 or HDACs.

Also provided herein is a method of treatment of a bone marrow failure syndrome, comprising the administration of a therapeutically effective amount of a compound as recited herein, or a salt or tautomer thereof, or a pharmaceutical composition as recited herein, to a patient in need thereof.

In some embodiments, the bone marrow failure syndrome is an inherited bone marrow failure syndrome.

In some embodiments, the inherited bone marrow failure syndrome is chosen from Fanconi anemia, dyskeratosis congenita and other telomere biology disorders, Shwachman-Diamond syndrome, Diamond-Blackfan anemia, aplastic anemia, reticular dysgenesis, a GATA2-related disorder, a SAMD9 SAMD9L-related disorder, congenital amegakaryocytic thrombocytopenia, ADA2 deficiency, MIRAGE (major findings of Myelodysplasia, Infection, Restriction of growth, Adrenal hypoplasia, Genital phenotypes, and Enteropathy) syndrome, Paroxysmal nocturnal hemoglobinuria (PNH), Pearson syndrome, thrombocytopenia with absent radii (TAR), radiation or chemical poisoning of the bone marrow including chemotherapy, and a congenital neutropenia (e.g., one associated with a gene chosen from ELANE, HAX1, G6PC3, GFIl, CSF3R, X-linked WAS, CXCR4, VPS45A, and JAGNI).

Also provided herein is a method of treatment of a hemoglobinopathy, comprising the administration of a therapeutically effective amount of a compound as recited herein, or a salt or tautomer thereof, or a pharmaceutical composition as recited herein, to a patient in need thereof.

In some embodiments, the hemoglobinopathy is chosen from thalassemia major, sickle cell disease, hemoglobin E/thalassemia, and thalassemia intermedia.

In some embodiments, the method of treatment further comprises administration of another therapeutic agent.

In some embodiments, the other therapeutic agent is a DNA methyltransferase inhibitor.

In some embodiments, the DNA methyltransferase inhibitor is chosen from decitabine and 5′-aza-cytadine.

### Abbreviations and Definitions

To facilitate understanding of the disclosure, a number of terms and abbreviations as used herein are defined below as follows:

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e., A alone, B alone or A and B in combination. The expression “A, B and/or C” is intended to mean A alone, B alone, C alone, A and B in combination, A and C in combination, B and C in combination or A, B, and C in combination.

The term “about,” as used herein when referring to a measurable value such as an amount of a compound, dose, time, temperature, and the like, is meant to encompass variations of 20%, 10%, 5%, 1%, 0.5%, or even 0.1% from the specified amount.

A “therapeutically effective amount” of a drug is an amount of drug or its pharmaceutically acceptable salt that eliminates, alleviates, or provides relief of the symptoms of the disease for which it is administered.

A “subject in need thereof” is a human or non-human animal that exhibits one or more symptoms or indicia of a disease.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.). When n is set at 0 in the context of “0 carbon atoms”, it is intended to indicate a bond or null.

The term “alkoxy,” as used herein, alone or in combination, refers to an alkyl ether group, wherein the term alkyl is as defined below. Examples of suitable alkyl ether groups include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl group containing from 1 to 20 carbon atoms. In certain embodiments, said alkyl will comprise from 1 to 10 carbon atoms. In further embodiments, said alkyl will comprise from 1 to 6 carbon atoms. Examples of alkyl groups include methyl, ethyl, n-propyl, isopropyl, n-butyl, isobutyl, sec-butyl, tert-butyl, pentyl, iso-amyl, hexyl, octyl, noyl, and the like.

The term “alkylene,” as used herein, alone or in combination, refers to a saturated aliphatic group derived from a straight or branched chain saturated hydrocarbon attached at two or more positions, such as methylene (—CH₂—).

The term “aryl”, as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The terms “benzo” and “benz,” as used herein, alone or in combination, refer to the divalent group C₆H₄=derived from benzene. Examples include benzothiophene and benzimidazole.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic, or tricyclic alkyl group, wherein each cyclic moiety contains from 3 to 12 carbon atom ring members, and which may optionally be a benzo fused ring system. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ether,” as used herein, alone or in combination, refers to an oxy group bridging two moieties linked at carbon atoms.

The term “guanidine”, as used herein, alone or in combination, refers to —NHC(═NH)NH₂, or the corresponding guanidinium cation.

The term “halo,” or “halogen,” as used herein, alone or in combination, refers to fluorine, chlorine, bromine, or iodine.

The term “heteroaryl,” as used herein, alone or in combination, refers to a 3- to 7-membered unsaturated heteromonocyclic ring, or a fused monocyclic, bicyclic, or tricyclic ring system in which at least one of the fused rings is aromatic, which contains at least one atom chosen from O, S, and N. In certain embodiments, said heteroaryl will comprise from 5 to 7 carbon atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings.

The terms “heterocycloalkyl” and, interchangeably, “heterocycle,” as used herein, alone or in combination, each refer to a saturated or partially unsaturated monocyclic, bicyclic, or tricyclic heterocyclic group containing at least one heteroatom as a ring member, wherein each said heteroatom may be independently chosen from nitrogen, oxygen, and sulfur. In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an additional heterocycle group.

The term “hydroxy,” as used herein, alone or in combination, refers to —OH.

The terms “oxy” or “oxa,” as used herein, alone or in combination, refer to —O—.

The term “oxo,” as used herein, alone or in combination, refers to ═O.

The term “phosphonate,” as used herein, alone or in combination, refers to a —P(═O)(OR)₂ group, wherein R is chosen from alkyl and aryl. The term “phosphonic acid”, as used herein, alone or in combination, refers to a —P(═O)(OH)₂ group.

Any definition herein may be used in combination with any other definition to describe a composite structural group. By convention, the trailing element of any such definition is that which attaches to the parent moiety. For example, the composite group alkylamino would represent an alkyl group attached to the parent molecule through an amino group, and the term alkoxyalkyl would represent an alkoxy group attached to the parent molecule through an alkyl group.

Asymmetric centers exist in the compounds disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present disclosure includes all cis, trans, syn, anti, entgegen (E), and zusammen (Z) isomers as well as the appropriate mixtures thereof. Additionally, compounds may exist as tautomers; all tautomeric isomers are provided by this disclosure. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The phrase “therapeutically effective” is intended to qualify the amount of active ingredients used in the treatment of a disease or disorder. This amount will achieve the goal of reducing or eliminating the said disease or disorder.

The term “therapeutically acceptable” refers to those compounds or salts thereof which are suitable for use in contact with the tissues of patients without undue toxicity, irritation, and allergic response, are commensurate with a reasonable benefit/risk ratio, and are effective for their intended use.

As used herein the term “treat,” “treating,” or “treatment” refers to the administration of therapy to an individual (i.e., a human) who already manifests at least one symptom of a disease or condition or who has previously manifested at least one symptom of a disease or condition. For example, “treating” can include alleviating, abating, or ameliorating a disease or condition symptoms, preventing additional symptoms, ameliorating the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with a particular disorder. Therefore, treating a disorder does not necessarily mean a reduction in severity of all symptoms associated with a disorder and does not necessarily mean a complete reduction in the severity of one or more symptoms associated with a disorder.

The compounds disclosed herein can exist as therapeutically acceptable salts. The present disclosure includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like.

Basic addition salts can be prepared during the final isolation and purification of the compounds by reaction of a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, and piperazine.

A salt of a compound can be made by reaction of the appropriate compound, in the form of the free base, with the appropriate acid.

While it may be possible for the compounds disclosed herein to be administered as the raw chemical, it is also possible to present them as pharmaceutical formulations (equivalently, “pharmaceutical compositions”). Accordingly, provided herein are pharmaceutical formulations which comprise one or more of certain compounds disclosed herein, or one or more pharmaceutically acceptable salts thereof, together with one or more pharmaceutically acceptable carriers thereof and optionally one or more other therapeutic ingredients. The carrier(s) must be “acceptable” in the sense of being compatible with the other ingredients of the formulation and not rious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping, or compression processes.

Compounds may be administered at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

Preferred unit dosage formulations are those containing an effective dose, as herein below recited, or an appropriate fraction thereof, of the active ingredient. In certain embodiments, a formulation disclosed herein is administered once a day. However, the formulations may also be formulated for administration at any frequency of administration, including once a week, once every 5 days, once every 3 days, once every 2 days, twice a day, three times a day, four times a day, five times a day, six times a day, eight times a day, every hour, or any greater frequency. Such dosing frequency is also maintained for a varying duration of time depending on the therapeutic regimen. The duration of a particular therapeutic regimen may vary from one-time dosing to a regimen that extends for months or years. The formulations are administered at varying dosages, but typical dosages are one to two drops at each administration, or a comparable amount of a gel or other formulation. One of ordinary skill in the art would be familiar with determining a therapeutic regimen for a specific indication.

The amount of active ingredient that may be combined with the carrier materials to produce a single dosage form will vary depending upon the host treated and the particular mode of administration. Similarly, the precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. In addition, the route of administration may vary depending on the condition and its severity.

In certain instances, it may be appropriate to administer at least one of the compounds described herein (or a pharmaceutically acceptable salt thereof) in combination with another therapeutic agent. By way of example only, if one of the side effects experienced by a patient upon receiving one of the compounds herein is inflammation, then it may be appropriate to administer an anti-inflammatory agent in combination with the initial therapeutic agent. Alternatively, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). There is even the possibility that two compounds, one of the compounds described herein and a second compound may together achieve the desired therapeutic effect that neither alone could achieve. Alternatively, by way of example only, the benefit experienced by a patient may be increased by administering one of the compounds described herein with another therapeutic agent (which also includes a therapeutic regimen) that also has therapeutic benefit. By way of example only, in a treatment for acute myelogenous leukemia or sickle cell anemia involving administration of one of the compounds described herein, increased therapeutic benefit may result by also providing the patient with another therapeutic agent for sickle cell anemia or for acute myelogenous leukemia. In any case, regardless of the disease, disorder, or condition being treated, the overall benefit experienced by the patient may simply be additive of the two therapeutic agents or the two agents may have synergistic therapeutic effects in a patient.

Effective combination therapy may be achieved with a single composition or pharmacological formulation that includes both agents, or with two distinct compositions or formulations, at the same time, wherein one composition includes a compound of the present disclosure, and the other includes the second agent(s). Alternatively, the therapy may precede or follow the other agent treatment by intervals ranging from minutes to months. Administration of the compounds of the present disclosure to a patient will follow general protocols for the administration of pharmaceuticals, taking into account the toxicity, if any, of the drug. It is expected that the treatment cycles would be repeated as necessary.

Specific, non-limiting examples of possible combination therapies include use of certain compounds of the disclosure with the following agents and classes of agents: agents that inhibit DNA methyltransferases such as decitabine or 5′-aza-cytadine; agents that inhibit the activity of histone deacetylases, histone de-sumoylases, histone de-ubiquitinases, or histone phosphatases such as hydroxyurea; antisense RNAs that might inhibit the expression of other components of the protein complex bound at the DR site in the gamma globin promoter; agents that inhibit the action of Klf1 or the expression of KLF1; agents that inhibit the action of BCL11a or the expression of BCL11A; and agents that inhibit cell cycle progression such as hydroxyurea, ara-C, or daunorubicin; agents that induce differentiation in leukemic cells such as all-trans retinoic acid (ATRA).

Thus, in another aspect, the present disclosure provides methods for treating diseases or disorders in a human or animal subject in need of such treatment comprising administering to said subject an amount of a compound disclosed herein effective to reduce or prevent said disorder in the subject, optionally in combination with at least one additional agent for the treatment of said disorder that is known in the art.

Used either as a monotherapy or in combination with other agents, the compounds disclosed herein are useful in the prevention and/or treatment of beta-hemoglobinopathies such as thalassemia major, sickle cell disease, hemoglobin E/thalassemia, and thalassemia intermedia.

The compounds disclosed herein can be used in the treatment of diseases in which an increase in transcription through the manipulation of epigenetic regulatory factors such as inhibition of KDM1A would be beneficial to the patient. This applies to diseases including but not limited to loss of function mutations, mutations resulting in haploinsufficiency, deletions and duplications of genetic material or epigenetic regulatory mechanisms have altered the normal expression pattern of a gene or genes that has the effect of altering the dose of a gene product(s). Such diseases may include diseases both acquired and hereditary in which the expression of, for example, cytokines affecting immune function, are altered, X-linked mental retardation and other forms of compromised cognitive or motor function such as Alzheimer and Parkinson disease whether they are the acquired or hereditary forms, lipid disorders such as elevated cholesterol, low density lipoprotein, very low density lipoprotein or triglycerides, both type one and type two diabetes, and Mendelian genetic diseases.

Other disorders or conditions that can be advantageously treated by the compounds disclosed herein include inflammation and inflammatory conditions. Inflammatory conditions include, without limitation: arthritis, including sub-types and related conditions such as rheumatoid arthritis, spondyloarthropathies, gouty arthritis, osteoarthritis, systemic lupus erythematosus, juvenile arthritis, acute rheumatic arthritis, enteropathic arthritis, neuropathic arthritis, psoriatic arthritis, and pyogenic arthritis; osteoporosis, tendonitis, bursitis, and other related bone and joint disorders; gastrointestinal conditions such as reflux esophagitis, diarrhea, inflammatory bowel disease, Crohn's disease, gastritis, irritable bowel syndrome, ulcerative colitis, acute and chronic inflammation of the pancreas; pulmonary inflammation, such as that associated with viral infections and cystic fibrosis; skin-related conditions such as psoriasis, eczema, burns, sunburn, dermatitis (such as contact dermatitis, atopic dermatitis, and allergic dermatitis), and hives; pancreatitis, hepatitis, pruritus and vitiligo. In addition, compounds of disclosure are also useful in organ transplant patients either alone or in combination with conventional immunomodulators.

Autoimmune disorders may be ameliorated by the treatment with compounds disclosed herein. Autoimmune disorders include Crohn's disease, ulcerative colitis, dermatitis, dermatomyositis, diabetes mellitus type 1, Goodpasture's syndrome, Graves' disease, Guillain-Barre syndrome (GBS), autoimmune encephalomyelitis, Hashimoto's disease, idiopathic thrombocytopenic purpura, lupus erythematosus, mixed connective tissue disease, multiple sclerosis (MS), myasthenia gravis, narcolepsy, pemphigus vulgaris, pernicious anemia, psoriasis, psoriatic arthritis, polymyositis, primary biliary cirrhosis, rheumatoid arthritis, Sjögren's syndrome, scleroderma, temporal arteritis (also known as “giant cell arteritis”), vasculitis, and Wegener's granulomatosis.

The compounds disclosed herein are also useful for the treatment of organ and tissue injury associated with severe burns, sepsis, trauma, wounds, and hemorrhage- or resuscitation-induced hypotension, and also in such diseases as vascular diseases, migraine headaches, periarteritis nodosa, thyroiditis, aplastic anemia, Hodgkin's disease, scleroderma, rheumatic fever, type I diabetes, neuromuscular junction disease including myasthenia gravis, white matter disease including multiple sclerosis, sarcoidosis, nephritis, nephrotic syndrome, Behcet's syndrome, polymyositis, gingivitis, periodontitis, swelling occurring after injury, ischemias including myocardial ischemia, cardiovascular ischemia, and ischemia secondary to cardiac arrest, and the like.

The compounds disclosed herein are also useful for the treatment of certain diseases and disorders of the nervous system. Central nervous system disorders in KDM1A inhibition is useful include cortical dementias including Alzheimer's disease, central nervous system damage resulting from stroke, ischemias including cerebral ischemia (both focal ischemia, thrombotic stroke and global ischemia (for example, secondary to cardiac arrest), and trauma. Neurodegenerative disorders in which KDM1A inhibition is useful include nerve degeneration or nerve necrosis in disorders such as hypoxia, hypoglycemia, epilepsy, and in cases of central nervous system (CNS) trauma (such as spinal cord and head injury), hyperbaric oxygen-induced convulsions and toxicity, dementia e.g., pre-senile dementia, and AIDS-related dementia, cachexia, Sydenham's chorea, Huntington's disease, Parkinson's Disease, amyotrophic lateral sclerosis (ALS), Korsakoff s disease, cognitive disorders relating to a cerebral vessel disorder, hypersensitivity, sleeping disorders, schizophrenia, depression, depression or other symptoms associated with Premenstrual Syndrome (PMS), and anxiety.

Still other disorders or conditions advantageously treated by the compounds disclosed herein include the prevention or treatment of hyperproliferative diseases, especially cancers, either alone or in combination with standards of care especially those agents that target tumor growth by re-instating tumor suppressor genes in the malignant cells. Hematological and non-hematological malignancies which may be treated or prevented include but are not limited to multiple myeloma, acute and chronic leukemias and hematopoietic proliferative and neoplastic disorders including myelodysplastic syndrome (MDS), acute myelogenous leukemia (AML), Acute Lymphocytic Leukemia (ALL), Chronic Lymphocytic Leukemia (CLL), and chronic myelogenous leukemia (CML), lymphomas, including Hodgkin's lymphoma and non-Hodgkin's lymphoma (low, intermediate, and high grade), as well as solid tumors and malignancies of the brain, head and neck, breast, lung (including non-small-cell lung cancer), reproductive tract, upper digestive tract, pancreas, liver, renal system, bladder, prostate, and colorectal. The present compounds and methods can also be used to treat fibrosis, such as that which occurs with radiation therapy. The present compounds and methods can be used to treat subjects having or prevent the progression of adenomatous polyps, including those with familial adenomatous polyposis (FAP) or sarcoidosis. Non-cancerous proliferative disorders additionally include psoriasis, eczema, and dermatitis.

The present compounds may also be used in co-therapies, partially or completely, in place of other conventional anti-inflammatory therapies, such as together with steroids, NSAIDs, COX-2 selective inhibitors, 5-lipoxygenase inhibitors, LTB₄ antagonists, and LTA₄ hydrolase inhibitors. The compounds disclosed herein may also be used to prevent tissue damage when therapeutically combined with antibacterial or antiviral agents.

The compounds disclosed herein are also useful for the treatment of treat metabolic disorders. KDM1A, using flavin adenosine dinucleotide (FAD) as a cofactor, epigenetically regulates energy-expenditure genes in adipocytes depending on the cellular FAD availability. Additionally, loss of KDM1A function induces a number of regulators of energy expenditure and mitochondrial metabolism resulting in the activation of mitochondrial respiration. Furthermore, in the adipose tissues from mice fed a high-fat diet, expression of KDM1A-target genes is reduced.

Metabolic syndrome (also known as metabolic syndrome X) is characterized by having at least three of the following symptoms: insulin resistance; abdominal fat—in men this is defined as a 40 inch waist or larger, in women 35 inches or larger; high blood sugar levels—at least 110 milligrams per deciliter (mg/dL) after fasting; high triglycerides—at least 150 mg/dL in the blood stream; low HDL—less than 40 mg/dL; pro-thrombotic state (e.g., high fibrinogen or plasminogen activator inhibitor in the blood); or blood pressure of 130/85 mmHg or higher. A connection has been found between metabolic syndrome and other conditions such as obesity, high blood pressure and high levels of LDL cholesterol, all of which are risk factors for cardiovascular diseases. For example, an increased link between metabolic syndrome and atherosclerosis has been shown. People with metabolic syndrome are also more prone to developing type 2 diabetes, as well as PCOS (polycystic ovarian syndrome) in women and prostate cancer in men.

As described above, insulin resistance can be manifested in several ways, including type 2 diabetes. Type 2 diabetes is the condition most obviously linked to insulin resistance. Compensatory hyperinsulinemia helps maintain normal glucose levels often for decades before overt diabetes develops. Eventually the beta cells of the pancreas are unable to overcome insulin resistance through hypersecretion. Glucose levels rise and a diagnosis of diabetes can be made. Patients with type 2 diabetes remain hyperinsulinemic until they are in an advanced stage of disease. As described above, insulin resistance can also correlate with hypertension. One half of patients with essential hypertension are insulin resistant and hyperinsulinemic, and there is evidence that blood pressure is linked to the degree of insulin resistance. Hyperlipidemia, too, is associated with insulin resistance. The lipid profile of patients with type 2 diabetes includes increased serum very-low-density lipoprotein (VLDL) cholesterol and triglyceride levels and, sometimes, a decreased low-density lipoprotein (LDL) cholesterol level. Insulin resistance has been found in persons with low levels of high-density lipoprotein HDL). Insulin levels have also been linked to VLDL synthesis and plasma triglyceride levels.

Specific metabolic diseases and symptoms to be treated by the compounds, compositions, and methods disclosed herein are those mediated at least in part by KDM1A. Accordingly, disclosed herein are methods: for treating insulin resistance in a subject; for reducing glycogen accumulation in a subject; for raising HDL or HDLc, lowering LDL or LDLc, shifting LDL particle size from small dense to normal LDL, lowering VLDL, lowering triglycerides, or inhibiting cholesterol absorption in a subject; for reducing insulin resistance, enhancing glucose utilization or lowering blood pressure in a subject; for reducing visceral fat in a subject; for reducing serum transaminases in a subject; for inducing mitochondrial respiration in a subject; or for treating disease; all comprising the administration of a therapeutic amount of a compound as described herein, to a patient in need thereof. In further embodiments, the disease to be treated may be a metabolic disease. In further embodiments, the metabolic disease may be selected from the group consisting of obesity, diabetes mellitus, especially Type 2 diabetes, hyperinsulinemia, glucose intolerance, metabolic syndrome X, dyslipidemia, hypertriglyceridemia, hypercholesterolemia, and hepatic steatosis. In other embodiments, the disease to be treated may be selected from the group consisting of cardiovascular diseases including vascular disease, atherosclerosis, coronary heart disease, cerebrovascular disease, heart failure, and peripheral vessel disease. In some embodiments, the methods above do not result in the induction or maintenance of a hypoglycemic state.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### General Synthetic Methods for Preparing Compounds

The following schemes can be used to prepare the compounds disclosed herein.

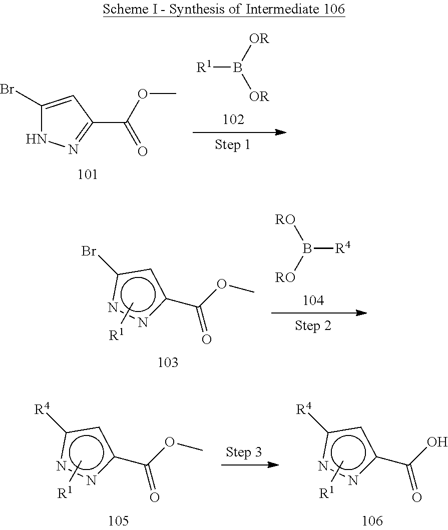

Referring to Scheme I, Step 1, to a solution of the compound of Formula 101 in an organic solvent, such as methylene chloride, is added a compound of Formula 102 (R=alkyl, such as methyl, or hydrogen, or in combination with a second R group forms a heterocycloalkyl, such as pinacol borane), a catalyst, such as copper (II) acetate, and a base, such as triethylamine. The mixture is stirred at ambient temperature for 12-24 h. During this time, the progress of the reaction can be followed by chromatography, for example, LCMS. The product, a compound of Formula 103, is isolated and purified using methods known in the art.

Referring to Scheme I, Step 2, to a solution of the compound of Formula 103 in a polar solvent, such as dioxane, is added a compound of Formula 104, a catalyst, such as Pd(dppf)Cl₂, and a base, such as potassium carbonate, in an inert atmosphere. The mixture is stirred at elevated temperature for between 1-3 h. During this time, the progress of the reaction can be followed by chromatography, for example, LCMS. The product, a compound of Formula 105, is isolated and purified using methods known in the art.

Referring to Scheme I, Step 3, to a solution of the compound of Formula 105 in an organic solvent, such as THF, is added water and a strong base, such as lithium hydroxide. The mixture is stirred at ambient temperature for 1-3 h, then acidified to between pH 4-6 with a strong acid, such as HCl. During this time, the progress of the reaction can be followed by chromatography, for example, LCMS. The product, an intermediate of Formula 106, is isolated and purified using methods known in the art.

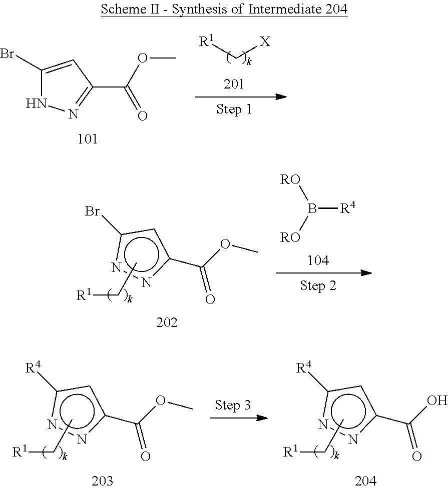

Referring to Scheme II, Step 1, to a solution of the compound of Formula 101 in a polar aprotic solvent, such as dimethylformamide, is added a compound of Formula 201 (X=halogen) and a base, such as potassium carbonate. The mixture is stirred at ambient temperature for 12-24 h. Alternatively, to a compound of Formula 101 in a polar aprotic solvent, such as THF, is added a compound of Formula 201 (X=hydroxyl), an azodicarboxylate, and an organophosphine, such as triphenylphosphine, and the mixture is stirred at ambient temperature for 2-4 h. During this time, the progress of the reaction can be followed by chromatography, for example, LCMS. The product, a compound of Formula 202, is isolated and purified using methods known in the art.

Referring to Scheme II, Steps 2 and 3, the compound of Formula 202 is converted to an intermediate of Formula 204 using similar procedures as those of Scheme I, Steps 2 and 3.

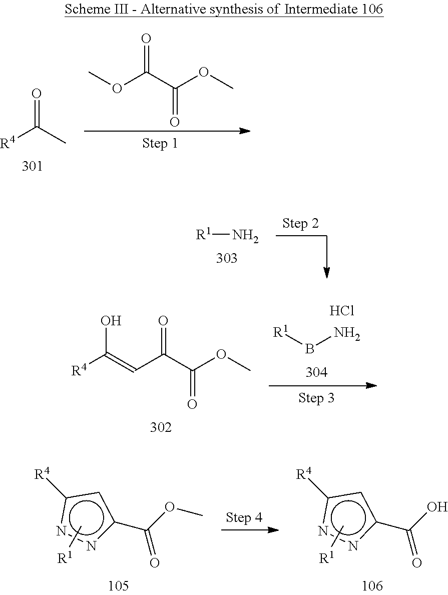

Referring to Scheme III, Step 1, to a solution of the compound of Formula 301 in a polar aprotic solvent, such as THF, is added a non-nucleophilic base, such as potassium t-butoxide, at a reduced temperature, such as 0° C. The mixture is stirred for 1-2 h. Dimethyl oxalate is added dropwise, and the mixture is stirred for at ambient temperature for 12-24 h. During this time, the progress of the reaction can be followed by chromatography, for example, TLC. The product, a compound of Formula 302, is isolated and purified using methods known in the art.

Referring to Scheme III, Step 2, to a solution of the compound of Formula 303 in an aqueous solution of HCl is added sodium nitrite in water at a reduced temperature, such as −5° C. The mixture is stirred for 1-2 h, then tin(II)chloride in HCl is added dropwise and the mixture is stirred for an additional 4-6 h. During this time, the progress of the reaction can be followed by chromatography, for example, TLC. The product, a compound of Formula 304, is used in the next step without additional purification.

Referring to Scheme III, Step 3, to a solution of the compound of Formula 302 in an organic solvent, such as ethanol, is added a compound of Formula 304 and HCl in dioxane. The mixture is stirred at elevated temperatures for 16-24 h. During this time, the progress of the reaction can be followed by chromatography, for example, TLC. The product, a compound of Formula 105, is isolated and purified using methods known in the art.

Referring to Scheme III, Step 4, the compound of Formula 105 is converted to an intermediate of Formula 106 using similar procedures as those of Scheme I, Step 3.

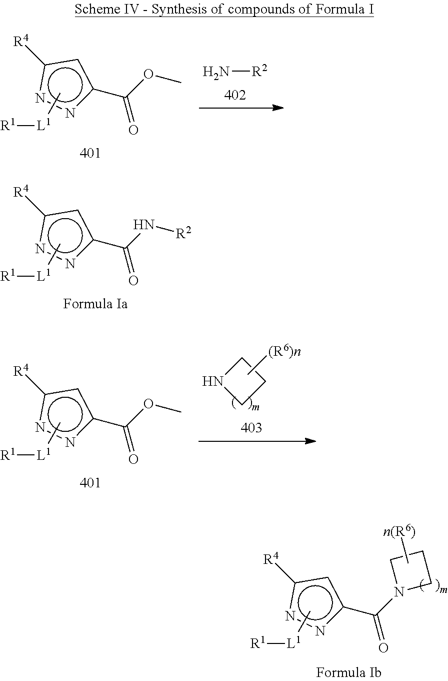

Referring to Scheme IV, Step 1, to a solution of the compound of Formula 401 in an organic solvent, such as dichloromethane, is added an activating group, such as HATU, and a non-nucleophilic base, such as diisopropylethylamine. The mixture is stirred at ambient temperature for 0.5-1 h. An amine of Formula 402 or 403 (m=1, 2, or 3; n=0, 1, or 2) is added, and the mixture is stirred for an additional 1 h. During this time, the progress of the reaction can be followed by chromatography, for example, TLC. The product, a compound of Formula Ia or Ib, is isolated and purified using methods known in the art. Individual enantiomers can be separated by using methods known in the art, such as chiral chromatography.

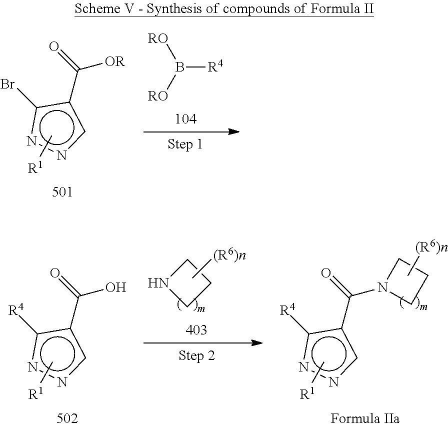

Referring to Scheme V, Step 1, to a solution of the compound of Formula 501 (R=alkyl or H) in a polar solvent, such as dioxane, is added a compound of Formula 104, a catalyst, such as Pd(dppf)Cl₂, and a base, such as potassium carbonate, in an inert atmosphere. The mixture is stirred at elevated temperature for between 1-3 h. During this time, the progress of the reaction can be followed by chromatography, for example, LCMS. If R is hydrogen, the product, a compound of Formula 502, is isolated and purified using methods known in the art. If R is alkyl, the product is subsequently dissolved in an organic solvent, such as THF, and charged with water and a strong base, such as sodium hydroxide. The mixture is stirred at ambient temperature for 1-3 h, then acidified to between pH 4-6 with a strong acid, such as HCl. During this time, the progress of the reaction can be followed by chromatography, for example, LCMS. The product, a compound of Formula 502, is isolated and purified using methods known in the art.

Referring to Scheme V, Step 2, to a solution of the compound of Formula 502 in an organic solvent, such as dichloromethane, is added an activating group, such as HATU, and a non-nucleophilic base, such as diisopropylethylamine. The mixture is stirred at ambient temperature for 0.5-1 h. An amine of Formula 403 (m=1, 2, or 3) is added, and the mixture is stirred for an additional 1 h. During this time, the progress of the reaction can be followed by chromatography, for example, TLC. The product, a compound of Formula IIa, is isolated and purified using methods known in the art. Individual enantiomers can be separated by using methods known in the art, such as chiral chromatography.

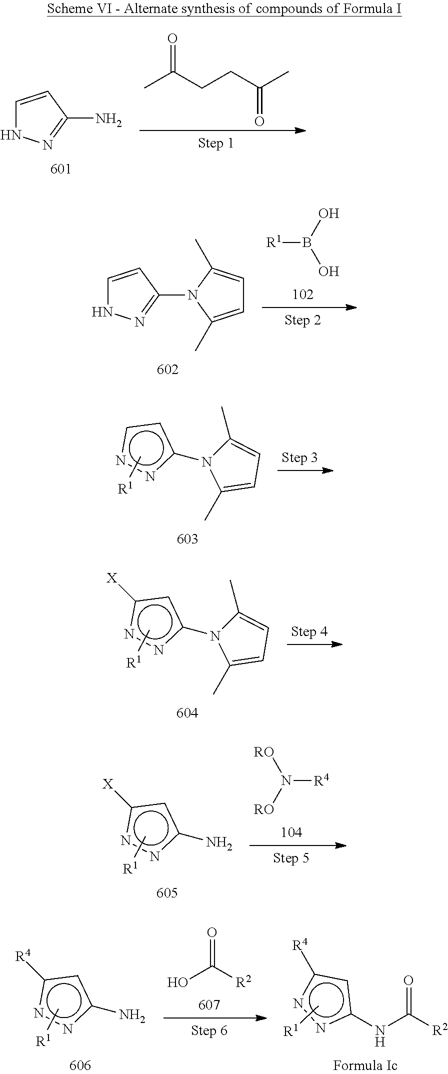

Referring to Scheme VI, Step 1, to a solution of the compound of Formula 601 in an acidic solvent, such as acetic acid, is added hexane-2,5-dione. The mixture is stirred at elevated temperature for between 10-16 h. During this time, the progress of the reaction can be followed by chromatography, for example, LCMS. The product, a compound of Formula 602, is isolated and purified using methods known in the art.

Referring to Scheme VI, Step 2, to a solution of the compound of Formula 602 in an organic solvent, such as methylene chloride, is added a compound of Formula 102 (R=alkyl, such as methyl, or hydrogen, or in combination with a second R group forms a heterocycloalkyl, such as pinacol borane), a catalyst, such as copper (II) acetate, and a base, such as triethylamine. The mixture is stirred at ambient temperature for 12-24 h. During this time, the progress of the reaction can be followed by chromatography, for example, LCMS. The product, a compound of Formula 603, is isolated and purified using methods known in the art.

Referring to Scheme VI, Step 3, to a solution of the compound of Formula 603 in a polar aprotic solvent, such as THF, is added a strong non-nucleophilic base, such as n-butyllithium, at a reduced temperature, such as −78° C. in an inert atmosphere and stirred for 0.5-1 h. A solution of a diatomic halogen, such as I₂, in a polar aprotic solvent, such as THF, is added dropwise, and the resulting mixture is stirred for an additional 1-2 h at room temperature. During this time, the progress of the reaction can be followed by chromatography, for example, LCMS. The product, a compound of Formula 604 (X=halogen, such as bromine or iodine), is isolated and purified using methods known in the art.

Referring to Scheme VI, Step 4, to a solution of the compound of Formula 604 in a polar protic solvent, such as ethanol, is added a non-nucleophilic base, such as triethylamine, and hydroxylamine. The resulting mixture is stirred at room temperature for 16-24 h at elevated temperatures. During this time, the progress of the reaction can be followed by chromatography, for example, LCMS. The product, a compound of Formula 605 (X=halogen, such as bromine or iodine), is isolated and purified using methods known in the art.

Referring to Scheme VI, Step 5, to a solution of the compound of Formula 605 in a polar solvent, such as dioxane, is added a compound of Formula 104, a catalyst, such as Pd(dppf)Cl₂, and a base, such as potassium carbonate, in an inert atmosphere. The mixture is stirred at elevated temperature for between 1-3 h. During this time, the progress of the reaction can be followed by chromatography, for example, LCMS. The product, a compound of Formula 606, is isolated and purified using methods known in the art.

Referring to Scheme VI, Step 6, to a solution of the compound of Formula 606 in an organic solvent, such as dichloromethane, is added a compound of Formula 607, an activating group, such as HATU, and a non-nucleophilic base, such as diisopropylethylamine. The mixture is stirred at ambient temperature for 0.5-1 h. During this time, the progress of the reaction can be followed by chromatography, for example, TLC. The product, a compound of Formula Ic, is isolated and purified using methods known in the art. Individual enantiomers can be separated by using methods known in the art, such as chiral chromatography.

### EXAMPLES

The following disclosure is further illustrated by the following Examples.

In the Examples below and throughout the disclosure, the following abbreviations may be used: RT or rt=Room Temperature; SM=Starting Material; MeCN or ACN=acetonitrile; AcOH=acetic acid; AcOK or KOAc=potassium acetate; DBAD=di-tert-butyl azodicarboxylate; DCM=dichloromethane; DIEA or DIPEA=N,N-Diisopropylethylamine; DMF=dimethylformamide; DMSO=dimethylsulfoxide; Et₃N or TEA=triethylamine; EtOAc=ethyl acetate; EtOH=ethanol; H₂O=water; HATU=(1-[Bis(dimethylamino)methylene]-1H-1,2,3-triazolo[4,5-b]pyridinium 3-oxide hexafluorophosphate; MeCN=acetonitrile; MeOH=methanol; n-BuLi=n-butyllithium; PE=petroleum ether; Pd(dppf)Cl₂=[1,1′-bis(diphenylphosphino)ferrocene]dichloropalladium(II); Ph₃P=triphenylphosphine; t-BuOH=t-butanol; t-BuOK=potassium t-butoxide; TFA=trifluoroacetic acid; THE=tetrahydrofuran; ¹H-NMR=Proton Nuclear magnetic Resonance; LCMS=Liquid chromatography-mass spectrometry; TLC=thin layer chromatography; HPLC=High Performance Liquid Chromatography; and Rₜ=retention time. Other abbreviations may be used and will be familiar in context to those of skill in the art.

### Example 1—Intermediates

### Intermediate A-1

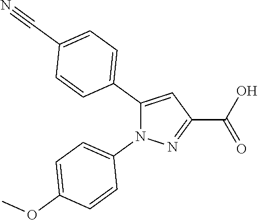 [SMILES: COc1ccc(-n2nc(C(=O)O)cc2-c2ccc(C#N)cc2)cc1]

### 5-(4-cyanophenyl)-1-(4-methoxyphenyl)-1H-pyrazole-3-carboxylic acid

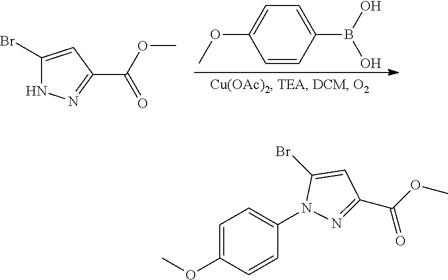

Methyl 5-bromo-1-(4-methoxyphenyl)-1H-pyrazole-3-carboxylate. To a stirred solution of methyl 5-bromo-1H-pyrazole-3-carboxylate (5.0 g, 24.5 mmol, 1.0 equiv) in DCM (100 mL) were added (4-methoxyphenyl)boronic acid (5.52 g, 36.6 mmol, 1.5 equiv), Cu(OAc)₂ (8.11 g, 36.6 mmol, 1.5 equiv) and TEA (7.51 g, 73.2 mmol, 3.0 equiv) at rt. The reaction mixture was stirred for 16 h at rt under an O₂ atmosphere. The reaction was monitored by LCMS. The resulting mixture was concentrated under vacuum. The residue was eluted from silica gel with petroleum ether/ethyl acetate (5:1). This resulted in 750 mg (9.89%) of the title compound as a red solid. MS-ESI: 311/313 (M+1).

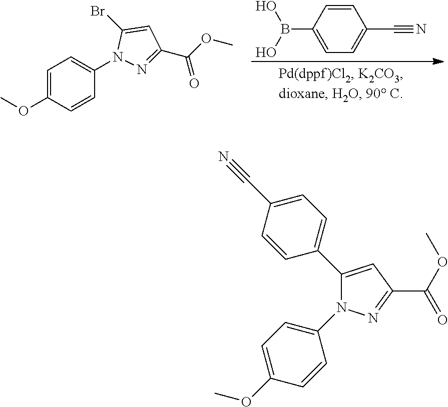

Methyl 5-(4-cyanophenyl)-1-(4-methoxyphenyl)-1H-pyrazole-3-carboxylate. To a stirred solution of methyl 5-bromo-1-(4-methoxyphenyl)-1H-pyrazole-3-carboxylate (320 mg, 1.0 mmol, 1.0 equiv) in dioxane (40 mL) and H₂O (4 mL) were added (4-cyanophenyl)boronic acid (227 mg, 1.5 mmol, 1.5 equiv), Pd(dppf)Cl₂ (151 mg, 0.2 mmol, 0.2 equiv) and K₂CO₃ (427 mg, 3.1 mmol, 3.0 equiv) under a nitrogen atmosphere. The resulting mixture was stirred for 2 h at 90° C. under a nitrogen atmosphere. The reaction was monitored by LCMS. The reaction mixture was concentrated under vacuum. The residue was eluted from silica gel with petroleum ether/ethyl acetate (3:1). This resulted in 320 mg (59.7%) of the title compound as a pink solid. MS-ESI: 334 (M+1).

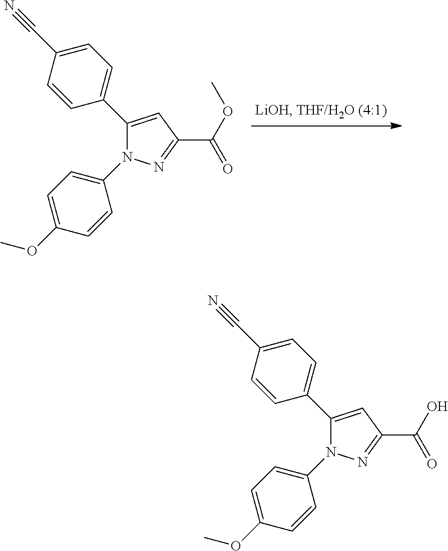 [SMILES: COC(=O)c1cc(-c2ccc(C#N)cc2)n(-c2ccc(OC)cc2)n1 ; COc1ccc(-n2nc(C(=O)O)cc2-c2ccc(C#N)cc2)cc1]

5-(4-Cyanophenyl)-1-(4-methoxyphenyl)-1H-pyrazole-3-carboxylic acid. To a stirred solution of methyl 5-(4-cyanophenyl)-1-(4-methoxyphenyl)-1H-pyrazole-3-carboxylate (320 mg, 1.0 mmol, 1.0 equiv) in THE (20 mL) and H₂O (5 mL) was added LiOH (92 mg, 3.8 mmol, 4.0 equiv). The resulting mixture was stirred for 1 h at rt. The reaction was monitored by LCMS. The mixture was acidified to pH 5 with HCl (1M). The resulting mixture was extracted with DCM (3×30 mL). The combined organic layers were dried over anhydrous Na₂SO₄ and concentrated under vacuum. This resulted in 300 mg (97.8%) of the title compound as a white solid. MS-ESI: 320 (M+1).

The intermediates in the following table were prepared using similar procedures for Intermediate A-1 from the appropriate starting materials:

TABLE 1
Pyrazol-3-carboxylates.

| Structure | Name | Int. No. | Exact Mass [M + H]⁺ |
|---|---|---|---|
| 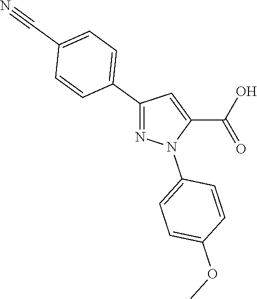 [SMILES: COc1ccc(-n2nc(-c3ccc(C#N)cc3)cc2C(=O)O)cc1] | 3-(4-Cyanophenyl)-1- (4-methoxyphenyl)-1H- pyrazole-5-carboxylic acid | A-2 | 320 |
| 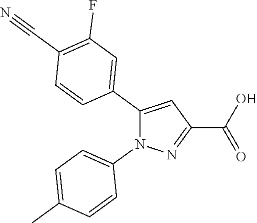 [SMILES: Cc1ccc(-n2nc(C(=O)O)cc2-c2ccc(C#N)c(F)c2)cc1] | 5-(4-Cyano-3- fluorophenyl)-1-(p- tolyl)-1H-pyrazole-3- carboxylic acid | A-3 | 322 |
| 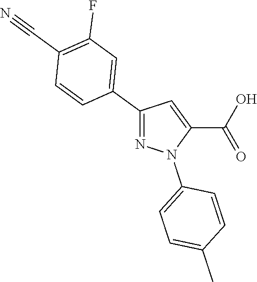 [SMILES: Cc1ccc(-n2nc(-c3ccc(C#N)c(F)c3)cc2C(=O)O)cc1] | 3-(4-Cyano-3- fluorophenyl)-1-(p- tolyl)-1H-pyrazole-5- carboxylic acid | A-4 | 322 |
| 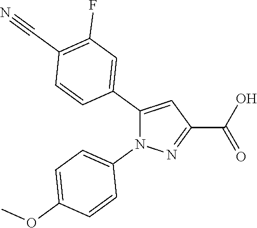 [SMILES: COc1ccc(-n2nc(C(=O)O)cc2-c2ccc(C#N)c(F)c2)cc1] | 5-(4-Cyano-3- fluorophenyl)-1-(4- methoxyphenyl)-1H- pyrazole-3-carboxylic acid | A-5 | 338 |
| 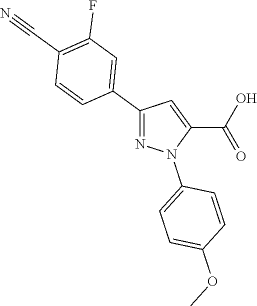 [SMILES: COc1ccc(-n2nc(-c3ccc(C#N)c(F)c3)cc2C(=O)O)cc1] | 3-(4-Cyano-3- fluorophenyl)-1-(4- methoxyphenyl)-1H- pyrazole-5-carboxylic acid | A-6 | 338 |
| 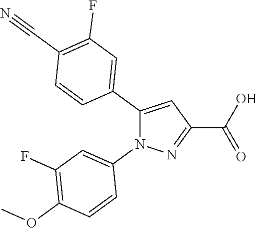 [SMILES: COc1ccc(-n2nc(C(=O)O)cc2-c2ccc(C#N)c(F)c2)cc1F] | 5-(4-Cyano-3- fluorophenyl)-1-(3- fluoro-4- methoxyphenyl)-1H- pyrazole-3-carboxylic acid | A-7 | 356 |
| 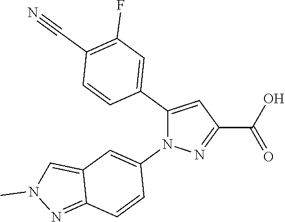 [SMILES: Cn1cc2cc(-n3nc(C(=O)O)cc3-c3ccc(C#N)c(F)c3)ccc2n1] | 5-(4-Cyano-3- fluorophenyl)-1-(2- methyl-2H-indazol-5- yl)-1H-pyrazole-3- carboxylic acid | A-8 | 362 |
| 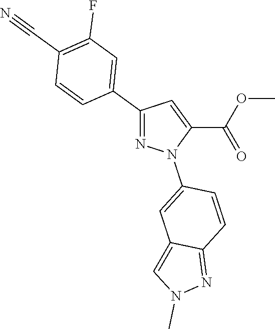 [SMILES: COC(=O)c1cc(-c2ccc(C#N)c(F)c2)nn1-c1ccc2nn(C)cc2c1] | Methyl 3-(4-Cyano-3- fluorophenyl)-1-(2- methyl-2H-indazol-5- yl)-1H-pyrazole-5- carboxylate | A-9 | 376 |
| 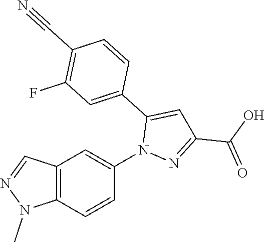 [SMILES: Cn1ncc2cc(-n3nc(C(=O)O)cc3-c3ccc(C#N)c(F)c3)ccc21] | 5-(4-Cyano-3- fluorophenyl)-1-(1- methyl-1H-indazol-5- yl)-1H-pyrazole-3- carboxylic acid | A-10 | 362 |

### Intermediate A-11

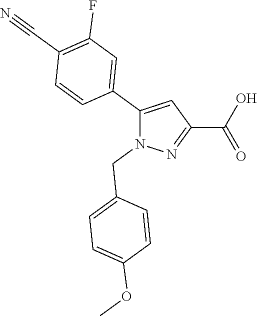 [SMILES: COc1ccc(Cn2nc(C(=O)O)cc2-c2ccc(C#N)c(F)c2)cc1]

### 5-(4-Cyano-3-fluorophenyl)-1-(4-methoxybenzyl)-1H-pyrazole-3-carboxylic acid

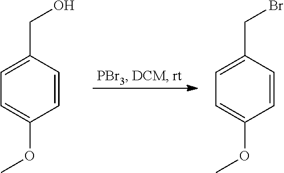

1-(Bromomethyl)-4-methoxybenzene. To a stirred solution of (4-methoxyphenyl)methanol (10 g, 72.4 mmol, 1.0 equiv) in DCM (100 mL) was added PBr₃ (39.2 g, 0.14 mmol, 2.0 equiv). The resulting solution was stirred for 1 h at 0° C. The reaction was quenched with water/ice (200 mL). The resulting solution was extracted with DCM (3×100 mL), and the combined organic layers were concentrated under vacuum. This resulted in 10 g (68.7%) of the title compound as light yellow oil.

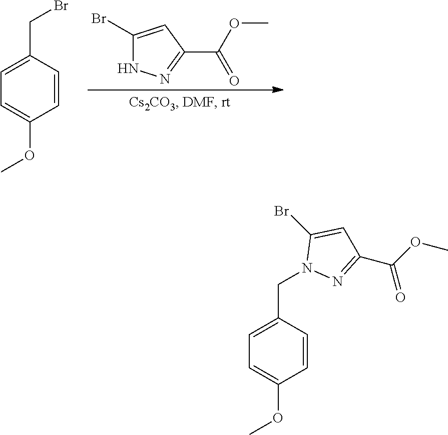

Methyl 5-bromo-1-(4-methoxybenzyl)-1H-pyrazole-3-carboxylate. To a stirred solution of methyl 5-bromo-1H-pyrazole-3-carboxylate (10 g, 48.8 mmol, 1.0 equiv) in DMF (100 mL) were added 1-(bromomethyl)-4-methoxybenzene (11.8 g, 58.6 mmol, 1.2 equiv) and Cs₂CO₃ (47.7 g, 146 mmol, 3.0 equiv). The reaction solution was stirred for 16 h at rt. The resulting solution was diluted with ethyl acetate (500 mL). The resulting mixture was washed with H₂O (5×200 mL) and brine (200 mL). The mixture was dried over anhydrous sodium sulfate and concentrated under vacuum. The crude product was purified by HP-flash with MeCN/H₂O (20-60% 30 min). This resulted in 1.2 g (7.57%) of the title compound as an off-white solid. MS-ESI: 325/327 (M+1).

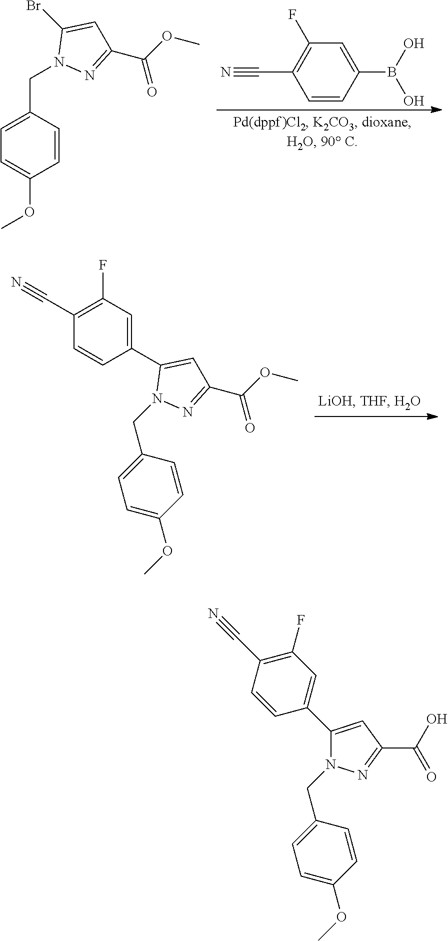

Methyl 5-bromo-1-(4-methoxybenzyl)-1H-pyrazole-3-carboxylate was converted to Intermediate A-11 using similar procedures as those used in the synthesis of Intermediate A-1.

### Intermediate A-12

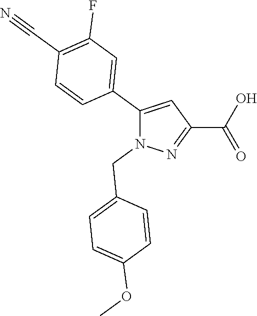 [SMILES: COc1ccc(Cn2nc(C(=O)O)cc2-c2ccc(C#N)c(F)c2)cc1]

### 5-(4-Cyano-3-fluorophenyl)-1-(2-(3-methylisoxazol-5-yl)ethyl)-1H-pyrazole-3-carboxylic acid

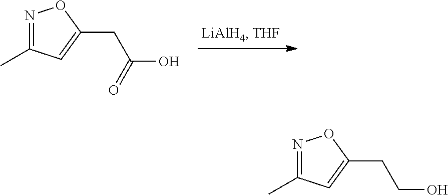

2-(3-Methylisoxazol-5-yl)ethan-1-ol To a stirred solution of 2-(3-methyl-1,2-oxazol-5-yl)acetic acid (5.0 g, 35.4 mmol, 1.0 equiv) in THF (100 mL) was added LiAlH₄ (2.69 g, 70.8 mmol, 2.0 equiv) at 0° C. The resulting mixture was stirred for 2 h at rt. The reaction was monitored by LCMS. The reaction was quenched with water (5 mL), NaOH (5 mL, 15%), and water (15 mL) at rt. The resulting mixture was filtered, and the filter cake was washed with DCM (3×100 mL). The filtrate was concentrated under vacuum. The residue was purified by reverse flash chromatography with the following conditions: Column, C18 silica gel; mobile phase, ACN in water, 5% to 25% gradient in 20 min; detector, UV 210 nm. This resulted in 2.71 g (59.9%) of the title compound as a dark yellow solid. MS-ESI: 128 (M+1).

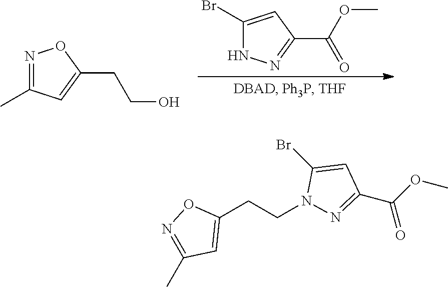

Methyl 5-bromo-1-(2-(3-methylisoxazol-5-yl)ethyl)-1H-pyrazole-3-carboxylate. To a stirred solution of 2-(3-methyl-1,2-oxazol-5-yl)ethan-1-ol (2.7 g, 21.2 mmol, 1.0 equiv) in THE (100 mL) were added methyl 5-bromo-1H-pyrazole-3-carboxylate (5.66 g, 27.6 mmol, 1.30 equiv), DBAD (9.78 g, 42.5 mmol, 2.0 equiv), and Ph₃P (8.35 g, 31.8 mmol, 1.5 equiv) at rt. The resulting mixture was stirred for 2 h at rt under a nitrogen atmosphere. The reaction was monitored by LCMS. The resulting mixture was concentrated under vacuum. The residue was eluted from silica gel with petroleum ether/ethyl acetate (7:1). This resulted in 2.11 g (31.5%) of the title compound as a yellow oil. MS-ESI: 314 (M+1).

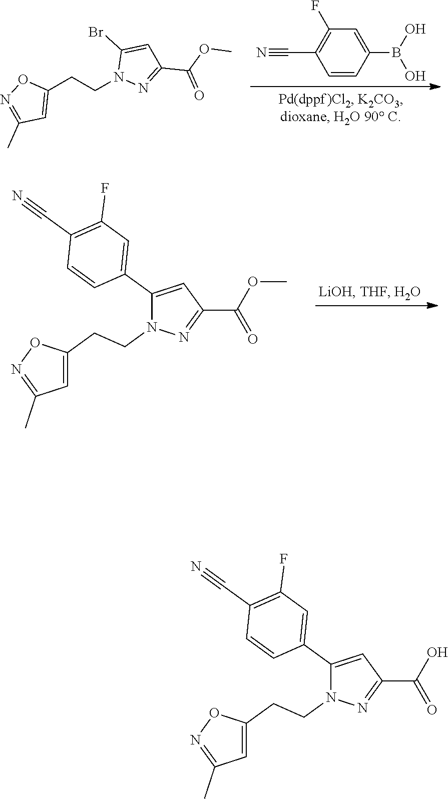

Methyl 5-bromo-1-(2-(3-methylisoxazol-5-yl)ethyl)-1H-pyrazole-3-carboxylate was converted to Intermediate A-12 using similar procedures as those used in the synthesis of Intermediate A-1.

### Intermediate A-13

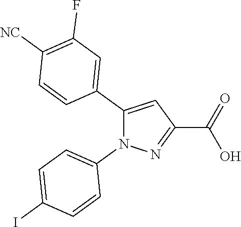 [SMILES: N#Cc1ccc(-c2cc(C(=O)O)nn2-c2ccc(I)cc2)cc1F]

### 5-(4-Cyano-3-fluorophenyl)-1-(4-iodophenyl)-1H-pyrazole-3-carboxylic acid

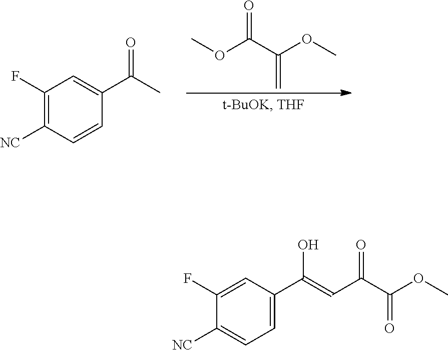

Methyl (Z)-4-(4-cyano-3-fluorophenyl)-4-hydroxy-2-oxobut-3-enoate. A solution of t-BuOK (8.05 g, 71.7 mmol, 1.3 equiv) in THE (200 mL) was added 4-acetyl-2-fluorobenzonitrile (9.0 g, 55.2 mmol, 1.0 equiv) dropwise at 0° C. under nitrogen. The resulting mixture was stirred for 1 h at 0° C. under nitrogen. To above mixture was added dimethyl oxalate (7.82 g, 66.2 mmol, 1.2 equiv) dropwise over 10 min at 0° C. The resulting mixture was stirred overnight at rt. The precipitated solids were collected by filtration and washed with THE (10 mL). The residue was dissolved in water (100 mL) and adjusted to pH 3-4 with HCl (2 M). The precipitated solids were collected by filtration and washed with water (10 mL) to afford the title compound (8.0 g, 49%) as a yellow solid. MS-ESI: 250 (M+1).

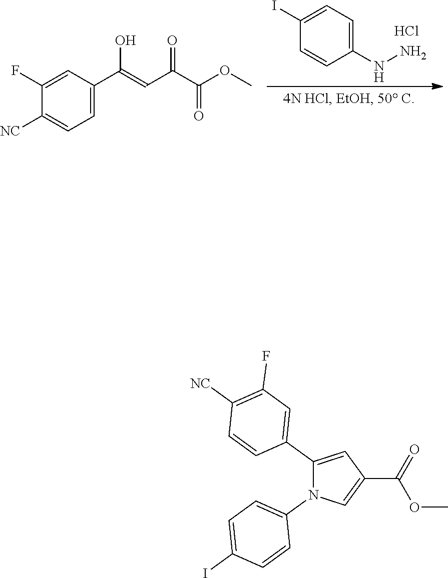 [SMILES: COC(=O)C(=O)/C=C(\O)c1ccc(C#N)c(F)c1 ; NNc1ccc(I)cc1 ; Cl ; COC(=O)c1cc(-c2ccc(C#N)c(F)c2)n(-c2ccc(I)cc2)c1]

Methyl 5-(4-cyano-3-fluorophenyl)-1-(4-iodophenyl)-1H-pyrazole-3-carboxylate. A solution of methyl (Z)-4-(4-cyano-3-fluorophenyl)-4-hydroxy-2-oxobut-3-enoate (5.0 g, 20 mmol, 1.0 equiv) in EtOH (100 mL) were added (4-iodophenyl)hydrazine (4.7 g, 20 mmol, 1.0 equiv) and HCl (gas) in 1,4-dioxane (4 M, 5.02 mL, 20 mmol, 1.0 equiv) dropwise. The resulting mixture was stirred 16 h at 50° C. and concentrated under vacuum. The residue was eluted from silica gel with petroleum ether/ethyl acetate (5:1) to afford the title compound (900 mg, 8.42%) as a yellow solid. MS-ESI: 448 (M+1).

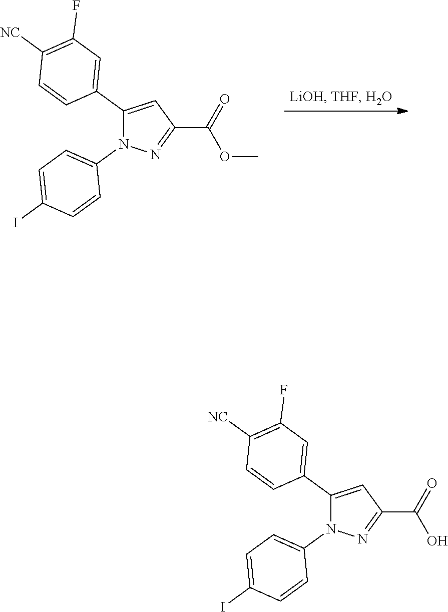 [SMILES: COC(=O)c1cc(-c2ccc(C#N)c(F)c2)n(-c2ccc(I)cc2)n1 ; N#Cc1ccc(-c2cc(C(=O)O)nn2-c2ccc(I)cc2)cc1F]

5-(4-Cyano-3-fluorophenyl)-1-(4-iodophenyl)-1H-pyrazole-3-carboxylic acid. To a stirred solution of Methyl 5-(4-cyano-3-fluorophenyl)-1-(4-iodophenyl)-1H-pyrazole-3-carboxylate (575 mg, 1.3 mmol, 1.0 equiv) in THF (6 mL) and H₂O (3 mL) was added LiOH (46.2 mg, 1.9 mmol, 1.5 equiv). The resulting mixture was stirred for 1 h at rt. The residue was adjusted to pH 3-4 with HCl (2M). The resulting mixture was extracted with ethyl acetate (3×20 mL). The combined organic layers were dried with anhydrous Na₂SO₄ and concentrated under vacuum. This resulted in 560 mg (90%) of the title compound as a brown solid. MS-ESI: 434 (M+1).

The intermediates in the following table were prepared using similar procedures for Intermediate A-13 from the appropriate starting materials:

TABLE 2
Pyrazol-3-carboxylates.

| Structure | Name | Int. No. | Exact Mass [M + H]⁺ |
|---|---|---|---|
| 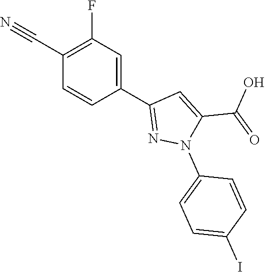 [SMILES: N#Cc1ccc(-c2cc(C(=O)O)n(-c3ccc(I)cc3)n2)cc1F] | 3-(4-Cyano-3- fluorophenyl)-1-(4- iodophenyl)-1H- pyrazole-5-carboxylic acid | A-14 | 434 |
| 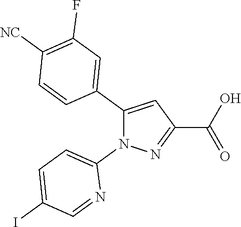 [SMILES: N#Cc1ccc(-c2cc(C(=O)O)nn2-c2ccc(I)cn2)cc1F] | 5-(4-Cyano-3- fluorophenyl)-1-(5- iodopyridin-2-yl)-1H- pyrazole-3-carboxylic acid | A-15 | 435 |
| 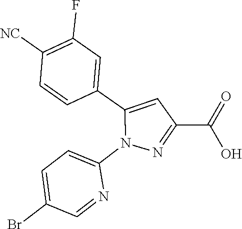 [SMILES: N#Cc1ccc(-c2cc(C(=O)O)nn2-c2ccc(Br)cn2)cc1F] | 1-(5-Bromopyridin-2- yl)-5-(4-cyano-3- fluorophenyl)-1H- pyrazole-3-carboxylic acid | A-16 | 387/389 |

### Intermediate A-17

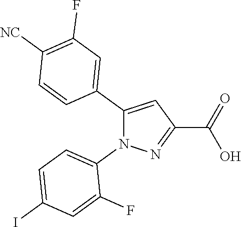 [SMILES: N#Cc1ccc(-c2cc(C(=O)O)nn2-c2ccc(I)cc2F)cc1F]

### 5-(4-Cyano-3-fluorophenyl)-1-(4-iodophenyl)-1H-pyrazole-3-carboxylic acid

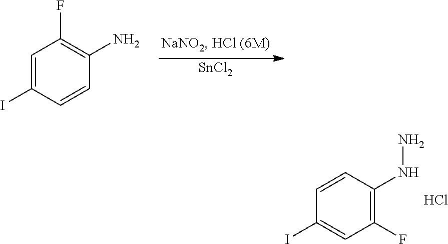 [SMILES: Nc1ccc(I)cc1F ; "SnCl2" ; NNc1ccc(I)cc1F ; Cl]

(2-Fluoro-4-iodophenyl)hydrazine hydrochloride. To a stirred mixture of 2-fluoro-4-iodoaniline (10 g, 42.2 mmol, 1.0 equiv) in HCl (6 M, 110 mL) were added sodium nitrite (3.2 g, 46.4 mmol, 1.1 equiv) in H₂O (5 mL) dropwise at −5° C. The resulting mixture was stirred for 1 h at −5° C. To above reaction mixture was added SnCl₂ (20.2 g, 105 mmol, 2.5 equiv) in cc. HCl (8 mL) dropwise at −10° C. The resulting mixture was stirred for 5 h at −5° C. The precipitated solids were collected by filtration and washed with DCM (3×30 mL). This resulted in 17.8 g (crude) of the title compound as a yellow solid. 289 (M+1).

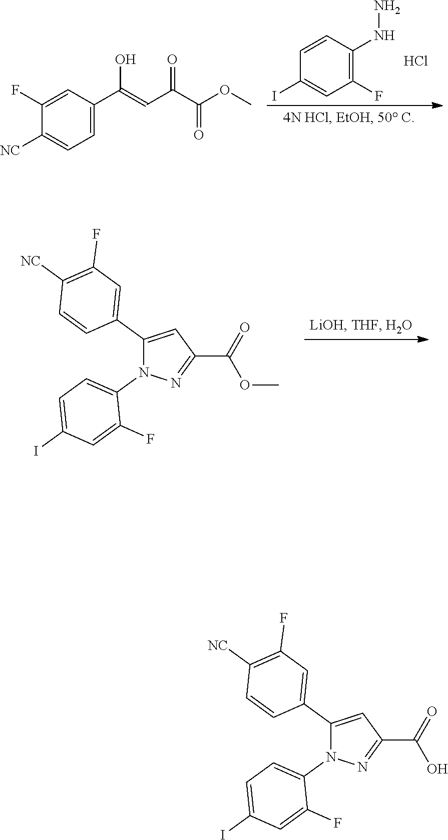

(2-Fluoro-4-iodophenyl)hydrazine hydrochloride was converted to Intermediate A-17 using similar procedures as those used in the synthesis of Intermediate A-13.

The intermediate in the following table was prepared using similar procedures for Intermediate A-17 from the appropriate starting materials:

TABLE 3
Pyrazol-3-carboxylates.

| Structure | Name | Int. No. | Exact Mass [M + H]⁺ |
|---|---|---|---|
| 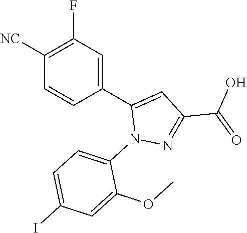 [SMILES: COc1cc(I)ccc1-n1nc(C(=O)O)cc1-c1ccc(C#N)c(F)c1] | 5-(4-Cyano-3- fluorophenyl)-1-(4- iodo-2- methoxyphenyl)-1H- pyrazole-3-carboxylic acid | A-18 | 464 |

### Intermediate A-19

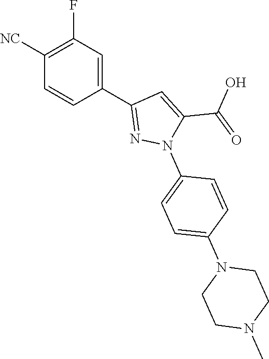 [SMILES: CN1CCN(c2ccc(-n3nc(-c4ccc(C#N)c(F)c4)cc3C(=O)O)cc2)CC1]

### 3-(4-Cyano-3-fluorophenyl)-1-(4-(4-methylpiperazin-1-yl)phenyl)-1H-pyrazole-5-carboxylic acid

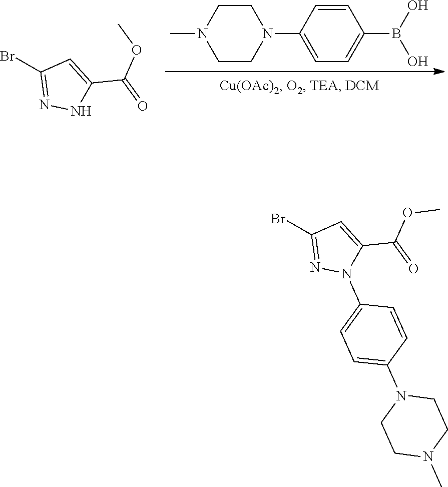

Methyl 3-bromo-1-(4-(4-methylpiperazin-1-yl)phenyl)-1H-pyrazole-5-carboxylate. To a stirred solution of methyl 3-bromo-1H-pyrazole-5-carboxylate (12 g, 58.5 mmol, 1.0 equiv) in DCM (120 mL) were added (4-(4-methylpiperazin-1-yl)phenyl)boronic acid (12.9 g, 58.5 mmol, 1.0 equiv), Cu(OAc)₂ (16 g, 87.8 mmol, 1.5 equiv) and TEA (17.8 g, 176 mmol, 3.0 equiv) at room temperature under oxygen atmosphere. The resulting mixture was stirred for 20 h at room temperature under oxygen atmosphere. The resulting mixture was filtered, and the filter cake was washed with DCM (3×100 mL). The filtrate was concentrated under vacuum. The residue was eluted from silica gel with DCM/MeOH (10:1). The isomers were separated by Prep-HPLC with the following condition: Prep-HPLC with the following conditions: XB C18 Column, 50*250 mm, 10 m; Mobile Phase A: water (10 mM NH₄HCO₃), Mobile Phase B: ACN; Flow rate: 95 mL/min; Gradient: 35% B to 55% B in 30 min, Detector UV: 210/254 nm; Rₜ 1 (min): 15.1. This resulted in 3.7 g (16.6%) of the title compound as a brown solid. MS-ESI: 379/381 (M+1). ¹H NMR (400 MHz, DMSO-d₆) δ 7.27 (d, J=8.0 Hz, 2H), 7.19 (s, 1H), 6.98 (d, J=8.0 Hz, 2H), 3.73 (s, 3H), 3.22 (t, J=6.0 Hz, 4H), 2.46 (t, J=6.0 Hz, 4H), 2.33 (s, 3H).

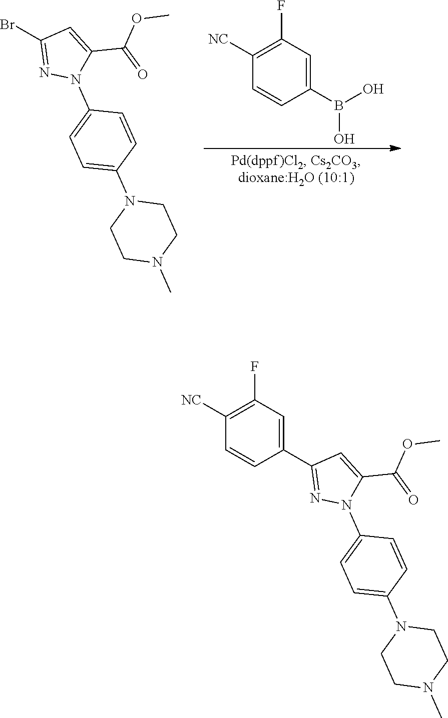 [SMILES: COC(=O)c1cc(Br)nn1-c1ccc(N2CCN(C)CC2)cc1 ; N#Cc1ccc(B(O)O)cc1F ; COC(=O)c1cc(-c2ccc(C#N)c(F)c2)nn1-c1ccc(N2CCN(C)CC2)cc1]

Methyl 3-(4-cyano-3-fluorophenyl)-1-(4-(4-methylpiperazin-1-yl)phenyl)-1H-pyrazole-5-carboxylate. To a stirred solution of methyl 3-bromo-1-(4-(4-methylpiperazin-1-yl)phenyl)-1H-pyrazole-5-carboxylate (3.5 g, 9.23 mmol, 1.0 equiv) in dioxane (30 mL) and H₂O (3 mL) were added (4-cyano-3-fluorophenyl)boronic acid (3.04 g, 18.5 mmol, 2.0 equiv), Pd(dppf)Cl₂·DCM (1.5 g, 1.85 mmol, 0.20 equiv) and Cs₂CO₃ (7.52 g, 23.1 mmol, 2.5 equiv) at room temperature under a nitrogen atmosphere. The resulting mixture was stirred overnight at 90° C. under a nitrogen atmosphere. The mixture was allowed to cool down to room temperature. The resulting mixture was filtered, and the filter cake was washed with DCM (3×100 mL). The filtrate was concentrated under vacuum. The residue was eluted from silica gel with DCM/MeOH (10:1). This resulted in 3.0 g (77.5%) of the title compound as a brown solid. MS-ESI: 420 (M+1).

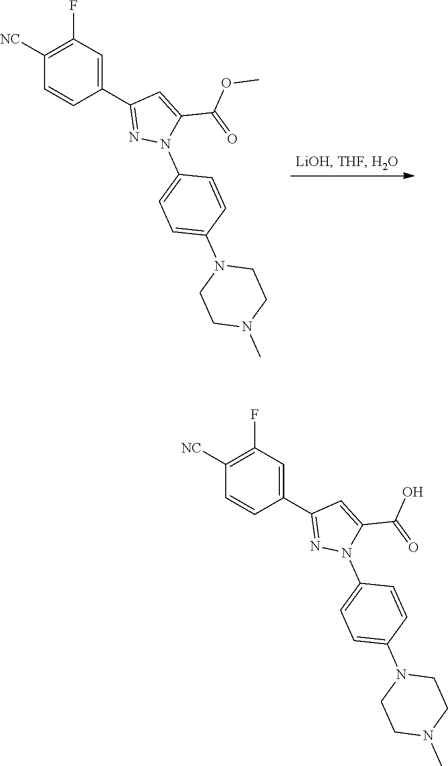 [SMILES: COC(=O)c1cc(-c2ccc(C#N)c(F)c2)nn1-c1ccc(N2CCN(C)CC2)cc1 ; CN1CCN(c2ccc(-n3nc(-c4ccc(C#N)c(F)c4)cc3C(=O)O)cc2)CC1]

3-(4-Cyano-3-fluorophenyl)-1-(4-(4-methylpiperazin-1-yl)phenyl)-1H-pyrazole-5-carboxylic acid. To a stirred solution of methyl 3-(4-cyano-3-fluorophenyl)-1-(4-(4-methylpiperazin-1-yl)phenyl)-1H-pyrazole-5-carboxylate (3.0 g, 7.15 mmol, 1.0 equiv) in THF (20 mL) and H₂O (4 mL) was added LiOH (0.34 g, 14.3 mmol, 2.0 equiv) at room temperature. The resulting mixture was stirred for 1.5 h at room temperature. The mixture was acidified to pH 3 with HCl (2M) and extracted with DCM (3×100 mL). The combined organic layers were dried over anhydrous Na₂SO₄ and concentrated under vacuum. This resulted in 2.0 g (68.9%) of the title compound as a brown solid. MS-ESI: 406 (M+1).

### Intermediate A-20

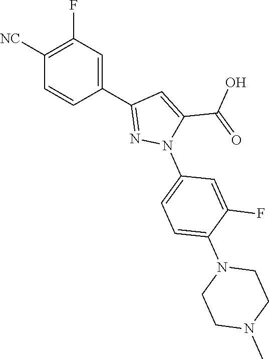 [SMILES: CN1CCN(c2ccc(-n3nc(-c4ccc(C#N)c(F)c4)cc3C(=O)O)cc2F)CC1]

### 3-(4-Cyano-3-fluorophenyl)-1-(3-fluoro-4-(4-methylpiperazin-1-yl)phenyl)-1H-pyrazole-5-carboxylic acid

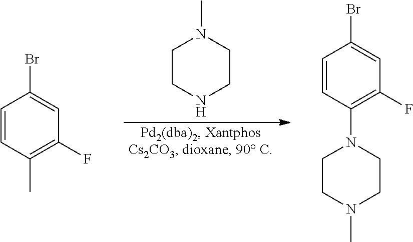

1-(4-Bromo-2-fluorophenyl)-4-methylpiperazine. To a stirred mixture of 4-bromo-2-fluoro-1-iodobenzene (50 g, 166 mmol, 1.0 equiv) in dioxane (1 L) were added 1-methylpiperazine (16.6 g, 166 mmol, 1.0 equiv), Pd₂(dba)₃ (15.2 g, 16.6 mmol, 0.1 equiv), XantPhos (15.8 g, 16.6 mmol, 0.1 equiv), and Cs₂CO₃ (162 g, 499 mmol, 3.0 equiv) under a nitrogen atmosphere. The resulting mixture was stirred overnight at 100° C. under a nitrogen atmosphere. The mixture was allowed to cool down to room temperature. The reaction mixture was added water (500 mL) and extracted with DCM (3×500 mL). The combined organic layers were dried over anhydrous Na₂SO₄ and concentrated under vacuum. The residue was eluted from silica gel with PE/EtOAc (1:1). This resulted in 10 g (19.8%) of the title compound as a yellow solid. MS-ESI: 273/275 (M+1).

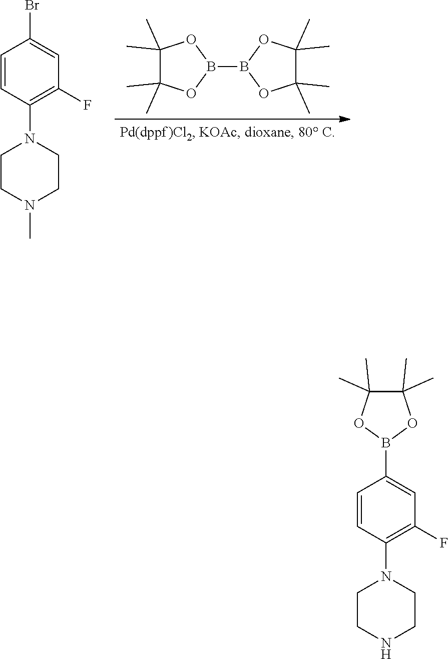

1-(2-Fluoro-4-(4,4,5,5-tetramethyl-1,3,2-dioxaborolan-2-yl)phenyl)piperazine. To a stirred mixture of 1-(4-bromo-2-fluorophenyl)-4-methylpiperazine (10 g, 36.6 mmol, 1.0 equiv) in dioxane (300 mL) were added bis(pinacolato)diboron (14 g, 54.9 mmol, 1.5 equiv), Pd(dppf)Cl₂·DCM (2.98 g, 3.66 mmol, 0.1 equiv), and AcOK (14.4 g, 146 mmol, 4.0 equiv). The resulting mixture was stirred overnight at 90° C. under a nitrogen atmosphere. The mixture was allowed to cool down to room temperature. The reaction mixture was added water (300 mL) and extracted with DCM (3×350 mL). The combined organic layers were dried over anhydrous Na₂SO₄ and concentrated under vacuum. The residue was eluted from silica gel with DCM/MeOH (10:1). This resulted in 5.5 g (42.2%) of the title compound as a yellow solid. MS-ESI: 307 (M+1).

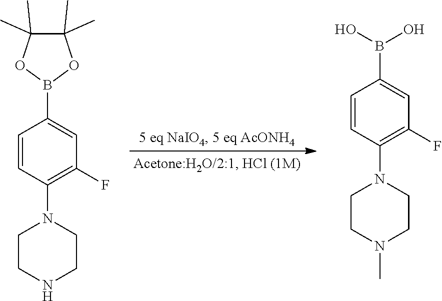 [SMILES: CC1(C)OB(c2ccc(N3CCNCC3)c(F)c2)OC1(C)C ; CN1CCN(c2ccc(B(O)O)cc2F)CC1]

(3-Fluoro-4-(4-methylpiperazin-1-yl)phenyl)boronic acid. To a stirred solution of 1-(2-fluoro-4-(4,4,5,5-tetramethyl-1,3,2-dioxaborolan-2-yl) phenyl)-4-methylpiperazine (5.5 g, 17.2 mmol, 1.0 equiv) in Acetate (250 mL) and H₂O (125 mL) were added NaIO₄ (18.4 g, 85.9 mmol, 5.0 equiv) and NH₄OAc (6.62 g, 85.9 mmol, 5.0 equiv). The reaction mixture was stirred for 2 h at room temperature. The resulting mixture was filtered, and the filter cake was washed with EtOAc (2×300 mL). The filtrate was concentrated under vacuum. The residue was purified by reversed-phase flash chromatography with the following conditions: C18; mobile phase, MeCN in Water (10 mM NH₄HCO₃), 10% to 25% in 30 min; Detector, UV 254 nm. This resulted in 1.75 g (40.6%) of the title compound as a yellow solid. MS-ESI: 239 (M+1).

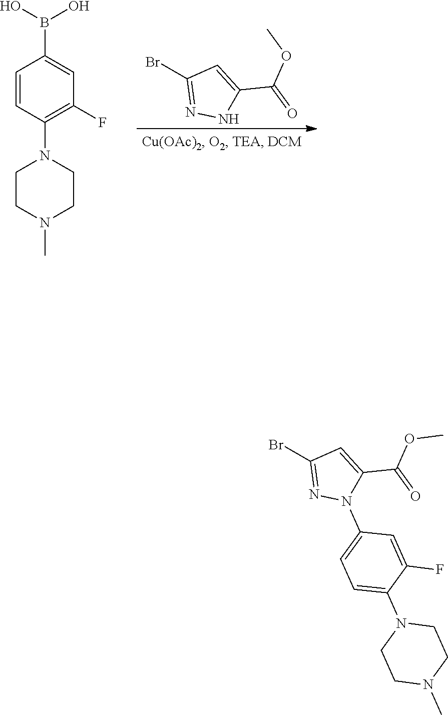

Methyl 3-bromo-1-(3-fluoro-4-(4-methylpiperazin-1-yl)phenyl)-1H-pyrazole-5-carboxylate. To a stirred solution of (3-fluoro-4-(4-methylpiperazin-1-yl) phenyl) boronic acid (1.75 g, 7.35 mmol, 1.0 equiv) in DCM (40 mL) were added methyl 3-bromo-1H-pyrazole-5-carboxylate (1.96 g, 9.56 mmol, 1.3 equiv), Cu(OAc)₂ (4.01 g, 22.1 mmol, 3.0 equiv), and TEA (2.23 g, 22.1 mmol, 3.0 equiv). The reaction mixture was stirred overnight under oxygen atmosphere. The reaction mixture was added water (30 mL) and extracted with DCM (3×40 mL). The combined organic layers were dried over anhydrous Na₂SO₄ and concentrated under vacuum. The residue was purified by reversed-phase flash chromatography with the following conditions: C18; mobile phase, MeCN in Water (10 mM NH₄HCO₃), 20% to 80% in 45 min; Detector UV, 254 nm. This resulted in 1.05 g (34.1%) of the title compound as a white solid. MS-ESI: 397/399 (M+1). ¹H NMR (400 MHz, CDCl₃-d₁) δ 7.20-7.07 (m, 2H), 7.02-6.89 (m, 2H), 3.81 (s, 3H), 3.18 (s, 4H), 2.62 (s, 4H), 2.37 (s, 3H).

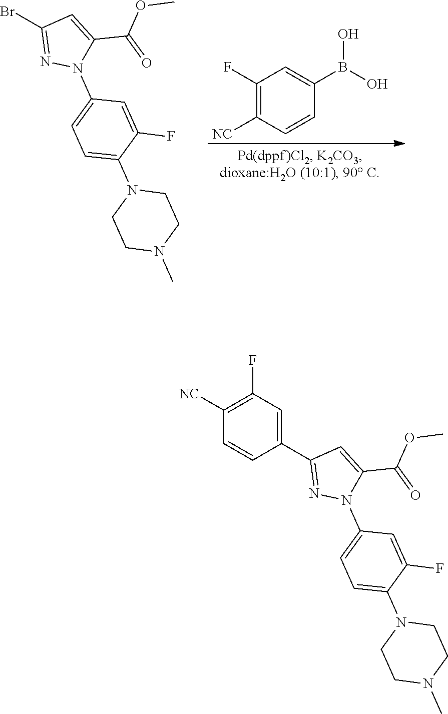 [SMILES: COC(=O)c1cc(Br)nn1-c1ccc(N2CCN(C)CC2)c(F)c1 ; N#Cc1ccc(B(O)O)cc1F ; COC(=O)c1cc(-c2ccc(C#N)c(F)c2)nn1-c1ccc(N2CCN(C)CC2)c(F)c1]

Methyl 3-(4-cyano-3-fluorophenyl)-1-(3-fluoro-4-(4-methylpiperazin-1-yl)phenyl)-1H-pyrazole-5-carboxylate. To a stirred methyl 3-bromo-1-(3-fluoro-4-(4-methylpiperazin-1-yl) phenyl)-1H-pyrazole-5-carboxylate (1.0 g, 2.52 mmol, 1.0 equiv) in dioxane (40 mL) were added 4-cyano-3-fluorophenylboronic acid (0.50 g, 3.02 mmol, 1.2 equiv), Pd(dppf)Cl₂·DCM (0.21 g, 0.25 mmol, 0.1 equiv), and Cs₂CO₃ (2.46 g, 7.55 mmol, 3.0 equiv) under a nitrogen atmosphere. The resulting mixture was stirred overnight at 100° C. under a nitrogen atmosphere. The mixture was allowed to cool down to room temperature. The reaction mixture was added water (40 mL) and extracted with DCM (3×40 mL). The combined organic layers were dried over anhydrous Na₂SO₄ and concentrated under vacuum. The residue was eluted from silica gel with MeOH/DCM (1:10). This resulted in 500 mg (45.4%) of the title compound as a yellow solid. MS-ESI: 438 (M+1).

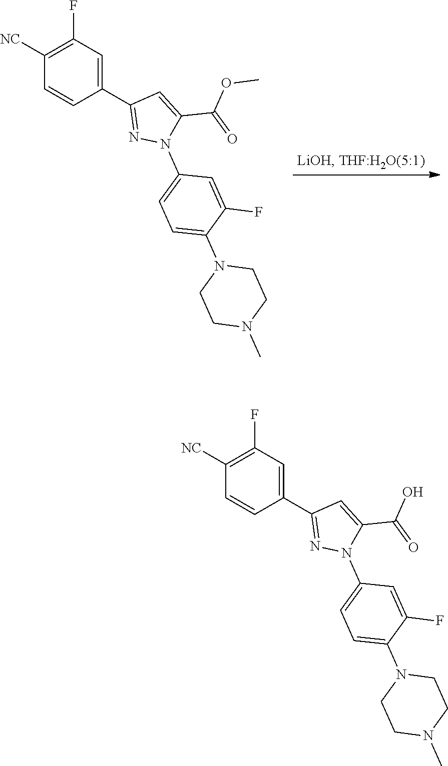 [SMILES: COC(=O)c1cc(-c2ccc(C#N)c(F)c2)nn1-c1ccc(N2CCN(C)CC2)c(F)c1 ; CN1CCN(c2ccc(-n3nc(-c4ccc(C#N)c(F)c4)cc3C(=O)O)cc2F)CC1]

3-(4-Cyano-3-fluorophenyl)-1-(3-fluoro-4-(4-methylpiperazin-1-yl)phenyl)-1H-pyrazole-5-carboxylic acid. To a stirred solution of methyl 3-(4-cyano-3-fluorophenyl)-1-(3-fluoro-4-(4-methylpiperazin-1-yl) phenyl)-1H-pyrazole-5-carboxylate (500 mg, 1.14 mmol, 1.0 equiv) in THE (25 mL) and H₂O (5 mL) was added LiOH (80 mg, 3.43 mmol, 3.0 equiv) at room temperature. The resulting mixture was stirred for 2 h at room temperature. The mixture was acidified to pH 5 with HCl (2M) and concentrated under vacuum. This resulted in 600 mg (crude) of the title compound as yellow solid. MS-ESI: 424 (M+1).

### Example 2

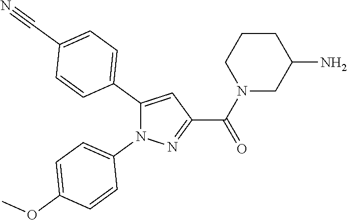 [SMILES: COc1ccc(-n2nc(C(=O)N3CCCC(N)C3)cc2-c2ccc(C#N)cc2)cc1]

### 4-(3-(3-Aminopiperidine-1-carbonyl)-1-(4-methoxyphenyl)-1H-pyrazol-5-yl)benzonitrile

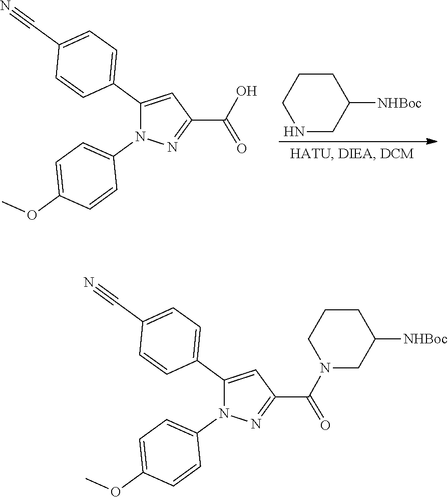

Tert-butyl (1-(5-(4-cyanophenyl)-1-(4-methoxyphenyl)-1H-pyrazole-3-carbonyl)piperidin-3-yl)carbamate. To a stirred solution of Intermediate A-1 (300 mg, 0.9 mmol, 1.0 equiv) in DCM (30 mL) were added HATU (536 mg, 1.4 mmol, 1.5 equiv) and DIEA (365 mg, 2.8 mmol, 3.0 equiv) at rt. The resulting mixture was stirred for 45 min at rt. To the above mixture was added tert-butyl N-(piperidin-3-yl)carbamate (283 mg, 1.4 mmol, 1.5 equiv) and stirred for additional 1 h at rt. The reaction was monitored by LCMS. The resulting mixture was concentrated under vacuum. The residue was eluted from silica gel with petroleum ether/ethyl acetate (1:1). This resulted in 270 mg (57.3%) of the title compound as a white solid. MS-ESI: 502 (M+1).

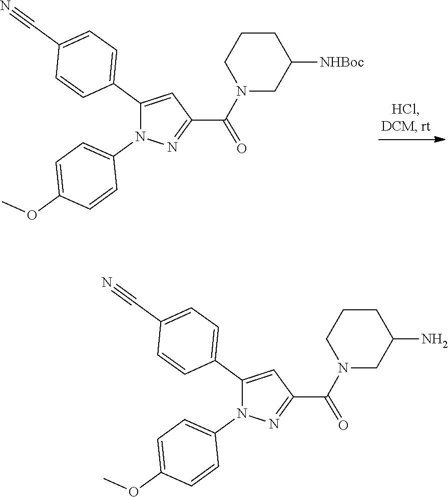 [SMILES: COc1ccc(-n2nc(C(=O)N3CCCC(NC(=O)OC(C)(C)C)C3)cc2-c2ccc(C#N)cc2)cc1 ; COc1ccc(-n2nc(C(=O)N3CCCC(N)C3)cc2-c2ccc(C#N)cc2)cc1]

4-(3-(3-Aminopiperidine-1-carbonyl)-1-(4-methoxyphenyl)-1H-pyrazol-5-yl)benzonitrile. A solution of tert-butyl (1-(5-(4-cyanophenyl)-1-(4-methoxyphenyl)-1H-pyrazole-3-carbonyl)piperidin-3-yl)carbamate (200 mg, 0.4 mmol, 1.0 equiv) in HCl in dioxane (4M, 10 mL) was stirred for 1 h at rt. The reaction mixture was stirred for 45 min at rt. The reaction was monitored by LCMS. The mixture was basified to pH 7 with saturated Na₂CO₃ (aq.). The crude product (170 mg) was purified by Prep-HPLC with the following conditions: Column: XBridge Prep OBD C18 Column 30 mm, 150 mm 5 μm; Mobile Phase A: water (10 mM NH₄HCO₃+0.1% NH₃·H₂O), Mobile Phase B: ACN; Flow rate: 60 mL/min; Gradient: 37% B to 55% B in 7 min; 254/220 nm; Rₜ: 6.18 min. This resulted in 40 mg (24%) of the title compound as a white solid. MS-ESI: 402 (M+1). ¹H NMR (400 MHz, DMSO-d₆) δ 7.84 (d, J=7.6 Hz, 2H), 7.45 (d, J=8.4 Hz, 2H), 7.27 (t, J=8.0 Hz, 2H), 7.02 (t, J=8.4 Hz, 3H), 4.43-4.17 (m, 2H), 3.79 (s, 3H), 3.09 (t, J=12.2 Hz, 1H), 2.97-2.86 (m, 1H), 2.67-2.60 (m, 1H), 1.88-1.66 (m, 3H), 1.41 (s, 1H), 1.30 (s, 1H).

Examples in the following table were prepared using similar conditions as described for those of Example 2 from the appropriate starting materials.

TABLE 4
Example Compounds.

| Ex. | | | |
|---|---|---|---|
| No. | Structure | Name | Data |
| 3 | 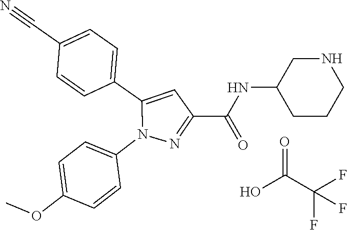 | 5-(4-Cyanophenyl)-1-(4- methoxyphenyl)-N- (piperidin-3-yl)-1H- pyrazole-3-carboxamide 2,2,2-trifluoroacetate | MS-ESI: 402 (M + 1) |
| 4 | 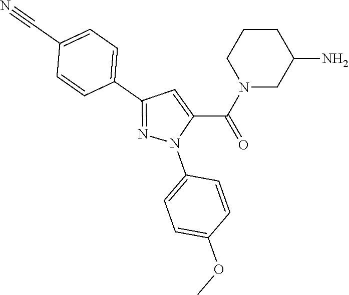 [SMILES: COc1ccc(-n2nc(-c3ccc(C#N)cc3)cc2C(=O)N2CCCC(N)C2)cc1] | 4-(5-(3-Aminopiperidine- 1-carbonyl)-1-(4- methoxyphenyl)-1H- pyrazol-3-yl)benzonitrile | MS-ESI: 402 (M + 1) |
| 5 | 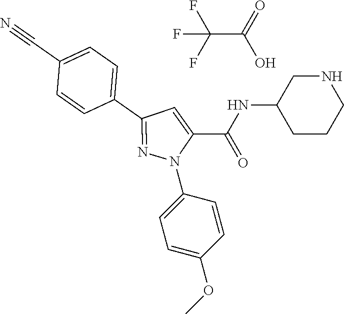 | 3-(4-Cyanophenyl)-1-(4- methoxyphenyl)-N- (piperidin-3-yl)-1H- pyrazole-5-carboxamide 2,2,2-trifluoroacetate | MS-ESI: 402 (M + 1) |
| 6 | 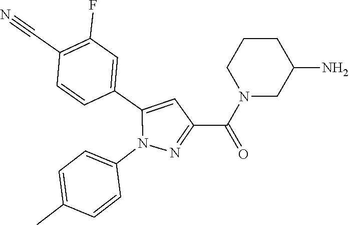 [SMILES: Cc1ccc(-n2nc(C(=O)N3CCCC(N)C3)cc2-c2ccc(C#N)c(F)c2)cc1] | 4-(3-(3-Aminopiperidine- 1-carbonyl)-1-(p-tolyl)- 1H-pyrazol-5-yl)-2- fluorobenzonitrile | MS-ESI: 404 (M + 1) |
| 7 | 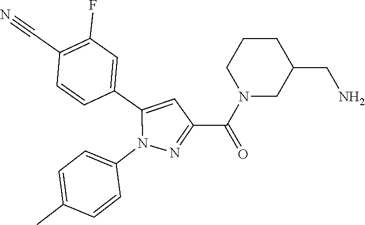 [SMILES: Cc1ccc(-n2nc(C(=O)N3CCCC(CN)C3)cc2-c2ccc(C#N)c(F)c2)cc1] | 4-(3-(3- (Aminomethyl)piperidine- 1-carbonyl)-1-(p-tolyl)- 1H-pyrazol-5-yl)-2- fluorobenzonitrile | MS-ESI: 418 (M + 1) |
| 8 | 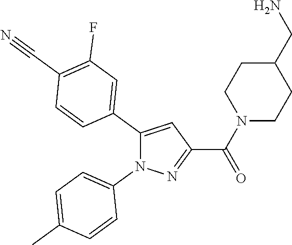 [SMILES: Cc1ccc(-n2nc(C(=O)N3CCC(CN)CC3)cc2-c2ccc(C#N)c(F)c2)cc1] | 4-(3-(4- (Aminomethyl)piperidine- 1-carbonyl)-1-(p-tolyl)- 1H-pyrazol-5-yl)-2- fluorobenzonitrile | MS-ESI: 418 (M + 1) |
| 9 | 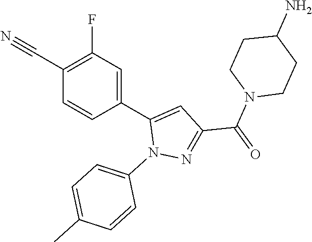 [SMILES: Cc1ccc(-n2nc(C(=O)N3CCC(N)CC3)cc2-c2ccc(C#N)c(F)c2)cc1] | 4-(3-(4-Aminopiperidine- 1-carbonyl)-1-(p-tolyl)- 1H-pyrazol-5-yl)-2- fluorobenzonitrile | MS-ESI: 404 (M + 1) |
| 10 | 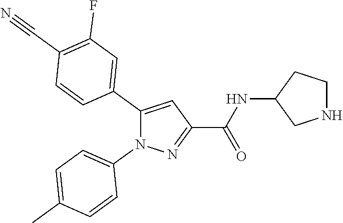 [SMILES: Cc1ccc(-n2nc(C(=O)NC3CCNC3)cc2-c2ccc(C#N)c(F)c2)cc1] | 5-(4-Cyano-3- fluorophenyl)-N- (pyrrolidin-3-yl)-1-(p- tolyl)-1H-pyrazole-3- carboxamide | MS-ESI: 390 (M + 1) |
| 11 | 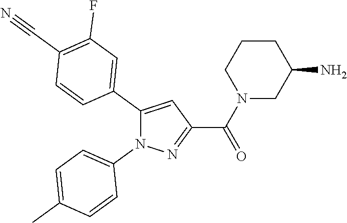 [SMILES: Cc1ccc(-n2nc(C(=O)N3CCC[C@@H](N)C3)cc2-c2ccc(C#N)c(F)c2)cc1] | (R)-4-(3-(3- Aminopiperidine-1- carbonyl)-1-(p-tolyl)-1H- pyrazol-5-yl)-2- fluorobenzonitrile | MS-ESI: 404 (M + 1) |
| 12 | 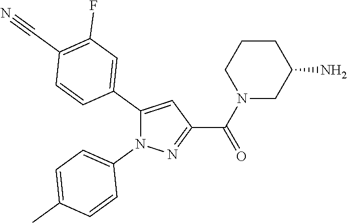 [SMILES: Cc1ccc(-n2nc(C(=O)N3CCC[C@H](N)C3)cc2-c2ccc(C#N)c(F)c2)cc1] | (S)-4-(3-(3- Aminopiperidine-1- carbonyl)-1-(p-tolyl)-1H- pyrazol-5-yl)-2- fluorobenzonitrile | MS-ESI: 404 (M + 1) |
| 13 | 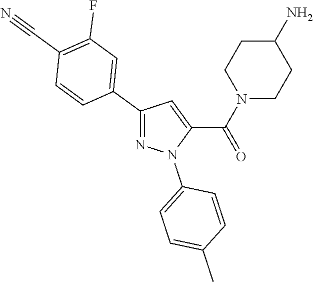 [SMILES: Cc1ccc(-n2nc(-c3ccc(C#N)c(F)c3)cc2C(=O)N2CCC(N)CC2)cc1] | 4-(5-(4-Aminopiperidine- 1-carbonyl)-1-(p-tolyl)- 1H-pyrazol-3-yl)-2- fluorobenzonitrile | MS-ESI: 404 (M + 1) |
| 14 | 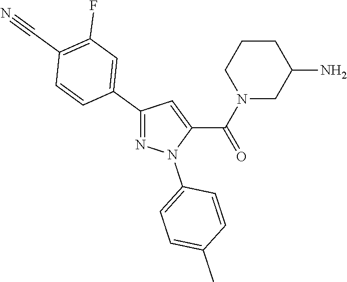 [SMILES: Cc1ccc(-n2nc(-c3ccc(C#N)c(F)c3)cc2C(=O)N2CCCC(N)C2)cc1] | 4-(5-(3-Aminopiperidine- 1-carbonyl)-1-(p-tolyl)- 1H-pyrazol-3-yl)-2- fluorobenzonitrile | MS-ESI: 404 (M + 1) |
| 15 | 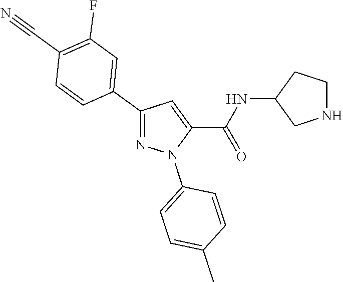 [SMILES: Cc1ccc(-n2nc(-c3ccc(C#N)c(F)c3)cc2C(=O)NC2CCNC2)cc1] | 3-(4-Cyano-3- fluorophenyl)-N- (pyrrolidin-3-yl)-1-(p- tolyl)-1H-pyrazole-5- carboxamide | MS-ESI: 390 (M + 1) |
| 16 | 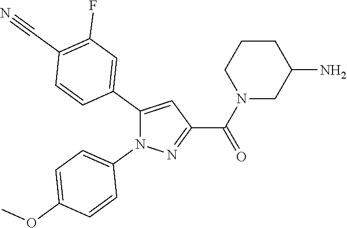 [SMILES: COc1ccc(-n2nc(C(=O)N3CCCC(N)C3)cc2-c2ccc(C#N)c(F)c2)cc1] | 4-(3-(3-Aminopiperidine- 1-carbonyl)-1-(4- methoxyphenyl)-1H- pyrazol-5-yl)-2- fluorobenzonitrile | MS-ESI: 420 (M + 1) |
| 17 | 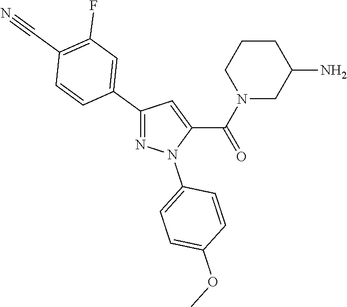 [SMILES: COc1ccc(-n2nc(-c3ccc(C#N)c(F)c3)cc2C(=O)N2CCCC(N)C2)cc1] | 4-(5-(3-Aminopiperidine- 1-carbonyl)-1-(4- methoxyphenyl)-1H- pyrazol-3-yl)-2- fluorobenzonitrile | MS-ESI: 420 (M + 1) |
| 18 | 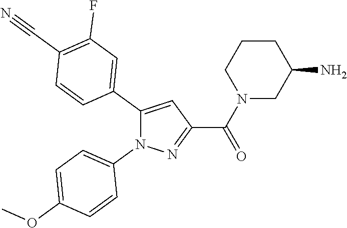 [SMILES: COc1ccc(-n2nc(C(=O)N3CCC[C@@H](N)C3)cc2-c2ccc(C#N)c(F)c2)cc1] | (R)-4-(3-(3- Aminopiperidine-1- carbonyl)-1-(4- methoxyphenyl)-1H- pyrazol-5-yl)-2- fluorobenzonitrile | MS-ESI: 420 (M + 1) |
| 19 | 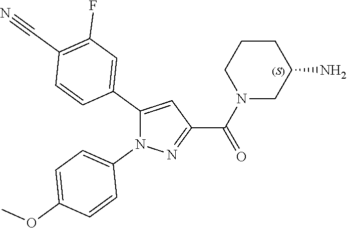 | (S)-4-(3-(3- Aminopiperidine-1- carbonyl)-1-(4- methoxyphenyl)-1H- pyrazol-5-yl)-2- fluorobenzonitrile | MS-ESI: 420 (M + 1) |
| 20 | 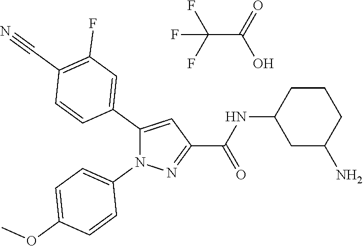 | N-(3-aminocyclohexyl)- 5-(4-cyano-3- fluorophenyl)-1-(4- methoxyphenyl)-1H- pyrazole-3-carboxamide 2,2,2-trifluoroacetate | MS-ESI: 434 (M + 1) |
| 21 | 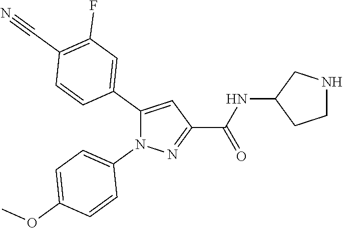 [SMILES: COc1ccc(-n2nc(C(=O)NC3CCNC3)cc2-c2ccc(C#N)c(F)c2)cc1] | 5-(4-Cyano-3- fluorophenyl)-1-(4- methoxyphenyl)-N- (pyrrolidin-3-yl)-1H- pyrazole-3-carboxamide | MS-ESI: 406 (M + 1) |
| 22 | 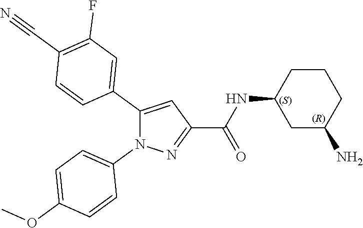 | N-((1S,3R)-3- aminocyclohexyl)-5-(4- cyano-3-fluorophenyl)-1- (4-methoxyphenyl)-1H- pyrazole-3-carboxamide | MS-ESI: 434 (M + 1) |
| 23 | 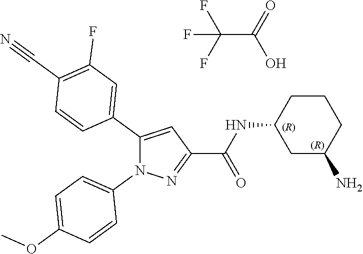 | N-((1R,3R)-3- aminocyclohexyl)-5-(4- cyano-3-fluorophenyl)-1- (4-methoxyphenyl)-1H- pyrazole-3-carboxamide 2,2,2-trifluoroacetate | MS-ESI: 434 (M + 1) |
| 24 | 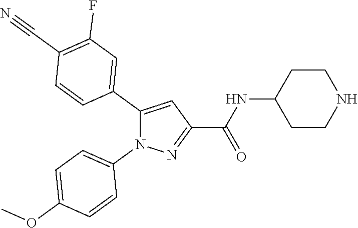 [SMILES: COc1ccc(-n2nc(C(=O)NC3CCNCC3)cc2-c2ccc(C#N)c(F)c2)cc1] | 5-(4-Cyano-3- fluorophenyl)-1-(4- methoxyphenyl)-N- (piperidin-4-yl)-1H- pyrazole-3-carboxamide | MS-ESI: 420 (M + 1) |
| 25 | 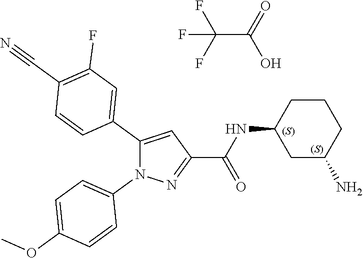 | N-((1S,3S)-3- aminocyclohexyl)-5-(4- cyano-3-fluorophenyl)-1- (4-methoxyphenyl)-1H- pyrazole-3-carboxamide 2,2,2-trifluoroacetate | MS-ESI: 434 (M + 1) |
| 26 | 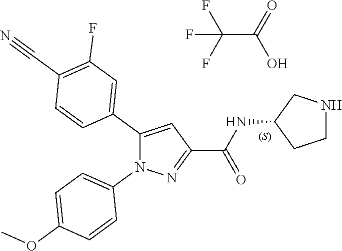 | (S)-5-(4-cyano-3- fluorophenyl)-1-(4- methoxyphenyl)-N- (pyrrolidin-3-yl)-1H- pyrazole-3-carboxamide 2,2,2-trifluoroacetate | MS-ESI: 406 (M + 1) |
| 27 | 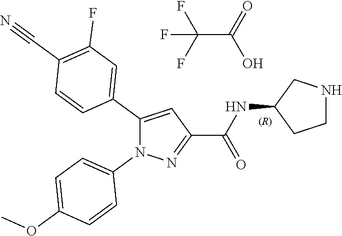 | (R)-5-(4-cyano-3- fluorophenyl)-1-(4- methoxyphenyl)-N- (pyrrolidin-3-yl)-1H- pyrazole-3-carboxamide 2,2,2-trifluoroacetate | MS-ESI: 406 (M + 1) |
| 28 | 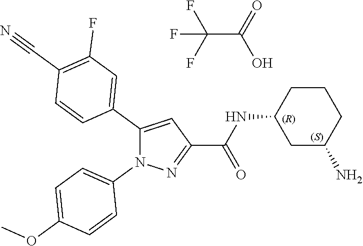 | N-((1R,3S)-3- aminocyclohexyl)-5-(4- cyano-3-fluorophenyl)-1- (4-methoxyphenyl)-1H- pyrazole-3-carboxamide 2,2,2-trifluoroacetate | MS-ESI: 434 (M + 1) |
| 29 | 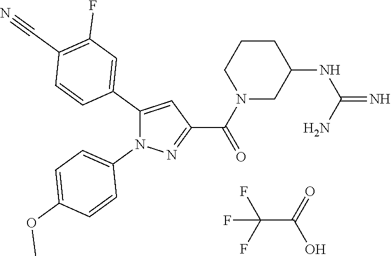 | 1-(1-(5-(4-Cyano-3- fluorophenyl)-1-(4- methoxyphenyl)-1H- pyrazole-3- carbonyl)piperidin-3- yl)guanidine 2,2,2- trifluoroacetate | MS-ESI: 462 (M + 1) |
| 30 | 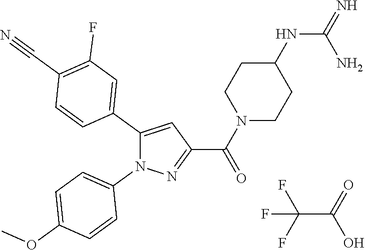 | 1-(1-(5-(4-Cyano-3- fluorophenyl)-1-(4- methoxyphenyl)-1H- pyrazole-3- carbonyl)piperidin-4- yl)guanidine 2,2,2- trifluoroacetate | MS-ESI: 462 (M + 1) |
| 31 | 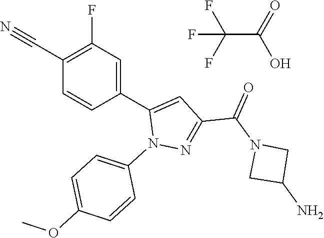 | 4-(3-(3-Aminoazetidine- 1-carbonyl)-1-(4- methoxyphenyl)-1H- pyrazol-5-yl)-2- fluorobenzonitrile 2,2,2- trifluoroacetate | MS-ESI: 392 (M + 1) |
| 32 | 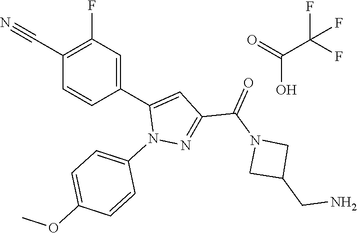 | 4-(3-(3- (Aminomethyl)azetidine- 1-carbonyl)-1-(4- methoxyphenyl)-1H- pyrazol-5-yl)-2- fluorobenzonitrile 2,2,2- trifluoroacetate | MS-ESI: 406 (M + 1) |
| 33 | 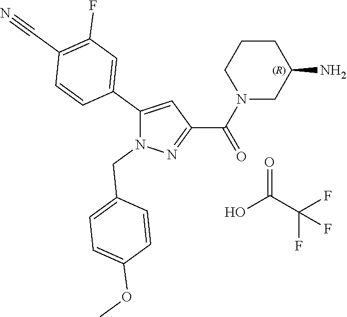 | (R)-4-(3-(3- aminopiperidine-1- carbonyl)-1-(4- methoxybenzyl)-1H- pyrazol-5-yl)-2- fluorobenzonitrile 2,2,2- trifluoroacetate | MS-ESI: 434 (M + 1) |
| 34 | 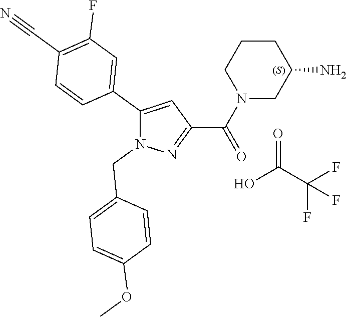 | (S)-4-(3-(3- aminopiperidine-1- carbonyl)-1-(4- methoxybenzyl)-1H- pyrazol-5-yl)-2- fluorobenzonitrile 2,2,2- trifluoroacetate | MS-ESI: 434 (M + 1) |
| 35 | 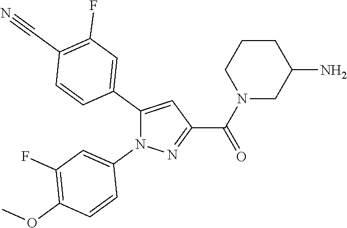 [SMILES: COc1ccc(-n2nc(C(=O)N3CCCC(N)C3)cc2-c2ccc(C#N)c(F)c2)cc1F] | 4-(3-(3-Aminopiperidine- 1-carbonyl)-1-(3-fluoro- 4-methoxyphenyl)-1H- pyrazol-5-yl)-2- fluorobenzonitrile | MS-ESI: 438 (M + 1) |
| 36 | 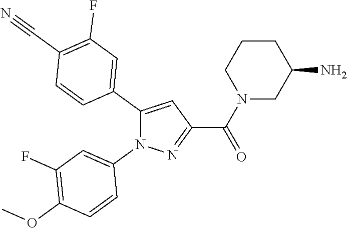 [SMILES: COc1ccc(-n2nc(C(=O)N3CCC[C@@H](N)C3)cc2-c2ccc(C#N)c(F)c2)cc1F] | (R)-4-(3-(3- aminopiperidine-1- carbonyl)-1-(3-fluoro-4- methoxyphenyl)-1H- pyrazol-5-yl)-2- fluorobenzonitrile | MS-ESI: 438 (M + 1) |
| 37 | 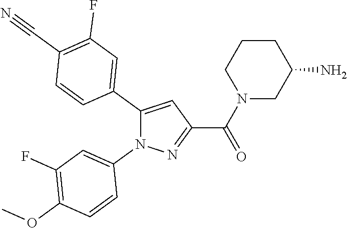 [SMILES: COc1ccc(-n2nc(C(=O)N3CCC[C@H](N)C3)cc2-c2ccc(C#N)c(F)c2)cc1F] | (S)-4-(3-(3- aminopiperidine-1- carbonyl)-1-(3-fluoro-4- methoxyphenyl)-1H- pyrazol-5-yl)-2- fluorobenzonitrile | MS-ESI: 438 (M + 1) |
| 38 | 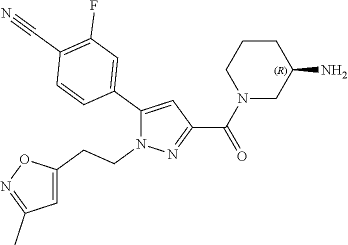 | (R)-4-(3-(3- aminopiperidine-1- carbonyl)-1-(2-(3- methylisoxazol-5- yl)ethyl)-1H-pyrazol-5- yl)-2-fluorobenzonitrile | MS-ESI: 423 (M + 1) |
| 39 | 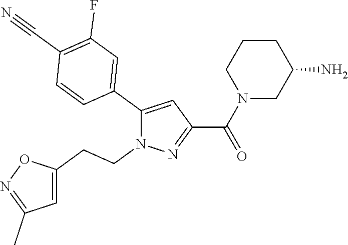 [SMILES: Cc1cc(CCn2nc(C(=O)N3CCC[C@H](N)C3)cc2-c2ccc(C#N)c(F)c2)on1] | (S)-4-(3-(3- aminopiperidine-1- carbonyl)-1-(2-(3- methylisoxazol-5- yl)ethyl)-1H-pyrazol-5- yl)-2-fluorobenzonitrile | MS-ESI: 423 (M + 1) |
| 40 | 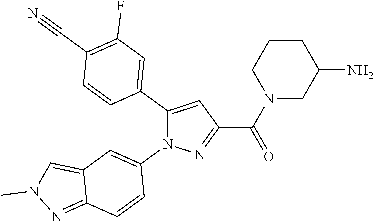 [SMILES: Cn1cc2cc(-n3nc(C(=O)N4CCCC(N)C4)cc3-c3ccc(C#N)c(F)c3)ccc2n1] | 4-(3-(3-Aminopiperidine- 1-carbonyl)-1-(2-methyl- 2H-indazol-5-yl)-1H- pyrazol-5-yl)-2- fluorobenzonitrile | MS-ESI: 444 (M + 1) |
| 41 | 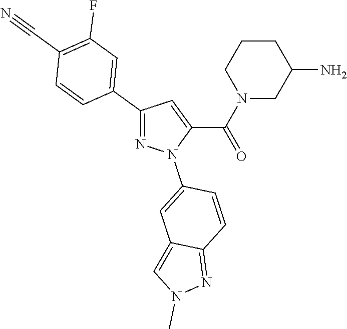 [SMILES: Cn1cc2cc(-n3nc(-c4ccc(C#N)c(F)c4)cc3C(=O)N3CCCC(N)C3)ccc2n1] | 4-(5-(3-Aminopiperidine- 1-carbonyl)-1-(2-methyl- 2H-indazol-5-yl)-1H- pyrazol-3-yl)-2- fluorobenzonitrile | MS-ESI: 444 (M + 1) |
| 42 | 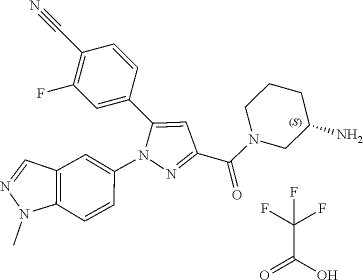 | (S)-4-(3-(3- aminopiperidine-1- carbonyl)-1-(1-methyl- 1H-indazol-5-yl)-1H- pyrazol-5-yl)-2- fluorobenzonitrile 2,2,2- trifluoroacetate | MS-ESI: 444 (M + 1) |
| 43 | 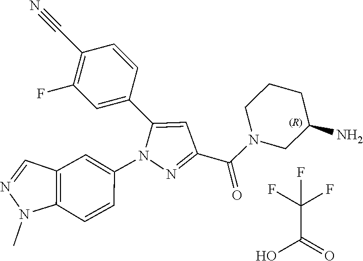 | (R)-4-(3-(3- aminopiperidine-1- carbonyl)-1-(1-methyl- 1H-indazol-5-yl)-1H- pyrazol-5-yl)-2- fluorobenzonitrile 2,2,2- trifluoroacetate | MS-ESI: 444 (M + 1) |
| 44 | 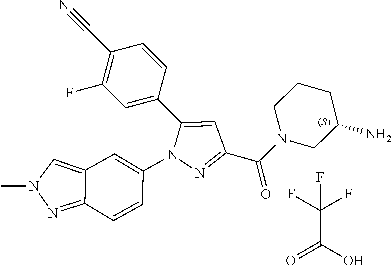 | (S)-4-(3-(3- aminopiperidine-1- carbonyl)-1-(2-methyl- 2H-indazol-5-yl)-1H- pyrazol-5-yl)-2- fluorobenzonitrile 2,2,2- trifluoroacetate | MS-ESI: 444 (M + 1) |

### Example 45

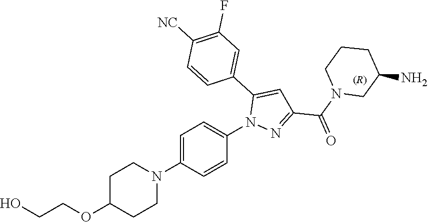

### (R)-4-(3-(3-aminopiperidine-1-carbonyl)-1-(4-(4-(2-hydroxyethoxy)piperidin-1-yl)phenyl)-1H-pyrazol-5-yl)-2-fluorobenzonitrile

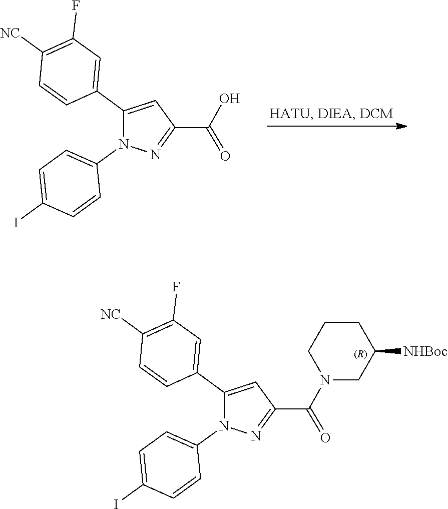

Tert-butyl (R)-(1-(5-(4-cyano-3-fluorophenyl)-1-(4-iodophenyl)-1H-pyrazole-3-carbonyl)piperidin-3-yl)carbamate. To a stirred solution of Intermediate A-13 (1.0 g, 2.3 mmol, 1.0 equiv) in DCM (15 mL) were added tert-butyl (R)-piperidin-3-ylcarbamate (0.46 g, 2.3 mmol, 1.0 equiv), HATU (1.76 g, 4.6 mmol, 2.0 equiv), and DIEA (0.90 g, 6.9 mmol, 3.0 equiv) in portions at rt. The reaction mixture was stirred for 2 h at rt. The reaction mixture was concentrated under vacuum. The residue was eluted from silica gel with DCM/MeOH (10:1). This resulted in 1.32 g (92%) of the title compound as a yellow oil. MS-ESI: 616 (M+1).

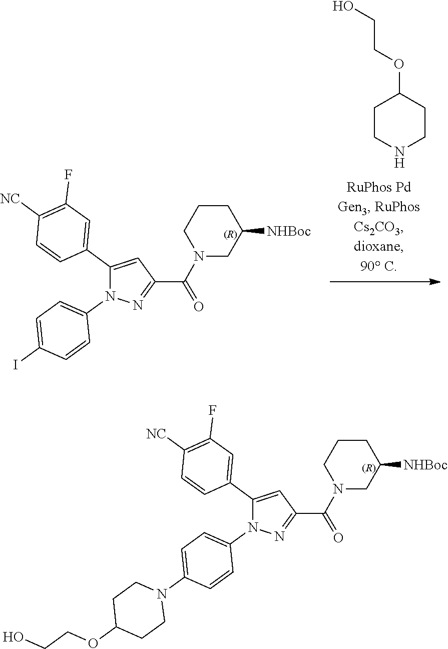

Tert-butyl (R)-(1-(5-(4-cyano-3-fluorophenyl)-1-(4-(4-(2-hydroxyethoxy)piperidin-1-yl)phenyl)-1H-pyrazole-3-carbonyl)piperidin-3-yl)carbamate. To a stirred solution of tert-butyl (R)-(1-(5-(4-cyano-3-fluorophenyl)-1-(4-(4-(2-hydroxyethoxy)piperidin-1-yl)phenyl)-1H-pyrazole-3-carbonyl)piperidin-3-yl)carbamate (500 mg, 0.8 mmol, 1.0 equiv) in dioxane (5 mL) were added 2-(piperidin-4-yloxy)ethan-1-ol (118 mg, 0.8 mmol, 1.0 equiv), RuPhos (2-dicyclohexylphosphino-2′,6′-diisopropoxybiphenyl ligand, available from Sigma-Aldrich; 37.9 mg, 0.08 mmol, 0.1 equiv), Cs₂CO₃ (529 mg, 1.6 mmol, 2.0 equiv) and RuPhos Palladacycle Gen.3 ((2-dicyclohexylphosphino-2′,6′-diisopropoxy-1,1′-biphenyl)[2-(2′-amino-1,1′-biphenyl)]palladium(II) methanesulfonate, available from Sigma-Aldrich; 67.9 mg, 0.08 mmol, 0.1 equiv). The resulting mixture was stirred overnight at 90° C. under a nitrogen atmosphere. The resulting mixture was quenched with water (20 mL) and extracted with ethyl acetate (3×20 mL). The combined organic layers were concentrated under vacuum. The residue was eluted from silica gel with ethyl acetate/petroleum ether (2:1). The collected fractions were combined and concentrated under vacuum. This resulted in 252 mg (49%) of the title compound as a white solid. MS-ESI: 633 (M+1).

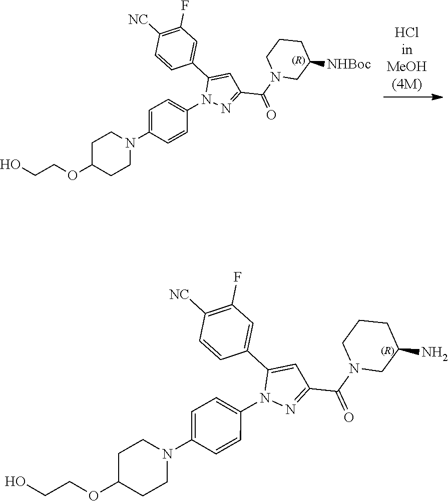

(R)-4-(3-(3-aminopiperidine-1-carbonyl)-1-(4-(4-(2-hydroxyethoxy)piperidin-1-yl)phenyl)-1H-pyrazol-5-yl)-2-fluorobenzonitrile. To a stirred solution of tert-butyl (R)-(1-(5-(4-cyano-3-fluorophenyl)-1-(4-(4-(2-hydroxyethoxy)piperidin-1-yl)phenyl)-1H-pyrazole-3-carbonyl)piperidin-3-yl)carbamate (240 mg, 0.4 mmol, 1.0 equiv) in HCl (g) in MeOH (4M, 5 mL). The resulting mixture was stirred for 1 h at rt. The mixture was basified to pH 7 with saturated Na₂CO₃ (aq.). The resulting mixture was extracted with ethyl acetate (3×20 mL). The combined organic layers were concentrated under vacuum. The crude product (200 mg) was purified by Prep-HPLC with the following conditions: Column: XBridge Shield RP18 OBD Column, 30*150 mm, 5 μm; Mobile Phase A: water (10 mM NH₄HCO₃+0.1% NH₃·H₂O), Mobile Phase B: ACN; Flow rate: 60 mL/min; Gradient: 18% B to 43% B in 8 min; Detector UV: 220 nm; Rₜ 1 (min): 7.62. This resulted in 74 mg (36%) of the title compound as a yellow solid. MS-ESI: 533 (M+1). ¹H NMR (400 MHz, MeOD-d₄) δ 7.73 (t, J=7.4 Hz, 1H), 7.35-7.25 (m, 2H), 7.24-7.15 (m, 2H), 7.06-6.99 (m, 3H), 4.55-4.32 (m, 2H), 3.73-3.54 (m, 7H), 3.31-3.25 (m, 1H), 3.19-3.00 (m, 3H), 2.91-2.77 (m, 1H), 2.10-1.99 (m, 3H), 1.93-1.77 (m, 1H), 1.76-1.54 (m, 3H), 1.52-1.35 (m, 1H).

Examples in the following table were prepared using similar conditions as described for those of Example 45 from the appropriate starting materials.

TABLE 5
Example Compounds.

| Ex. | | | |
|---|---|---|---|
| No. | Structure | Name | Data |
| 46 | 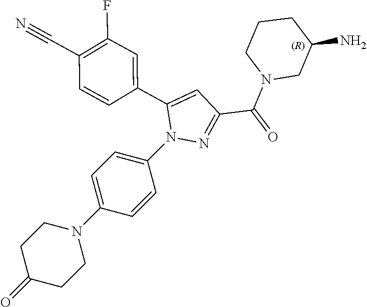 | (R)-4-(3-(3- aminopiperidine-1- carbonyl)-1-(4-(4- oxopiperidin-1- yl)phenyl)-1H-pyrazol-5- yl)-2-fluorobenzonitrile | MS-ESI: 487(M + 1) |
| 47 | 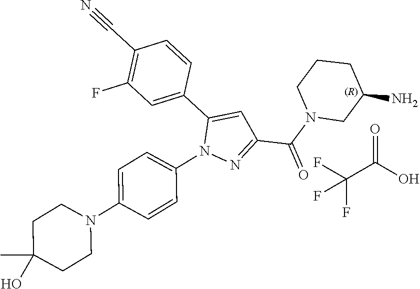 | (R)-4-(3-(3- aminopiperidine-1- carbonyl)-1-(4-(4- hydroxy-4- methylpiperidin-1- yl)phenyl)-1H-pyrazol-5- yl)-2-fluorobenzonitrile 2,2,2-trifluoroacetate | MS-ESI: 503(M + 1) |
| 48 | 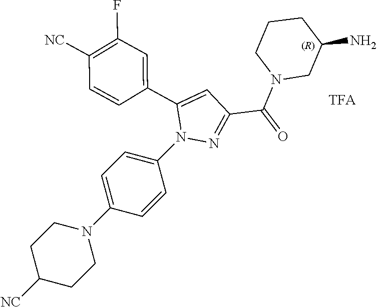 | (R)-1-(4-(3-(3- aminopiperidine-1- carbonyl)-5-(4-cyano-3- fluorophenyl)-1H- pyrazol-1- yl)phenyl)piperidine-4- carbonitrile 2,2,2- trifluoroacetate | MS-ESI: 498(M + 1) |
| 49 | 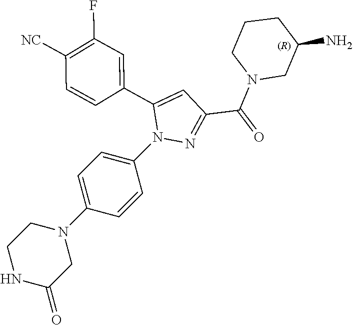 | (R)-4-(3-(3- aminopiperidine-1- carbonyl)-1-(4-(3- oxopiperazin-1- yl)phenyl)-1H-pyrazol-5- yl)-2-fluorobenzonitrile | MS-ESI: 488(M + 1) |
| 50 | 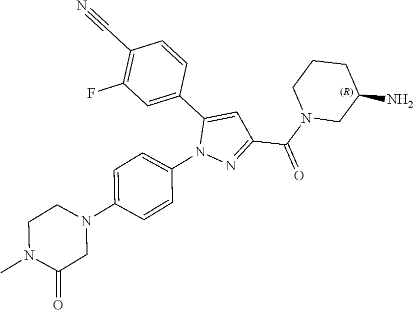 | (R)-4-(3-(3- aminopiperidine-1- carbonyl)-1-(4-(4-methyl- 3-oxopiperazin-1- yl)phenyl)-1H-pyrazol-5- yl)-2-fluorobenzonitrile | MS-ESI: 502(M + 1) |
| 51 | 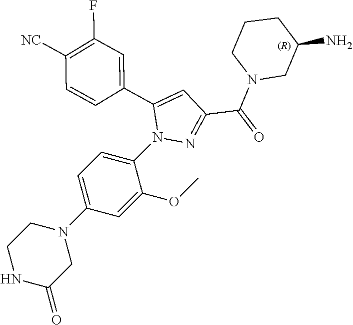 | (R)-4-(3-(3- aminopiperidine-1- carbonyl)-1-(2-methoxy- 4-(3-oxopiperazin-1- yl)phenyl)-1H-pyrazol-5- yl)-2-fluorobenzonitrile | MS-ESI: 518(M + 1) |
| 53 | 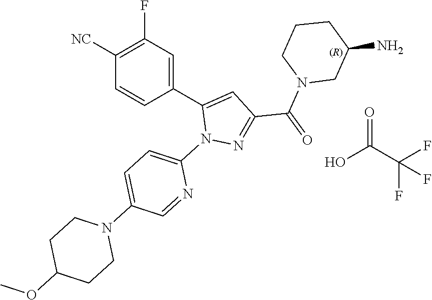 | (R)-4-(3-(3- aminopiperidine-1- carbonyl)-1-(5-(4- methoxypiperidin-1- yl)pyridin-2-yl)-1H- pyrazol-5-yl)-2- fluorobenzonitrile 2,2,2- trifluoroacetate | MS-ESI: 504(M + 1) |
| 54 | 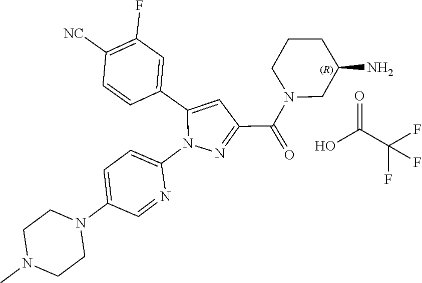 | (R)-4-(3-(3- aminopiperidine-1- carbonyl)-1-(5-(4- methylpiperazin-1- yl)pyridin-2-yl)-1H- pyrazol-5-yl)-2- fluorobenzonitrile 2,2,2- trifluoroacetate | MS-ESI: 489(M + 1) |
| 55 | 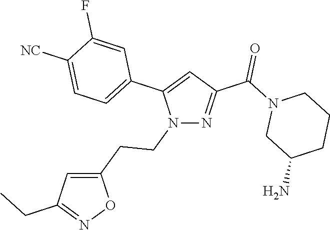 [SMILES: CCc1cc(CCn2nc(C(=O)N3CCC[C@H](N)C3)cc2-c2ccc(C#N)c(F)c2)on1] | (S)-4-(3-(3-amino- piperidine-1-carbonyl)-1- (2-(3-ethylisoxazol-5- yl)ethyl)-1H-pyrazol-5-yl)- 2-fluorobenzonitrile | |
| 56 | 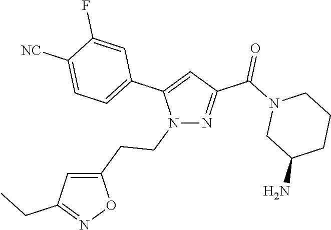 [SMILES: CCc1cc(CCn2nc(C(=O)N3CCC[C@@H](N)C3)cc2-c2ccc(C#N)c(F)c2)on1] | (R)-4-(3-(3-amino- piperidine-1-carbonyl)-1- (2-(3-ethylisoxazol-5- yl)ethyl)-1H-pyrazol-5-yl)- 2-fluorobenzonitrile | |
| 57 | 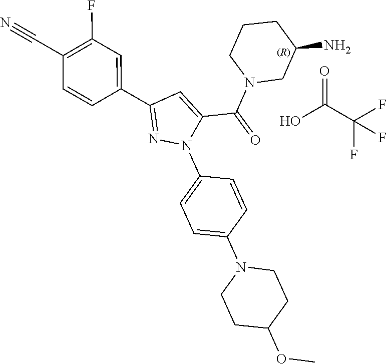 | (R)-4-(5-(3- aminopiperidine-1- carbonyl)-1-(4-(4- methoxypiperidin-1- yl)phenyl)-1H-pyrazol-3- yl)-2-fluorobenzonitrile 2,2,2-trifluoroacetate | MS-ESI: 503(M + 1) |
| 58 | 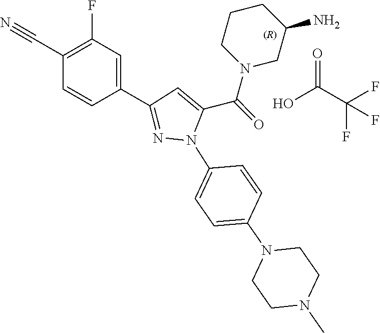 | (R)-4-(5-(3- aminopiperidine-1- carbonyl)-1-(4-(4- methylpiperazin-1- yl)phenyl)-1H-pyrazol-3- yl)-2-fluorobenzonitrile 2,2,2-trifluoroacetate | MS-ESI: 488(M + 1) |
| 59 | 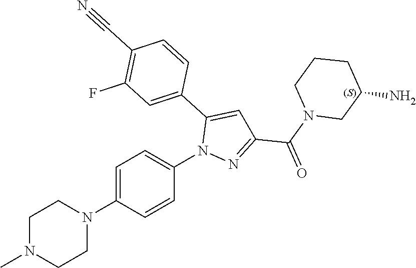 | (S)-4-(3-(3- aminopiperidine-1- carbonyl)-1-(4-(4- methylpiperazin-1- yl)phenyl)-1H-pyrazol-5- yl)-2-fluorobenzonitrile | MS-ESI: 488(M + 1) |
| 60 | 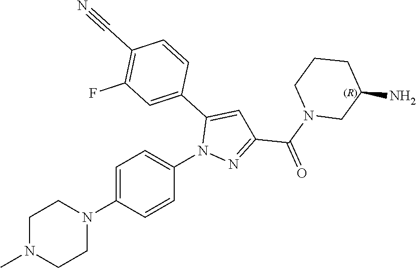 | (R)-4-(3-(3- aminopiperidine-1- carbonyl)-1-(4-(4- methylpiperazin-1- yl)phenyl)-1H-pyrazol-5- yl)-2-fluorobenzonitrile | MS-ESI: 488(M + 1) |
| 61 | 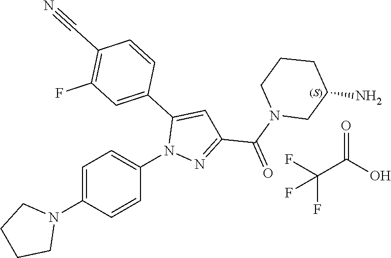 | (S)-4-(3-(3- aminopiperidine-1- carbonyl)-1-(4- (pyrrolidin-1-yl)phenyl)- 1H-pyrazol-5-yl)-2- fluorobenzonitrile 2,2,2- trifluoroacetate | MS-ESI: 459(M + 1) |
| 62 | 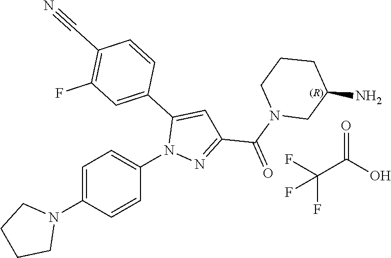 | (R)-4-(3-(3- aminopiperidine-1- carbonyl)-1-(4- (pyrrolidin-1-yl)phenyl)- 1H-pyrazol-5-yl)-2- fluorobenzonitrile 2,2,2- trifluoroacetate | MS-ESI: 459(M + 1) |
| 63 | 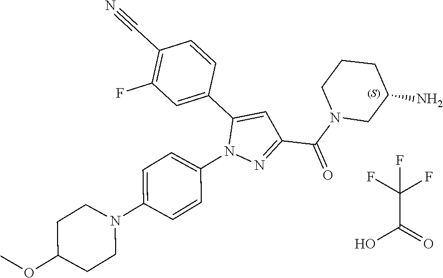 | (S)-4-(3-(3- aminopiperidine-1- carbonyl)-1-(4-(4- methoxypiperidin-1- yl)phenyl)-1H-pyrazol-5- yl)-2-fluorobenzonitrile 2,2,2-trifluoroacetate | MS-ESI: 503(M + 1) |
| 64 | 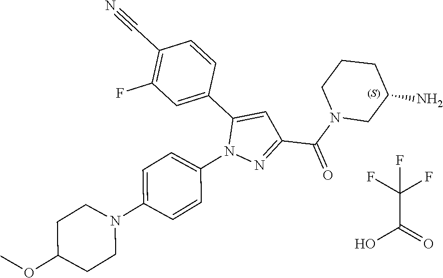 | (R)-4-(3-(3- aminopiperidine-1- carbonyl)-1-(4-(4- methoxypiperidin-1- yl)phenyl)-1H-pyrazol-5- yl)-2-fluorobenzonitrile 2,2,2-trifluoroacetate | MS-ESI: 503(M + 1) |
| 65 | 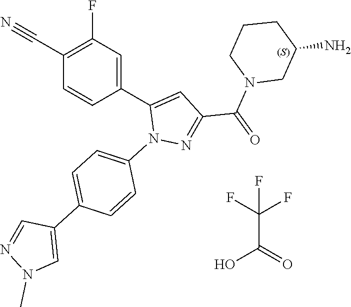 | (S)-4-(3-(3- aminopiperidine-1- carbonyl)-1-(4-(1-methyl- 1H-pyrazol-4-yl)phenyl)- 1H-pyrazol-5-yl)-2- fluorobenzonitrile 2,2,2- trifluoroacetate | MS-ESI: 470(M + 1) |
| 66 | 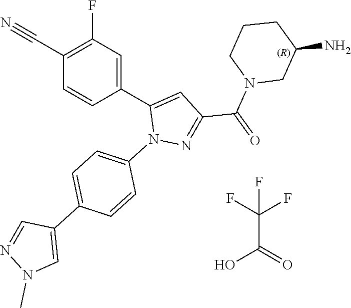 | (R)-4-(3-(3- aminopiperidine-1- carbonyl)-1-(4-(1-methyl- 1H-pyrazol-4-yl)phenyl)- 1H-pyrazol-5-yl)-2- fluorobenzonitrile 2,2,2- trifluoroacetate | MS-ESI: 470(M + 1) |
| 67 | 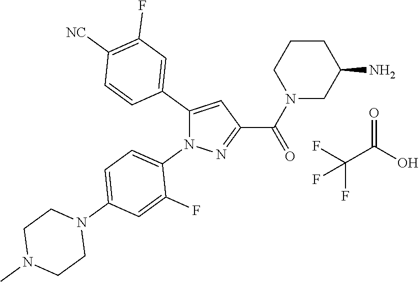 | (R)-4-(3-(3- aminopiperidine-1- carbonyl)-1-(2-fluoro-4- (4-methylpiperazin-1- yl)phenyl)-1H-pyrazol-5- yl)-2-fluorobenzonitrile | MS-ESI: 506(M + 1) |
| 68 | 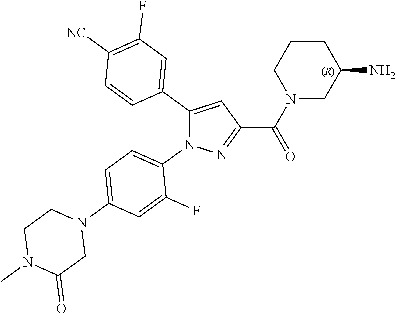 | (R)-4-(3-(3- aminopiperidine-1- carbonyl)-1-(2-fluoro-4- (4-methyl-3- oxopiperazin-1- yl)phenyl)-1H-pyrazol-5- yl)-2-fluorobenzonitrile | MS-ESI: 520(M + 1) |

### Example 69

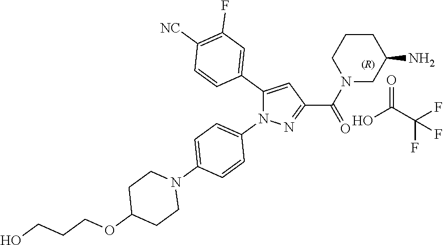

### (R)-4-(3-(3-Aminopiperidine-1-carbonyl)-1-(4-(4-(3-hydroxypropoxy)piperidin-1-yl)phenyl)-1H-pyrazol-5-yl)-2-fluorobenzonitrile 2,2,2-trifluoroacetate

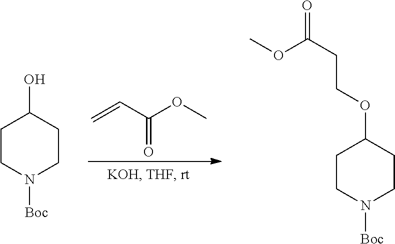

Tert-butyl 4-(3-methoxy-3-oxopropoxy)piperidine-1-carboxylate. To a stirred solution of tert-butyl 4-hydroxypiperidine-1-carboxylate (8.84 g, 43.9 mmol, 1.0 equiv) in THF (50 mL) were added methyl acrylate (7.57 g, 87.9 mmol, 2.0 equiv) in THF (50 mL) was added KOH (0.25 g, 4.4 mmol, 0.1 equiv) in portions at rt under a nitrogen atmosphere. The resulting mixture was stirred overnight at rt under a nitrogen atmosphere. The residue was purified by silica gel with petroleum ether/ethyl acetate (5:1) to afford the title compound (1.8 g, 14.2%) as a yellow oil. MS-ESI: 288 (M+1).

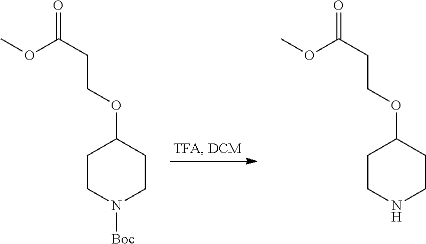 [SMILES: COC(=O)CCOC1CCN(C(=O)OC(C)(C)C)CC1 ; COC(=O)CCOC1CCNCC1]

Methyl 3-(piperidin-4-yloxy)propanoate. To a stirred solution of tert-butyl 4-(3-methoxy-3-oxopropoxy)piperidine-1-carboxylate (1.8 g, 6.3 mmol, 1 equiv) in DCM (20 mL) was added TFA (4 mL). The reaction solution was stirred for 1 h at rt. The resulting mixture was concentrated under vacuum. This resulted in 3.2 g (crude) of the title compound as a yellow oil. MS-ESI: 188 (M+1).

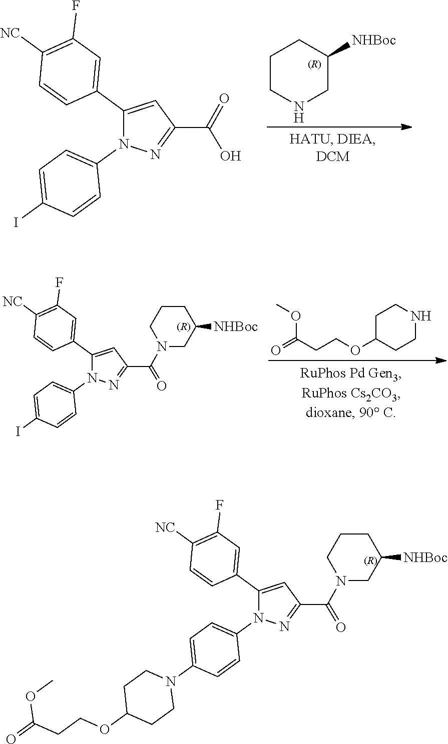

Methyl (R)-3-((1-(4-(3-(3-((tert-butoxycarbonyl)amino)piperidine-1-carbonyl)-5-(4-cyano-3-fluorophenyl)-1H-pyrazol-1-yl)phenyl)piperidin-4-yl)oxy)propanoate. Intermediate A-13 was converted to the title compound using similar procedures as those used in Example 45. MS-ESI: 675 (M+1).

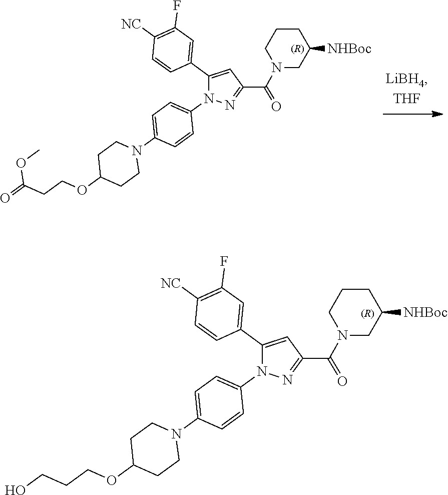

Tert-butyl (R)-(1-(5-(4-cyano-3-fluorophenyl)-1-(4-(4-(3-hydroxypropoxy) piperidin-1-yl)phenyl)-1H-pyrazole-3-carbonyl)piperidin-3-yl)carbamate. To a stirred solution of methyl (R)-3-((1-(4-(3-(3-((tert-butoxycarbonyl)amino)piperidine-1-carbonyl)-5-(4-cyano-3-fluorophenyl)-1H-pyrazol-1-yl)phenyl)piperidin-4-yl)oxy)propanoate (650 mg, 1.0 mmol, 1.0 equiv) in THE (7 mL) was added LiBH₄ (105 mg, 4.8 mmol, 5.0 equiv). The reaction mixture was stirred for 1 h at rt. The reaction mixture was quenched with H₂O (10 mL) and extracted with DCM (3×50 mL). The combined organic layers were washed with sat. NaCl (aq.) (3×50 mL), dried over anhydrous Na₂SO₄, and concentrated under vacuum. The residue was purified by Prep-TLC (ethyl acetate). This resulted in 319 mg (51.2%) of the title compound as a yellow solid. MS-ESI: 647 (M+1).

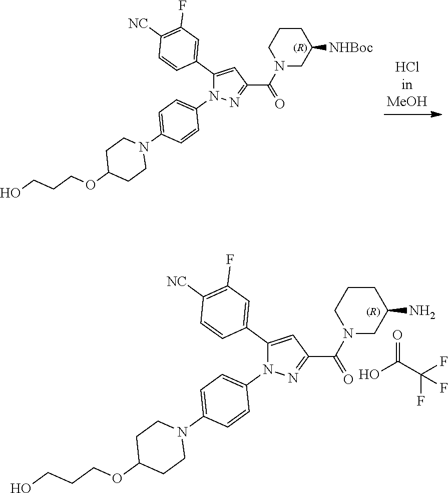

(R)-4-(3-(3-Aminopiperidine-1-carbonyl)-1-(4-(4-(3-hydroxypropoxy)piperidin-1-yl)phenyl)-1H-pyrazol-5-yl)-2-fluorobenzonitrile 2,2,2-trifluoroacetate. A solution of tert-butyl (R)-(1-(5-(4-cyano-3-fluorophenyl)-1-(4-(4-(3-hydroxypropoxy)piperidin-1-yl)phenyl)-1H-pyrazole-3-carbonyl)piperidin-3-yl)carbamate (130 mg, 0.201 mmol, 1.0 equiv) in HCl (g) in MeOH (4 M, 3 mL) was stirred for 1 h at rt. The reaction mixture was concentrated under vacuum. The crude product was purified by Prep-HPLC with the following conditions: Column: YMC-Actus Triart C18 ExRS, 30*150 mm, 5 μm; Mobile Phase A: water (10 mM NH₄HCO₃), Mobile Phase B: MeOH; Flow rate: 60 mL/min; Gradient: 40% B to 68% B in 8 min; Detector UV: 220 nm; Rₜ 1 (min): 7.6. This resulted in 28.2 mg (25.6%) of the title compound as a yellow solid. MS-ESI: 547 (M+1). ¹H NMR (400 MHz, MeOD-d₄) δ 7.72 (t, J=7.4 Hz, 1H), 7.29 (d, J=9.2 Hz, 1H), 7.27 (dd, J=8.0, 1.2 Hz, 1H), 7.20 (t, J=9.2 Hz, 2H), 7.05-6.97 (m, 3H), 4.56-4.25 (m, 2H), 3.75-3.42 (m, 7H), 3.24-3.09 (m, 1H), 3.09-2.77 (m, 4H), 2.09-1.94 (m, 3H), 1.89-1.74 (m, 3H), 1.72-1.52 (m, 3H), 1.54-1.38 (m, 1H).

### Example 70

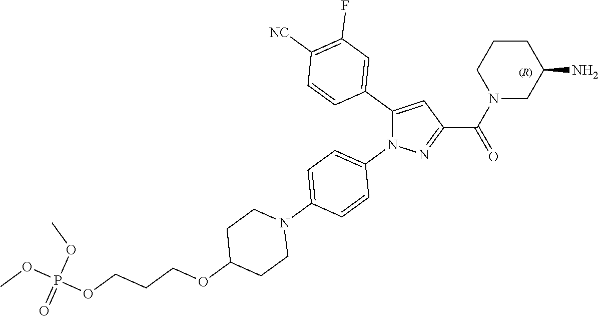

### (R)-3-((1-(4-(3-(3-aminopiperidine-1-carbonyl)-5-(4-cyano-3-fluorophenyl)-1H-pyrazol-1-yl)phenyl)piperidin-4-yl)oxy)propyl dimethyl phosphate 2,2,2-trifluoroacetate

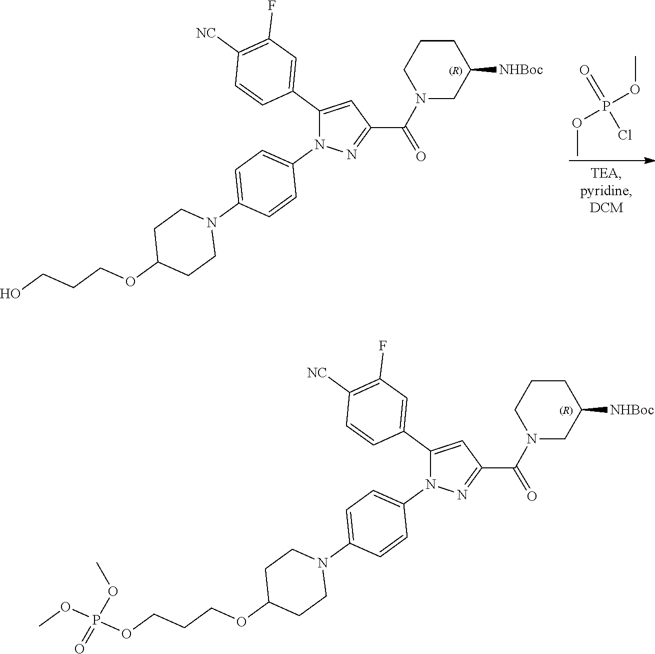

Tert-butyl (R)-(1-(5-(4-cyano-3-fluorophenyl)-1-(4-(4-(3-((dimethoxyphosphoryl)oxy)propoxy)piperidin-1-yl)phenyl)-1H-pyrazole-3-carbonyl)piperidin-3-yl)carbamate. To a stirred solution of tert-butyl (R)-(1-(5-(4-cyano-3-fluorophenyl)-1-(4-(4-(3-hydroxypropoxy)piperidin-1-yl)phenyl)-1H-pyrazole-3-carbonyl)piperidin-3-yl)carbamate (Example 69; 250 mg, 0.39 mmol, 1.0 equiv) in DCM (10 mL) were added Pyridine (92 mg, 1.2 mmol, 3.0 equiv) and TEA (117 mg, 1.2 mmol, 3.0 equiv). To above mixture was added dimethyl phosphorochloridate (168 mg, 1.2 mmol, 3.0 equiv) dropwise at 0° C. under a nitrogen atmosphere. The resulting mixture was stirred for 16 h at room temperature under a nitrogen atmosphere. The resulting mixture was concentrated under vacuum. The residue was purified by Prep-TLC (DCM/MeOH 20:1) to afford the title compound (100 mg, 34.2%) as a yellow solid. MS-ESI: 755 (M+1).

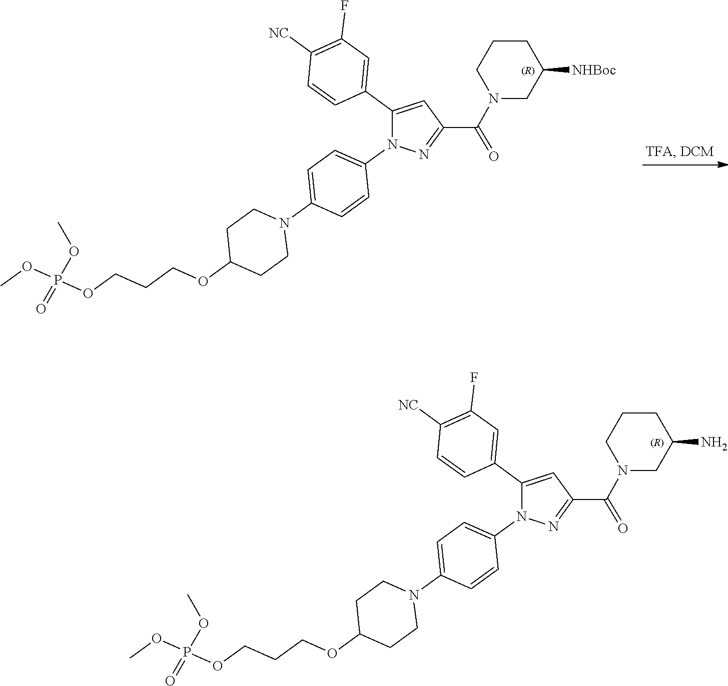

(R)-3-((1-(4-(3-(3-aminopiperidine-1-carbonyl)-5-(4-cyano-3-fluorophenyl)-1H-pyrazol-1-yl)phenyl)piperidin-4-yl)oxy)propyl dimethyl phosphate 2,2,2-trifluoroacetate. A solution of tert-butyl (R)-(1-(5-(4-cyano-3-fluorophenyl)-1-(4-(4-(3-((dimethoxyphosphoryl)oxy) propoxy)piperidin-1-yl)phenyl)-1H-pyrazole-3-carbonyl)piperidin-3-yl)carbamate (40 mg, 0.05 mmol, 1.0 equiv) in DCM (5 mL) and TFA (1 mL) was stirred for overnight at room temperature. The resulting mixture was concentrated under vacuum. The crude product was purified by Prep-HPLC with the following conditions (Column: XBridge Shield RP18 OBD Column, 30*150 mm, 5 μm; Mobile Phase A: Water (0.05% TFA), Mobile Phase B: ACN; Flow rate: 60 mL/min; Gradient: 21% B to 31% B in 8 min; Detector UV: 220; 254 nm; Rₜ 1 (min): 7.50) to afford the title compound (13 mg, 31.91%) as a yellow oil. MS-ESI: 655 (M+1). ¹H NMR (400 MHz, MeOD-d₄) δ 7.75 (t, J=7.4 Hz, 1H), 7.33-7.24 (m, 4H), 7.15-7.10 (m, 3H), 4.73-4.32 (m, 2H), 4.209 (q, J=6.4 Hz, 2H), 3.79 (d, J=11.2 Hz, 6H), 3.68-3.61 (m, 5H), 3.41-3.33 (m, 2H), 3.19-3.13 (m, 2H), 2.23-2.17 (m, 1H), 2.09-1.88 (m, 5H), 1.84-1.75 (m, 5H).

### Example 71

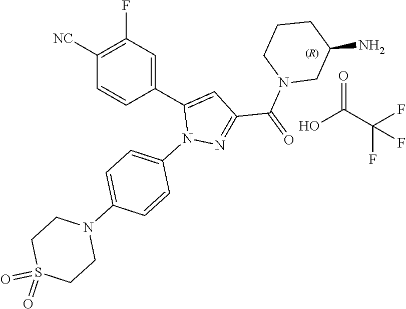

### (R)-4-(3-(3-aminopiperidine-1-carbonyl)-1-(4-(1,1-dioxidothiomorpholino)phenyl)-1H-pyrazol-5-yl)-2-fluorobenzonitrile 2,2,2-trifluoroacetate

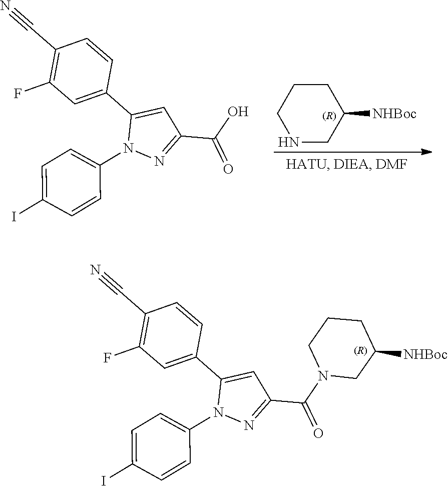

Tert-butyl (R)-(1-(5-(4-cyano-3-fluorophenyl)-1-(4-iodophenyl)-1H-pyrazole-3-carbonyl)piperidin-3-yl)carbamate. To a stirred solution of 5-(4-cyano-3-fluorophenyl)-1-(4-iodophenyl)-1H-pyrazole-3-carboxylic acid (430 mg, 0.99 mmol, 1.0 equiv) in DMF (5 mL) were added tert-butyl (R)-piperidin-3-ylcarbamate (199 mg, 0.99 mmol, 1.0 equiv), DIEA (385 mg, 2.98 mmol, 3.0 equiv) and HATU (755 mg, 1.99 mmol, 2.0 equiv). The reaction mixture was stirred for 2 h at room temperature. The residue was diluted with water (20 mL) and extracted with EtOAc (3×20 mL). The combined organic layers were washed with H₂O (3×20 mL), dried with anhydrous Na₂SO₄ and concentrated under vacuum. The residue was eluted from silica gel with DCM/MeOH (10:1). This resulted in 320 mg (45%) of the title compound as yellow solid. MS-ESI: 616 (M+1).

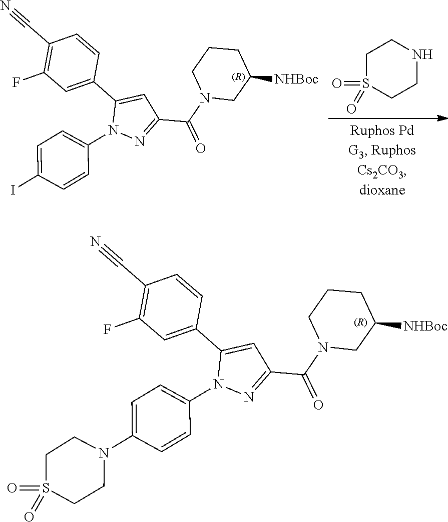

Tert-butyl (R)-(1-(5-(4-cyano-3-fluorophenyl)-1-(4-(1,1-dioxidothiomorpholino) phenyl)-1H-pyrazole-3-carbonyl)piperidin-3-yl)carbamate. To a stirred solution of tert-butyl (R)-(1-(5-(4-cyano-3-fluorophenyl)-1-(4-iodophenyl)-1H-pyrazole-3-carbonyl)piperidin-3-yl)carbamate (300 mg, 0.49 mmol, 1.0 equiv) in dioxane (10 mL) were added thiomorpholine 1,1-dioxide (66 mg, 0.49 mmol, 1.0 equiv), RuPhos (2-dicyclohexylphosphino-2′,6′-diisopropoxybiphenyl ligand, available from Sigma-Aldrich; 22.8 mg, 0.049 mmol, 0.10 equiv), RuPhos Palladacycle Gen.3 (((2-dicyclohexylphosphino-2′,6′-diisopropoxy-1,1′-biphenyl)[2-(2′-amino-1,1′-biphenyl)]palladium(II) methanesulfonate, available from Sigma-Aldrich; 40.8 mg, 0.049 mmol, 0.10 equiv) and Cs₂CO₃ (476 mg, 1.46 mmol, 3.0 equiv) under a nitrogen atmosphere. The reaction mixture was stirred overnight at 90° C. under a nitrogen atmosphere. The residue was diluted with water (30 mL) and extracted with EtOAc (3×30 mL). The combined organic layers were dried over anhydrous Na₂SO₄ and concentrated under vacuum. The residue was eluted from silica gel with EtOAc/PE (5:1). This resulted in 237 mg (68.7%) of the title compound as a yellow solid. MS-ESI: 623 (M+1).

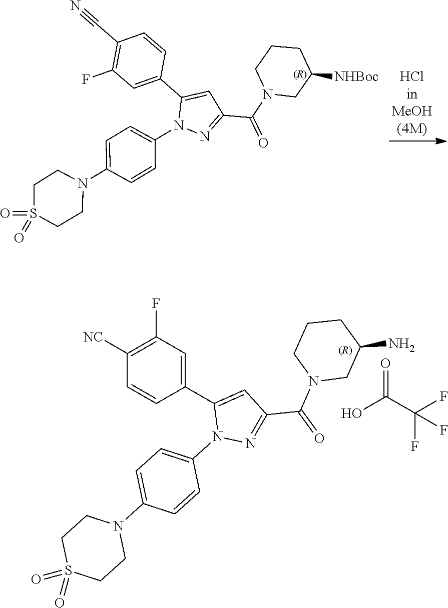

(R)-4-(3-(3-aminopiperidine-1-carbonyl)-1-(4-(1,1-dioxidothiomorpholino) phenyl)-1H-pyrazol-5-yl)-2-fluorobenzonitrile 2,2,2-trifluoroacetate. A solution of tert-butyl (R)-(1-(5-(4-cyano-3-fluorophenyl)-1-(4-(1,1-dioxidothiomorpholino)phenyl)-1H-pyrazole-3-carbonyl)piperidin-3-yl)carbamate (180 mg, 0.29 mmol, 1.0 equiv) in HCl (g) in MeOH (4M, 3 mL) was stirred for 2 h at room temperature. The mixture was adjusted to pH 8 with sat. NaHCO₃ (aq.). The resulting mixture was extracted with EtOAc (3×20 mL). The combined organic layers were dried with anhydrous Na₂SO₄ and concentrated under vacuum. The residue was eluted from silica gel with DCM/MeOH (10:1). The crude product (210 mg) was purified by Prep-HPLC with the following conditions: SunFire Prep C18 OBD Column, 19*150 mm, 5 μm; Mobile Phase A: water (0.05% TFA), Mobile Phase B: ACN; Flow rate: 20 mL/min; Gradient: 40% B to 70% B in 6.2 min, Detector UV: 210/254 nm; Rₜ 1 (min): 5.54. This resulted in 115 mg (60%) of the title compound as a yellow solid. MS-ESI: 523 (M+1). ¹H NMR (400 MHz, MeOD-d₄) δ 7.76 (t, J=7.6 Hz, 1H), 7.39-7.23 (m, 4H), 7.19-7.06 (m, 3H), 4.78-4.07 (m, 2H), 3.96 (t, J=5.4 Hz, 4H), 3.82-3.59 (m, 1H), 3.49-3.37 (m, 2H), 3.17 (t, J=5.4 Hz, 4H), 2.28-2.12 (m, 1H), 2.01-1.86 (m, 1H), 1.85-1.67 (m, 2H).

Examples in the following table were prepared using similar conditions as described for those of Example 71 from the appropriate starting materials.

TABLE 6
Example Compounds.

| Ex. | | | |
|---|---|---|---|
| No. | Structure | Name | Data |
| 80 | 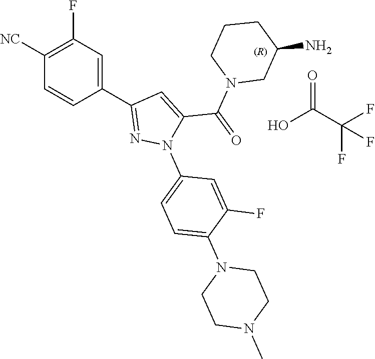 | (R)-4-(5-(3- aminopiperidine-1- carbonyl)-1-(3-fluoro-4- (4-methylpiperazin-1- yl)phenyl)-1H-pyrazol-3- yl)-2-fluorobenzonitrile 2,2,2-trifluoroacetate | MS-ESI: 506(M + 1) |

### Example 84

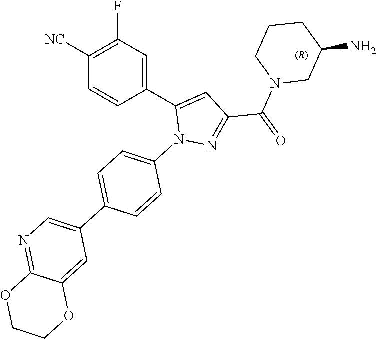

### (R)-4-(3-(3-aminopiperidine-1-carbonyl)-1-(4-(2,3-dihydro-[1,4]dioxino[2,3-b]pyridin-7-yl)phenyl)-1H-pyrazol-5-yl)-2-fluorobenzonitrile 2,2,2-trifluoroacetate

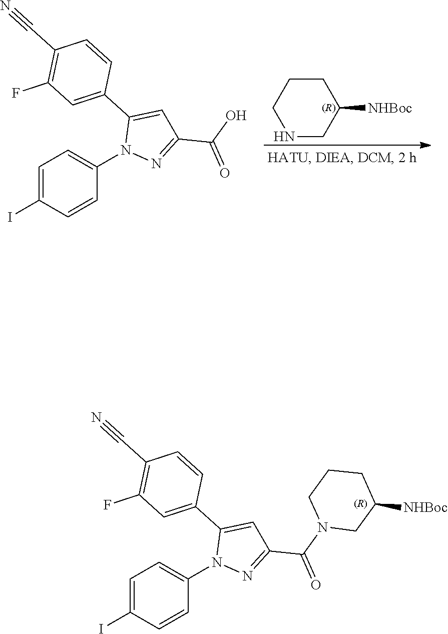

Tert-butyl (R)-(1-(5-(4-cyano-3-fluorophenyl)-1-(4-iodophenyl)-1H-pyrazole-3-carbonyl)piperidin-3-yl)carbamate. To a stirred solution of 5-(4-cyano-3-fluorophenyl)-1-(4-iodophenyl)-1H-pyrazole-3-carboxylic acid (430 mg, 0.99 mmol, 1.0 equiv) in DMF (5 mL) were added tert-butyl (R)-piperidin-3-ylcarbamate (199 mg, 0.99 mmol, 1.0 equiv), DIEA (385 mg, 2.98 mmol, 3.0 equiv), and HATU (755 mg, 1.99 mmol, 2.0 equiv) in portions at room temperature. The reaction mixture was stirred for 2 h at room temperature. The residue was diluted with water (20 mL) and extracted with EtOAc (3×20 mL). The combined organic layers were dried with anhydrous Na₂SO₄ and concentrated under vacuum. The residue was eluted from silica gel with DCM/MeOH (10:1). This resulted in 320 mg (45%) of the title compound as yellow solid. MS-ESI: 616 (M+1).

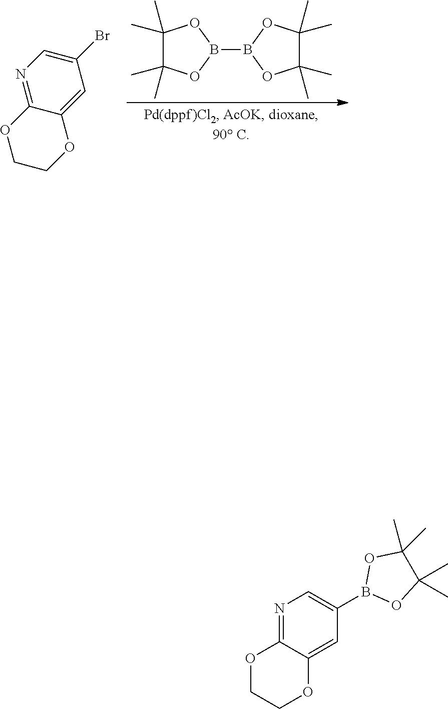

7-(4,4,5,5-Tetramethyl-1,3,2-dioxaborolan-2-yl)-2,3-dihydro-[1,4]dioxino[2,3-b]pyridine. To a stirred solution of 7-bromo-2,3-dihydro-[1,4]dioxino[2,3-b]pyridine (5.0 g, 23.1 mmol, 1.0 equiv) in dioxane (50 mL) were added bis(pinacolato)diboron (5.93 g, 23.4 mmol, 1.0 equiv), Pd(dppf)Cl₂-DCM (1.89 g, 2.32 mmol, 0.1 equiv) and AcOK (5.68 g, 57.9 mmol, 2.5 equiv) in portions at room temperature under a nitrogen atmosphere. The resulting mixture was stirred overnight at 90° C. under a nitrogen atmosphere. The mixture was allowed to cool down to room temperature. The resulting mixture was filtered, and the filter cake was washed with DCM (3×100 mL). The filtrate was concentrated under vacuum. The residue was eluted from silica gel with PE/EtOAc (1:1). This resulted in 5.6 g (91%) of the title compound as a yellow oil. MS-ESI: 264 (M+1).

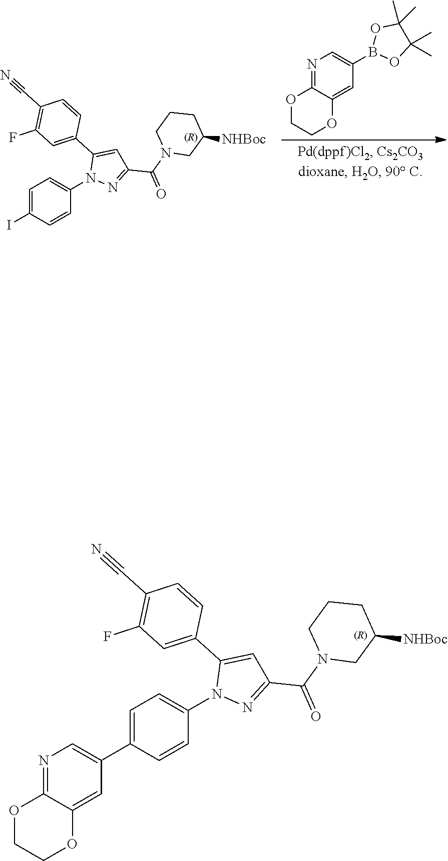

Tert-butyl (R)-(1-(5-(4-cyano-3-fluorophenyl)-1-(4-(2,3-dihydro-[1,4]dioxino[2,3-b]pyridin-7-yl)phenyl)-1H-pyrazole-3-carbonyl)piperidin-3-yl)carbamate. To a stirred solution of tert-butyl (R)-(1-(5-(4-cyano-3-fluorophenyl)-1-(4-iodophenyl)-1H-pyrazole-3-carbonyl)piperidin-3-yl)carbamate (702 mg, 1.14 mmol, 1.0 equiv) in dioxane (5 mL) and H₂O (0.5 mL) were added 7-(4,4,5,5-tetramethyl-1,3,2-dioxaborolan-2-yl)-2,3-dihydro-[1,4]dioxino[2,3-b]pyridine (300 mg, 1.14 mmol, 1.0 equiv), Pd(dppf)Cl₂-DCM (93 mg, 0.11 mmol, 0.1 equiv), and Cs₂CO₃ (929 mg, 2.85 mmol, 2.5 equiv) in portions at room temperature under a nitrogen atmosphere. The reaction mixture was stirred overnight at 90° C. under a nitrogen atmosphere. The mixture was allowed to cool down to room temperature. The resulting mixture was filtered, and the filter cake was washed with DCM (3×50 mL). The filtrate was concentrated under vacuum. The residue was purified by Prep-TLC (PE/EtOAc 1:3). This resulted in 300 mg (42%) of the title compound as a yellow solid. MS-ESI: 625 (M+1).

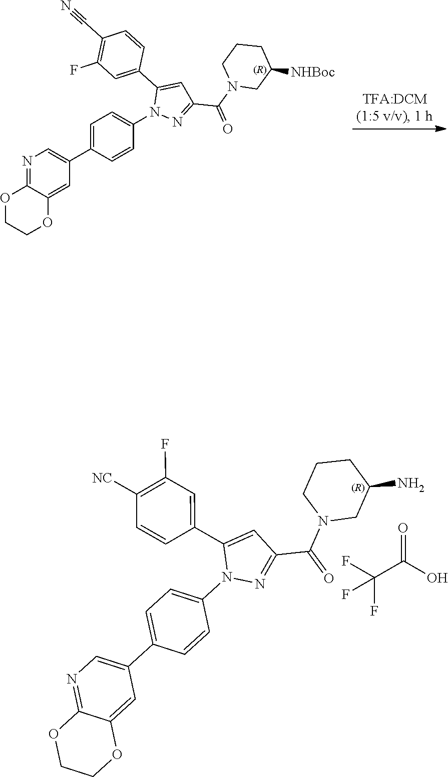

(R)-4-(3-(3-aminopiperidine-1-carbonyl)-1-(4-(2,3-dihydro-[1,4]dioxino[2,3-b]pyridin-7-yl)phenyl)-1H-pyrazol-5-yl)-2-fluorobenzonitrile 2,2,2-trifluoroacetate. A solution of tert-butyl (R)-(1-(5-(4-cyano-3-fluorophenyl)-1-(4-(2,3-dihydro-[1,4]dioxino[2,3-b]pyridin-7-yl)phenyl)-1H-pyrazole-3-carbonyl)piperidin-3-yl)carbamate (100 mg, 0.16 mmol, 1.0 equiv) in DCM (5 mL) and TFA (1 mL) was stirred for 1 h at room temperature. The mixture was basified to pH 10 with sat. Na₂CO₃ (aq.) and extracted with DCM (3×50 mL). The combined organic layers were dried over anhydrous Na₂SO₄ and concentrated under vacuum. The crude product was purified by Prep-HPLC with the following conditions: XBridge BEH C18 OBD Prep Column, 19*250 mm, 5 μm; Mobile Phase A: water (10 mM NH₄HCO₃), Mobile Phase B: ACN; Flow rate: 25 mL/min; Gradient: 40% B to 70% B in 6 min; Detector UV: 254 nm; Rₜ 1 (min): 5.02. This resulted in 30.3 mg (34.9%) of the title compound as a white solid. MS-ESI: 525 (M+1). ¹H NMR (400 MHz, MeOD-d₄) δ 8.05 (d, J=2.0 Hz, 1H), 7.79-7.69 (m, 3H), 7.64 (d, J=2.4 Hz, 1H), 7.47 (d, J=7.6 Hz, 2H), 7.41 (d, J=9.6 Hz, 1H), 7.29 (d, J=8.0 Hz, 1H), 7.15 (s, 1H), 4.76-4.58 (m, 1H), 4.55-4.37 (m, 3H), 4.36-4.25 (m, 2H), 3.83-3.58 (m, 1H), 3.57-3.35 (m, 2H), 2.25-2.09 (m, 1H), 1.97-1.85 (m, 1H), 1.84-1.66 (m, 2H).

Examples in the following table were prepared using similar conditions as described for those of Example 84 from the appropriate starting materials.

TABLE 7
Example Compounds.

| Ex. | | | |
|---|---|---|---|
| No. | Structure | Name | Data |
| 85 | 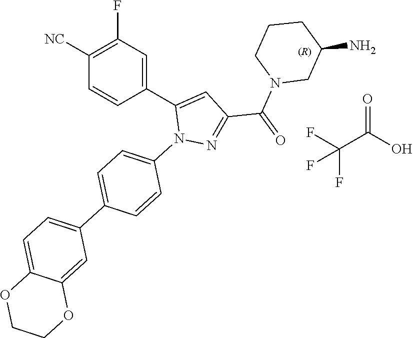 | (R)-4-(3-(3- aminopiperidine-1- carbonyl)-1-(4-(2,3- dihydrobenzo[b][1,4]dioxin- 6-yl)phenyl)-1H-pyrazol-5- yl)-2-fluorobenzonitrile 2,2,2-trifluoroacetate | MS-ESI: 524(M + 1) |

### Example 86

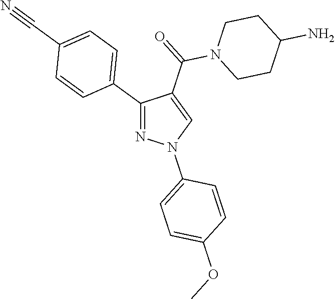 [SMILES: COc1ccc(-n2cc(C(=O)N3CCC(N)CC3)c(-c3ccc(C#N)cc3)n2)cc1]

### (R)-4-(3-(3-aminopiperidine-1-carbonyl)-1-(4-(2,3-dihydro-[1,4]dioxino[2,3-b]pyridin-7-yl)phenyl)-1H-pyrazol-5-yl)-2-fluorobenzonitrile 2,2,2-trifluoroacetate

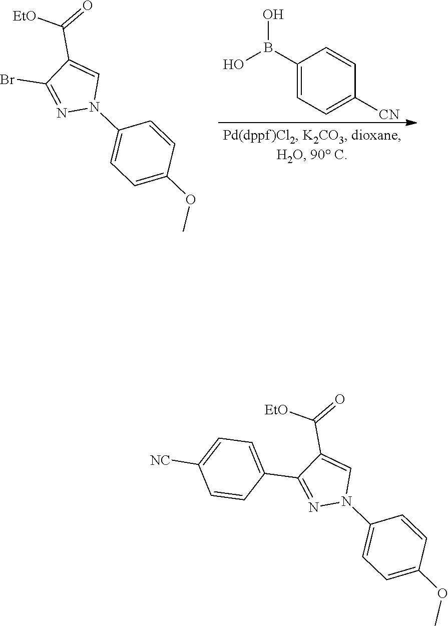 [SMILES: CCOC(=O)c1cn(-c2ccc(OC)cc2)nc1Br ; N#Cc1ccc(B(O)O)cc1 ; CCOC(=O)c1cn(-c2ccc(OC)cc2)nc1-c1ccc(C#N)cc1]

To a solution of ethyl 3-bromo-1-(4-methoxyphenyl)-1H-pyrazole-4-carboxylate (700 mg, 2.15 mmol, 1.00 equiv) and (4-cyanophenyl)boronic acid (475 mg, 3.23 mmol, 1.50 equiv) in dioxane (80 mL) and H₂O (8 mL) were added Pd(dppf)Cl₂ (315 mg, 0.43 mmol, 0.20 equiv) and K₂CO₃ (893 mg, 6.46 mmol, 3.00 equiv). After stirring for 2 h at 90° C. under a nitrogen atmosphere. The resulting mixture was concentrated under reduced pressure. The residue was purified by silica gel column chromatography, eluted with PE/EtOAc (6:1) to afford ethyl 3-(4-cyanophenyl)-1-(4-methoxyphenyl)-1H-pyrazole-4-carboxylate (600 mg, 80%) as a dark yellow solid.

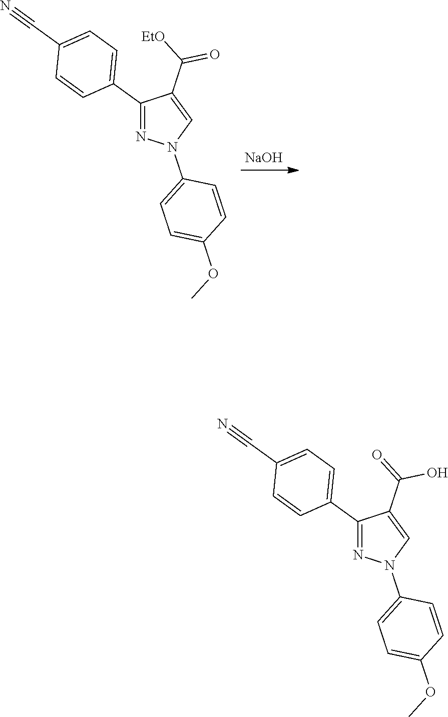

A solution of ethyl 3-(4-cyanophenyl)-1-(4-methoxyphenyl)-1H-pyrazole-4-carboxylate (600 mg, 1.73 mmol, 1.00 equiv) in THE (20 mL) and H₂O (5 mL) was added NaOH (276 mg, 6.91 mmol, 3.00 equiv) at room temperature. The resulting mixture was stirred for 1 h at room temperature. The mixture was acidified to pH 5 with HCl (1M). The resulting mixture was extracted with CH₂Cl₂ (3×100 mL). The combined organic layers were washed with NaCl sat. (2×50 mL) and dried over anhydrous Na₂SO₄. After filtration, the filtrate was concentrated under reduced pressure. This resulted in 3-(4-cyanophenyl)-1-(4-methoxyphenyl)-1H-pyrazole-4-carboxylic acid (470 mg, 85.2%) as a white solid.

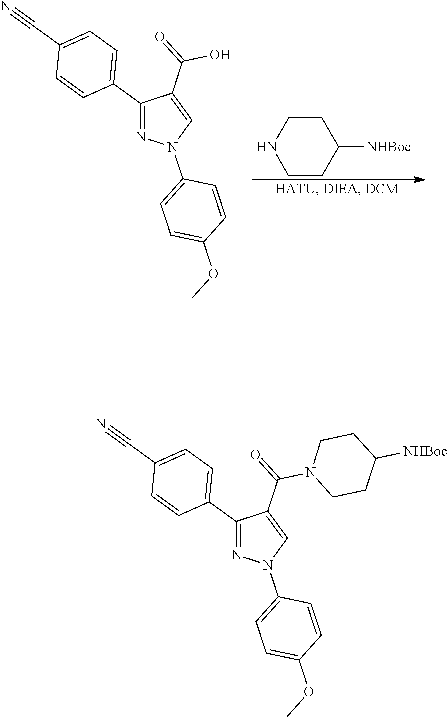

A solution of 3-(4-cyanophenyl)-1-(4-methoxyphenyl)-1H-pyrazole-4-carboxylic acid (300 mg, 0.94 mmol, 1.00 equiv) in DCM were added HATU (536 mg, 1.41 mmol, 1.50 equiv) and DIEA (364 mg, 2.82 mmol, 3.00 equiv). The mixture was stirred for 45 min at room temperature. To above stirred solution was added tert-butyl piperidin-4-ylcarbamate (282 mg, 1.41 mmol, 1.50 equiv). The resulting mixture was stirred for 30 min at room temperature. The resulting mixture was concentrated under reduced pressure. The residue was purified by silica gel column chromatography, eluted with hexane/EtOAc (5:1) to afford tert-butyl (1-(3-(4-cyanophenyl)-1-(4-methoxyphenyl)-1H-pyrazole-4-carbonyl)piperidin-4-yl)carbamate (260 mg, 55%) as a white solid.

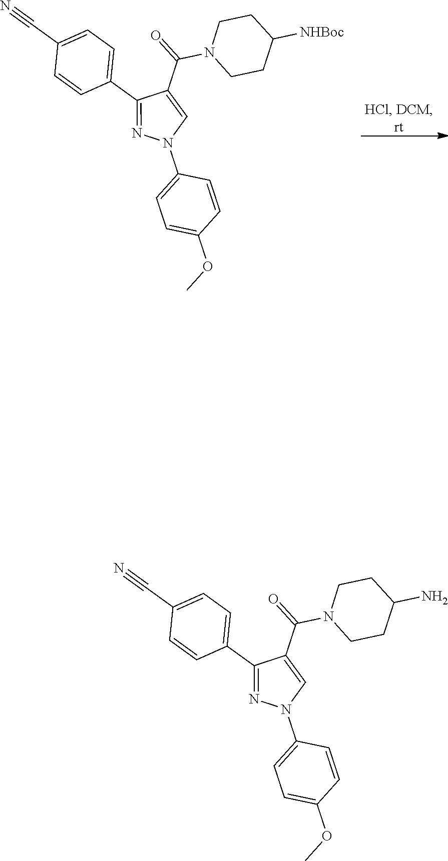 [SMILES: COc1ccc(-n2cc(C(=O)N3CCC(NC(=O)OC(C)(C)C)CC3)c(-c3ccc(C#N)cc3)n2)cc1 ; "HCl, DCM,rt" ; COc1ccc(-n2cc(C(=O)N3CCC(N)CC3)c(-c3ccc(C#N)cc3)n2)cc1]

A solution of tert-butyl (1-(3-(4-cyanophenyl)-1-(4-methoxyphenyl)-1H-pyrazole-4-carbonyl)piperidin-4-yl)carbamate (200 mg, 0.40 mmol, 1.00 equiv) in DCM (15 mL) was added HCl in dioxane (5 mL) at room temperature. The resulting mixture was stirred for 1 h at room temperature. The resulting mixture was concentrated under reduced pressure. The crude product was purified by Prep-HPLC with the following conditions: XBridge Prep OBD C18 Column 30×150 mm 5 μm; Mobile Phase A: water (10 mmol/L NH₄HCO₃), Mobile Phase B: ACN; Flow rate: 60 mL/min; Gradient: 30% B to 34% B in 7.5 min; 254/210 nm; Rₜ: 7.70 min to afford 4-(4-(4-aminopiperidine-1-carbonyl)-1-(4-methoxyphenyl)-1H-pyrazol-3-yl)benzonitrile (41.1 mg, 21.9%) as a white solid. MS-ESI: 402 [M+H]⁺. ¹H NMR (400 MHz, Methanol-d₄) δ ppm: 8.41 (s, 1H), 7.91 (d, J=8.4 Hz, 2H), 7.83-7.75 (m, 4H), 7.10-7.05 (m, 2H), 4.66-4.57 (s, 1H), 3.86 (s, 3H), 3.79-3.69 (m, 1H), 3.06-2.84 (m, 3H), 1.99-1.90 (m, 1H), 1.69-1.61 (m, 1H), 1.40-1.29 (m, 1H), 1.10-0.95 (m, 1H).

Examples in the following table were prepared using similar conditions as described for those of Example 86 from the appropriate starting materials.

TABLE 8
Example Compounds.

| Ex. | | | |
|---|---|---|---|
| No. | Structure | Name | Data |
| 87 | 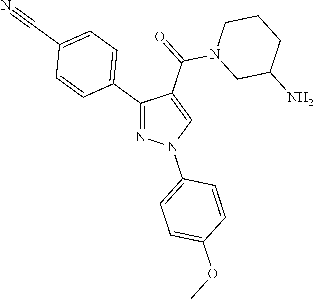 [SMILES: COc1ccc(-n2cc(C(=O)N3CCCC(N)C3)c(-c3ccc(C#N)cc3)n2)cc1] | 4-(4-(3-aminopiperidine-1- carbonyl)-1-(4- methoxyphenyl)-1H- pyrazol-3-yl)benzonitrile | MS-ESI: 402 (M + 1) |
| 88 | 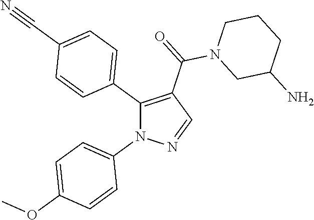 [SMILES: COc1ccc(-n2ncc(C(=O)N3CCCC(N)C3)c2-c2ccc(C#N)cc2)cc1] | 4-(4-(3-aminopiperidine-1- carbonyl)-1-(4- methoxyphenyl)-1H- pyrazol-5-yl)benzonitrile | MS-ESI: 402 (M + 1) |

### Example 89

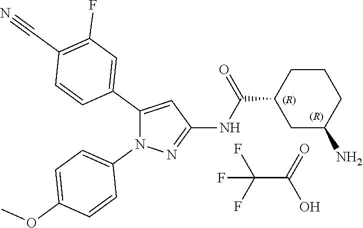

### (1R,3R)-3-amino-N-(5-(4-cyano-3-fluorophenyl)-1-(4-methoxyphenyl)-1H-pyrazol-3-yl)cyclohexane-1-carboxamide 2,2,2-trifluoroacetate

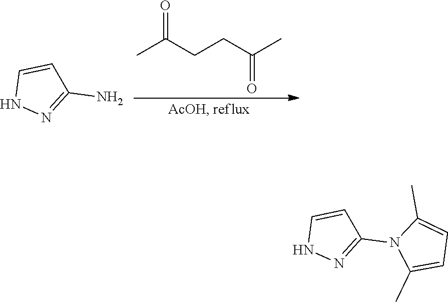

To a stirred solution of 1H-pyrazol-3-amine (20 g, 241 mmol, 1.00 equiv) in AcOH (200 mL) was added hexane-2,5-dione (20 mL) at room temperature. The reaction mixture was stirred for 12 h at 120° C. The mixture was allowed to cool down to room temperature and concentrated under vacuum. The resulting mixture was adjusted to pH 10-11 with sat. Na₂CO₃ aq. and extracted with EtOAc (3×250 mL). The combined organic layers were dried over anhydrous Na₂SO₄. After filtration, the filtrate was concentrated under vacuum. The residue was eluted from silica gel with PE/EtOAc (7:1). This resulted in 20 g (51.5%) of 3-(2,5-dimethyl-1H-pyrrol-1-yl)-1H-pyrazole as a yellow solid. MS-ESI: 162 (M+1).

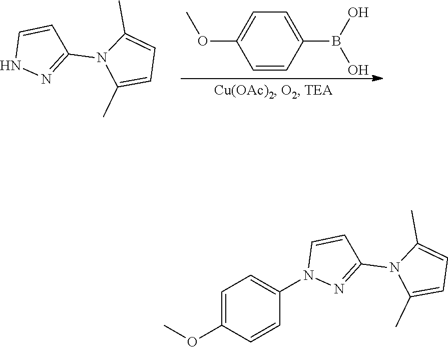 [SMILES: Cc1ccc(C)n1-c1cc[nH]n1 ; COc1ccc(B(O)O)cc1 ; COc1ccc(-n2ccc(-n3c(C)ccc3C)n2)cc1]

To a stirred solution of 3-(2,5-dimethyl-1H-pyrrol-1-yl)-1H-pyrazole (20 g, 124 mmol, 1.00 equiv) in DCM (500 mL) were added (4-methoxyphenyl)boronic acid (22.6 g, 149 mmol, 1.20 equiv), Cu(OAc)₂ (33.8 g, 186 mmol, 1.50 equiv) and TEA (37.7 g, 372 mmol, 3.00 equiv) at room temperature. The resulting mixture was stirred for 14 h at room temperature under O₂ atmosphere. The resulting mixture was concentrated under vacuum. The residue was eluted from silica gel with PE/EtOAc (9:1). After concentration under vacuum, the residue was purified by reverse flash chromatography with the following conditions: column, C18; Mobile Phase A: water (0.05% TFA), Mobile Phase B: ACN, 50% ACN to 80% ACN in 30 min, Detector UV 254 nm. This resulted in 7.4 g (22%) of 3-(2,5-dimethyl-1H-pyrrol-1-yl)-1-(4-methoxyphenyl)-1H-pyrazole as a yellow solid. MS-ESI: 268 (M+1).

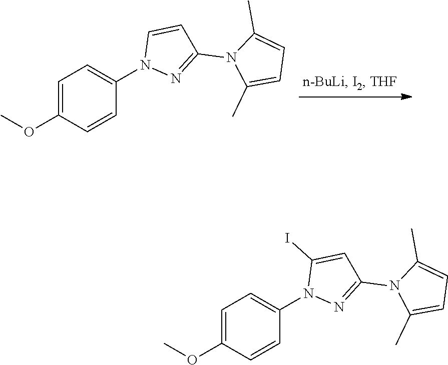 [SMILES: COc1ccc(-n2ccc(-n3c(C)ccc3C)n2)cc1 ; COc1ccc(-n2nc(-n3c(C)ccc3C)cc2I)cc1]

To a stirred solution of 3-(2,5-dimethyl-1H-pyrrol-1-yl)-1-(4-methoxyphenyl)-1H-pyrazole (7.4 g, 28 mmol, 1.00 equiv) in THE (200 mL) was added n-BuLi in hexane (2.5 M, 22.4 mL, 56 mmol, 2.00 equiv) dropwise at −78° C. under nitrogen atmosphere. The resulting mixture was stirred for 40 min at −78° C. under nitrogen atmosphere. To the above mixture was added I₂ (14 g, 56 mmol, 2.00 equiv) in THE (20 mL) dropwise at −78° C. The resulting mixture was stirred for additional 1 h at room temperature. The reaction was quenched with H₂O (200 mL) at room temperature and extracted with DCM (4×300 mL). The combined organic layers were washed with sat. NaCl aq. (500 mL). The combined organic layers were dried over anhydrous Na₂SO₄ and concentrated under vacuum. The residue was eluted from silica gel with PE/EtOAc (6:1). This resulted in 8.2 g (74.4%) of 3-(2,5-dimethyl-1H-pyrrol-1-yl)-5-iodo-1-(4-methoxyphenyl)-1H-pyrazole as a yellow solid. MS-ESI: 394 (M+1).

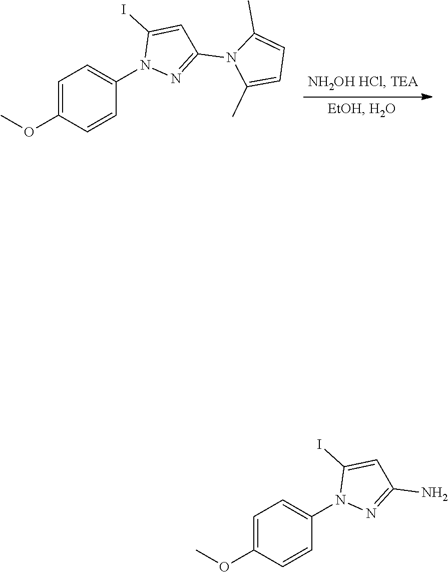 [SMILES: COc1ccc(-n2nc(-n3c(C)ccc3C)cc2I)cc1 ; COc1ccc(-n2nc(N)cc2I)cc1]

To a stirred solution of 3-(2,5-dimethyl-1H-pyrrol-1-yl)-5-iodo-1-(4-methoxyphenyl)-1H-pyrazole (8.2 g, 21 mmol, 1.00 equiv) in EtOH (80 mL) and H₂O (40 mL) were added TEA (12.7 g, 125 mmol, 6.00 equiv) and NH₂OH HCl (89 g, 1.25 mol, 60 equiv) at room temperature. The resulting mixture was stirred for 18 h at 95° C. The mixture was allowed to cool down to room temperature and acidified to pH 7 with NaOH (aq). (2 M). The resulting mixture was extracted with EtOAc (3×30 mL). The combined organic layers were washed with sat. NaCl aq. (2×40 mL) and dried over anhydrous Na₂SO₄. After filtration, the filtrate was concentrated under vacuum. This resulted in 6.1 g (92.8%) of 5-iodo-1-(4-methoxyphenyl)-1H-pyrazol-3-amine as yellow oil. MS-ESI: 316 (M+1).

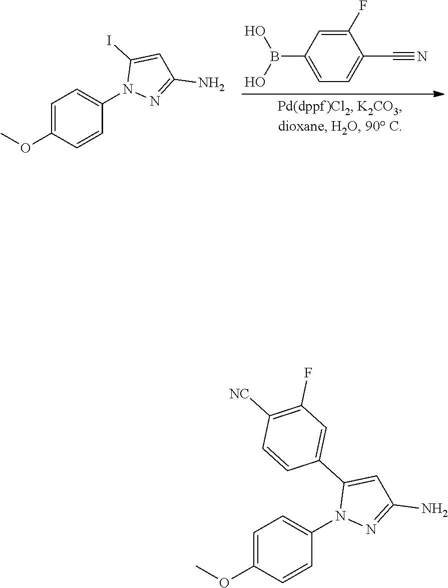 [SMILES: COc1ccc(-n2nc(N)cc2I)cc1 ; N#Cc1ccc(B(O)O)cc1F ; COc1ccc(-n2nc(N)cc2-c2ccc(C#N)c(F)c2)cc1]

To a stirred solution of 5-iodo-1-(4-methoxyphenyl)-1H-pyrazol-3-amine (6.1 g, 19.4 mmol, 1.00 equiv) in dioxane (100 mL) and H₂O (10 mL) were added 4-cyano-3-fluorophenylboronic acid (4.79 g, 29 mmol, 1.50 equiv), Pd(dppf)Cl₂ (2.83 g, 3.87 mmol, 0.20 equiv) and K₂CO₃ (8.03 g, 58.1 mmol, 3.00 equiv) under nitrogen at room temperature. The resulting mixture was stirred for 3 h at 90° C. under nitrogen atmosphere. The mixture was allowed to cool down to room temperature and concentrated under vacuum. The residue was eluted from silica gel with PE/EtOAc (1:1). This resulted in 5.4 g (90%) of 4-(3-amino-1-(4-methoxyphenyl)-1H-pyrazol-5-yl)-2-fluorobenzonitrile as a yellow solid. MS-ESI 309 (M+1).

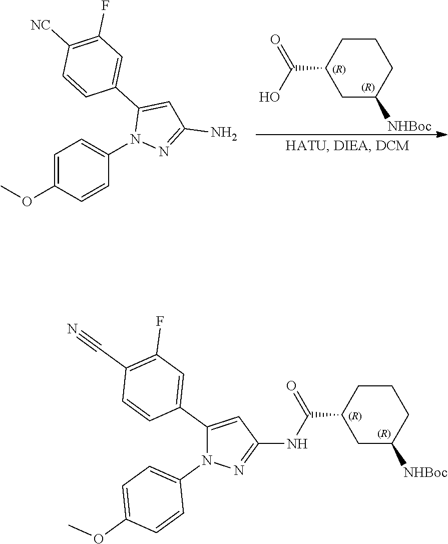

To a stirred solution of 4-(3-amino-1-(4-methoxyphenyl)-1H-pyrazol-5-yl)-2-fluorobenzonitrile (90 mg, 0.29 mmol, 1.00 equiv) in DCM (10 mL) were added (1R,3R)-3-((tert-butoxycarbonyl)amino)cyclohexane-1-carboxylic acid (107 mg, 0.44 mmol, 1.50 equiv), HATU (166 mg, 0.44 mmol, 1.50 equiv) and DIEA (113 mg, 0.88 mmol, 3.00 equiv) at room temperature. The resulting mixture was stirred for 1 h at room temperature. The resulting mixture was concentrated under vacuum. The residue was purified by Prep-TLC (PE/EtOAc 1:1). This resulted in 90 mg (57%) of tert-butyl ((1R,3R)-3-((5-(4-cyano-3-fluorophenyl)-1-(4-methoxyphenyl)-1H-pyrazol-3-yl)carbamoyl)cyclohexyl)carbamate as a white solid. MS-ESI: 534 (M+1).

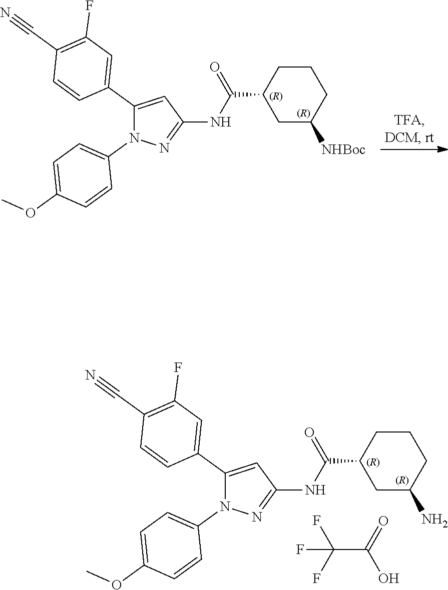

To a stirred solution of tert-butyl ((1R,3R)-3-((5-(4-cyano-3-fluorophenyl)-1-(4-methoxyphenyl)-1H-pyrazol-3-yl)carbamoyl)cyclohexyl)carbamate (80 mg, 0.15 mmol, 1.00 equiv) in DCM (20 mL) was added TFA (2 mL) dropwise at room temperature. The reaction mixture was stirred for 1 h at room temperature. The resulting mixture was concentrated under vacuum. The crude product was purified by Prep-HPLC with the following conditions: Column: Sunfire prep C18, 30*150*5 μm; Mobile Phase A: Water (0.05% TFA), Mobile Phase B: ACN; Flow rate: 60 mL/min; Gradient: 20% B to 44% B in 7 min; Detector UV 254 nm; Rₜ: 6.05 min. This resulted in 35 mg (42.6%) of (1R,3R)-3-amino-N-(5-(4-cyano-3-fluorophenyl)-1-(4-methoxyphenyl)-1H-pyrazol-3-yl)cyclohexane-1-carboxamide 2,2,2-trifluoroacetate as a white solid. MS-ESI: 434 (M+1). ¹H NMR (400 MHz, MeOD-d₄) δ 7.69 (dd, J=8.0, 6.8 Hz 1H), 7.26-7.17 (m, 4H), 7.05-6.94 (m, 3H), 3.84-3.75 (m, 4H), 2.98-2.96 (m, 1H), 2.28 (m, 1H), 2.00 (m, 2H), 1.79-1.55 (m, 4H), 1.51-1.37 (m, 1H).

Examples in the following table were prepared using similar conditions as described for those of Example 90 from the appropriate starting materials.

TABLE 9
Example Compounds.

| Ex. | | | |
|---|---|---|---|
| No. | Structure | Name | Data |
| 90 | 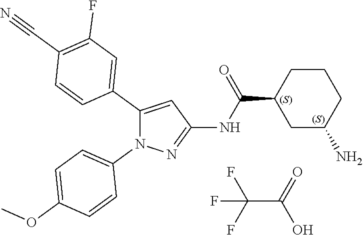 | (1S,3S)-3-amino-N-(5-(4- cyano-3-fluorophenyl)-1- (4-methoxyphenyl)-1H- pyrazol-3-yl)cyclohexane- 1-carboxamide 2,2,2- trifluoroacetate | MS-ESI: 434(M + 1) |
| 91 | 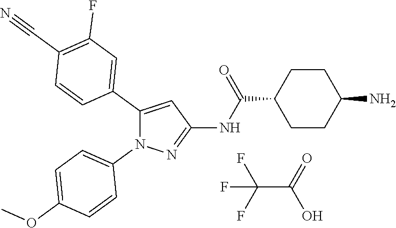 | (1R,4R)-4-Amino-N-(5-(4- cyano-3-fluorophenyl)-1- (4-methoxyphenyl)-1H- pyrazol-3-yl)cyclohexane- 1-carboxamide 2,2,2- trifluoroacetate | MS-ESI: 434(M + 1) |
| 92 | 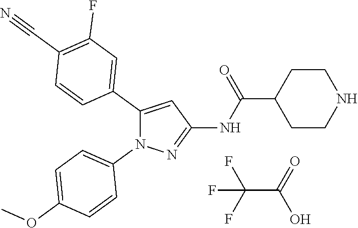 | N-(5-(4-cyano-3- fluorophenyl)-1-(4- methoxyphenyl)-1H- pyrazol-3-yl)piperidine-4- carboxamide 2,2,2- trifluoroacetate | MS-ESI: 420(M + 1) |
| 93 | 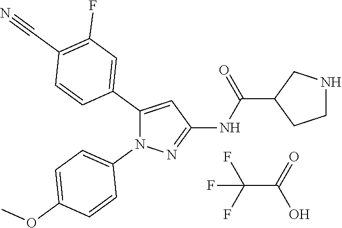 | N-(5-(4-cyano-3- fluorophenyl)-1-(4- methoxyphenyl)-1H- pyrazol-3-yl)pyrrolidine-3- carboxamide 2,2,2- trifluoroacetate | MS-ESI: 520(M + 1) |
| 94 | 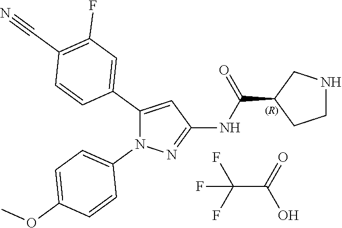 | (R)-N-(5-(4-cyano-3- fluorophenyl)-1-(4- methoxyphenyl)-1H- pyrazol-3-yl)pyrrolidine-3- carboxamide 2,2,2- trifluoroacetate | MS-ESI: 520(M + 1) |
| 95 | 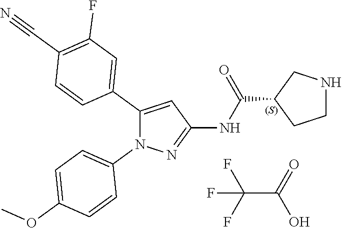 | (S)-N-(5-(4-cyano-3- fluorophenyl)-1-(4- methoxyphenyl)-1H- pyrazol-3-yl)pyrrolidine-3- carboxamide 2,2,2- trifluoroacetate | MS-ESI: 520(M + 1) |
| 96 | 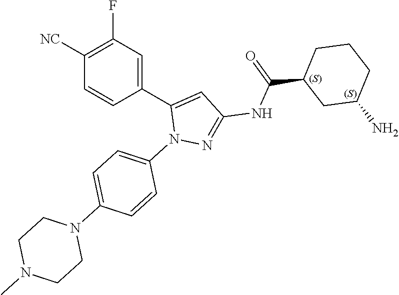 | (1S,3S)-3-amino-N-(5-(4- cyano-3-fluorophenyl)-1- (4-(4-methylpiperazin-1- yl)phenyl)-1H-pyrazol-3- yl)cyclohexane-1- carboxamide | MS-ESI: 502(M + 1) |

TABLE 10
Analytical data.

| Ex. | | |
|---|---|---|
| No. | LC-MS | ¹H NMR (400 MHz/MeOD-d₄, unless indicated otherwise) |
| 3 | MS-ESI: | 7.70 (d, J = 8.4 Hz, 2H), 7.43 (d, J = 8.4 Hz, 2H), 7.29-7.25 (m, 2H), 7.13 |
| | 402(M + 1) | (s, 1H), 7.04-6.99 (m, 2H), 4.33-4.26 (m, 1H), 3.83 (s, 3H), 3.52-3.47 (m, |
| | | 1H), 3.36-3.30 (m, 1H), 3.02-2.94 (m, 2H), 2.11-2.04 (m, 2H), 1.87-1.78 |
| | | (m, 2H). |
| 4 | MS-ESI: | 8.08 (dd, J = 8.4, 1.6 Hz, 2H), 7.79 (d, J = 8.4 Hz, 2H), 7.50 (t, J = 8.4 Hz, |
| | 402(M + 1) | 2H), 7.12 (s, 1H), 7.14-7.02 (m, 2H), 4.37 (d, J = 9.2 Hz, 1H), 3.86 (s, 3H), |
| | | 3.52-3.30 (m, 1H), 2.93-2.60 (m, 2H), 2.15-1.85 (m, 2H), 1.78-1.50 (m, |
| | | 1H), 1.39-1.02 (m, 2H) |
| 5 | MS-ESI: | 8.06 (d, J = 8.4 Hz, 2H), 7.82 (d, J = 8.4 Hz, 2H), 7.42 (d, J = 9.6 Hz, 2H), |
| | 402(M + 1) | 7.34 (s, 1H), 7.04 (d, J = 10.2 Hz, 2H), 4.08 (t, J = 10.5 Hz, 1H), 3.89 (s, |
| | | 3H), 3.56-3.42 (m, 1H), 3.38-3.36 (m, 1H), 3.00-2.82 (m, 2H), 2.09-2.04 |
| | | (m, 2H), 1.91-1.68 (m, 2H). |
| 6 | MS-ESI: | 7.71 (t, J = 7.5 Hz, 1H), 7.31-7.21 (m, 6H), 7.03 (s, 1H), 4.51-4.31 (m, |
| | 404(M + 1) | 2H), 3.18-3.01 (m, 1H), 2.90-2.78 (m, 2H), 2.38 (s, 3H), 2.04 (d, J = 10.8 |
| | | Hz, 1H), 1.87-1.82 (m, 1H), 1.66-1.59 (m, 1H), 1.53-1.39 (m, 1H) |
| 7 | MS-ESI: | 7.74 (t, J = 8.0 Hz, 1H), 7.30-7.21 (m, 6H), 7.04 (s, 1H), 4.87-4.38 (m, |
| | 418(M + 1) | 2H), 3.46-3.33 (m, 1H), 3.31-2.94 (m, 1H), 2.69-2.58 (m, 2H), 2.40 (s, |
| | | 3H), 1.98-1.89 (m, 1H), 1.79-1.76 (m, 2H), 1.63-1.58 (m, 1H), 1.42-1.37 |
| | | (m, 1H) |
| 8 | MS-ESI: | 7.74 (t, J = 8.0 Hz, 1H), 7.30-7.21 (m, 6H), 7.03 (s, 1H), 4.87-4.68 (m, |
| | 418(M + 1) | 2H), 3.30 (t, J = 12.8 Hz, 1H), 2.87 (t, J = 13 Hz, 1H), 2.59 (d, J = 6.8 Hz, |
| | | 2H), 2.38 (s, 3H), 1.92-1.70 (m, 3H), 1.32-1.18 (m, 2H) |
| 9 | MS-ESI: | 7.71 (t, J = 7.4 Hz, 1H), 7.29 (d, J = 8.7 Hz, 3H), 7.25-7.21 (m, 3H), 7.04 |
| | 404(M + 1) | (s, 1H), 4.80-4.62 (m, 2H), 3.25 (s, 1H), 3.09-2.91 (m, 2H), 3.40 (s, 3H), |
| | | 2.19-1.92 (m, 2H), 1.52-1.37 (m, 2H). |
| 10 | MS-ESI: | 7.69 (t, J = 8.0 Hz, 1H), 7.30-7.21 (m, 6H), 7.15 (s, 1H), 4.54-4.51 (m, |
| | 390(M + 1) | 1H), 3.16-3.06 (m, 2H), 2.95-2.84 (m, 2H), 2.40 (s, 3H), 2.23-2.17 (m, |
| | | 1H), 1.85-1.82 (m, 1H) |
| 11 | MS-ESI: | 7.74 (t, J = 7.4 Hz, 1H)), 7.33-7.23 (m, 6H), 7.06 (s, 1H), 4.95-4.35 (m, |
| | 404(M + 1) | 2H), 3.29-2.80 (m, 3H), 2.43 (s, 3H), 2.07-2.05 (m, 1H), 1.91-1.81 (m, |
| | | 1H), 1.64-1.58 (m, 1H), 1.50-1.44 (m, 1H) |
| 12 | MS-ESI: | 7.74 (t, J = 7.4 Hz, 1H)), 7.33-7.23 (m, 6H), 7.06 (s, 1H), 4.89-4.35 (m, |
| | 404(M + 1) | 2H), 3.34-2.80 (m, 3H), 2.43 (s, 3H), 2.07-2.04 (m, 1H), 1.91-1.81 (m, |
| | | 1H), 1.66-1.58 (m, 1H), 1.50-1.44 (m, 1H) |
| 13 | MS-ESI: | 7.92-7.78 (m, 3H), 7.46-7.33 (m, 4H), 7.16 (s, 1H), 4.48 (d, J = 13.2 Hz, |
| | 404(M + 1) | 1H), 3.57 (d, J = 14.1 Hz, 1H), 3.00-2.81 (m, 3H), 2.42 (s, 3H), 1.88 (d, J = |
| | | 12.1 Hz, 1H), 1.65 (d, J = 11.7 Hz, 1H), 1.30-1.16 (m, 1H), 0.98-0.92 (m, |
| | | 1H) |
| 14 | MS-ESI: | 7.89 (d, J = 10.2 Hz, 2H), 7.80 (t, J = 7.2 Hz, 1H), 7.46 (t, J = 8.9 Hz, 2H), |
| | 404(M + 1) | 7.35 (d, J = 8.1 Hz, 2H), 7.17 (s, 1H), 4.37 (d, J = 7.8 Hz, 1H), 3.51-3.38 |
| | | (m, 1H), 2.91-2.58 (m, 3H), 2.41 (s, 3H), 1.87-1.74 (m, 1H), 1.51-1.03 (m, |
| | | 3H) |
| 15 | MS-ESI: | 7.69 (t, J = 8.0 Hz, 1H)), 7.30-7.21 (m, 6H), 7.15 (s, 1H), 4.54-4.51 (m, |
| | 390(M + 1) | 1H), 3.16-3.06 (m, 2H), 2.95-2.84 (m, 2H), 2.40 (s, 3H), 2.23-2.17 (m, |
| | | 1H), 1.85-1.82 (m, 1H) |
| 16 | MS-ESI: | 7.71 (t, J = 7.4 Hz, 1H), 7.31-7.21 (m, 4H), 7.00 (t, J = 8.6 Hz, 3H), 4.51- |
| | 420(M + 1) | 4.32 (m, 2H), 3.83 (s, 3H), 3.31-2.78 (m, 3H), 2.05-2.01 (m, 2H), 1.87- |
| | | 1.35 (m, 2H). |
| 17 | MS-ESI: | 7.88 (d, J = 8.8 Hz, 2H), 7.79 (t, J = 7.2 Hz, 1H), 7.49 (t, J = 8.6 Hz, 2H), |
| | 420(M + 1) | 7.14 (d, J = 8.8 Hz, 1H), 7.11-7.03 (m, 2H), 4.35 (d, J = 9.6 Hz, 1H), 3.85 |
| | | (s, 3H), 3.52-3.33 (m, 1H), 2.90-2.60 (m, 2H), 2.17-2.03 (m, 1H), 1.88- |
| | | 1.84 (m, 1H), 1.78-1.74 (m, 1H), 1.52-0.98 (m, 3H) |
| 18 | MS-ESI: | 7.71 (t, J = 8.0 Hz, 1H), 7.69-7.22 (m, 4H), 7.00 (t, J = 8.2 Hz, 3H), 4.51- |
| | 420(M + 1) | 4.32 (m, 2H), 3.84 (s, 3H), 3.30-2.81 (m, 3H), 2.05-2.02 (m, 1H), 1.84- |
| | | 1.82 (m, 1H), 1.67-1.50 (m, 1H), 1.48-1.33 (m, 1H) |
| 19 | MS-ESI: | 7.72 (t, J = 8.0 Hz, 1H), 7.69-7.22 (m, 4H), 7.00 (t, J = 8.2 Hz, 3H), 4.86- |
| | 420(M + 1) | 4.32 (m, 2H), 3.84 (s, 3H), 3.30-2.81 (m, 3H), 2.05-2.02 (m, 1H), 1.84- |
| | | 1.82 (m, 1H), 1.67-1.50 (m, 1H), 1.48-1.33 (m, 1H) |
| 20 | MS-ESI: | (400 MHz, DMSO-d₆) 8.13 (d, J = 7.6 Hz, 1H), 7.92 (t, J = 7.4 Hz, 4H), |
| | 434(M + 1) | 7.50 (d, J = 5.8 Hz, 1H), 7.34 (d, J = 8.8 Hz, 2H), 7.28 (s, 1H), 7.20 (dd, J = |
| | | 8.4, 1.6 Hz, 1H), 7.04 (d, J = 7.2 Hz, 2H), 4.28 (s, 1H), 3.81 (s, 3H), 3.51 |
| | | (s, 1H), 1.99-1.94 (m, 1H), 1.79-1.73 (m, 1H), 1.66 (s, 4H), 1.59-1.55 (m, |
| | | 2H) |
| 21 | MS-ESI: | 7.71 (t, J = 8.0 Hz, 1H), 7.30-7.21 (m, 4H), 7.15 (s, 1H), 7.01 (d, J = 9.2 |
| | 406(M + 1) | Hz, 2H), 4.55-4.48 (m, 1H), 3.84 (s, 3H), 3.17-3.05 (m, 2H), 2.95-2.83 (m, |
| | | 1H), 2.21-2.18 (m, 2H), 1.83-1.81 (m, 2H) |
| 22 | MS-ESI: | 7.75 (t, J = 8.0 Hz, 1H), 7.33-7.24 (m, 4H), 7.17 (s, 1H), 7.08-7.01 (m, |
| | 434(M + 1) | 2H), 4.01-3.95 (m, 1H), 3.87 (s, 3H), 2.93-2.87 (m, 1H), 2.21-2.18 (m, |
| | | 1H), 1.98-1.87 (m, 3H), 1.50-1.47 (m, 1H), 1.36-1.26 (m, 2H), 1.18-1.14 |
| | | (m, 1H) |
| 23 | MS-ESI: | (400 MHz, DMSO-d₆) 8.13 (d, J = 7.6 Hz, 1H), 7.95-7.85 (m, 4H), 7.51 |
| | 434(M + 1) | (dd, J = 10.4, 1.6 Hz, 1H), 7.37-7.32 (m, 2H), 7.28 (s, 1H), 7.20 (dd, J = |
| | | 8.0, 1.6 Hz, 1H), 7.07-7.02 (m, 2H), 4.28 (s, 1H), 3.82 (s, 3H), 3.50 (s, |
| | | 1H), 2.00-1.93 (m, 1H), 1.77-1.49 (m, 7H) |
| 24 | MS-ESI: | 7.72 (t, J = 8.0 Hz, 1H), 7.69-7.21 (m, 4H), 7.15 (s, 1H), 7.06-7.00 (m, |
| | 420(M + 1) | 2H), 4.03-3.99 (m, 1H), 3.84 (s, 3H), 3.16-3.08 (m, 2H), 2.91-2.69 (m, |
| | | 2H), 1.96 (d, J = 2.8 Hz, 2H), 1.65-1.51 (m, 2H) |
| 25 | MS-ESI: | (400 MHz, DMSO-d₆) 8.13 (d, J = 7.6 Hz, 1H), 7.94-7.91 (m, 4H), 7.51 |
| | 434(M + 1) | (dd, J = 10.4, 1.6 Hz, 1H), 7.37-7.31 (m, 2H), 7.28 (s, 1H), 7.20 (d, J = 9.6 |
| | | Hz, 1H), 7.07-7.01 (d, J = 15.6 Hz, 2H), 4.28 (s, 1H), 3.81 (s, 3H), 3.51 (s, |
| | | 1H), 2.99-1.94 (m, 1H), 1.79-1.49 (m, 7H) |
| 26 | MS-ESI: | (400 MHz, DMSO-d₆) 8.62 (s, 1H), 8.35 (s, 1H), 7.92 (t, J = 7.4 Hz, 1H), |
| | 406(M + 1) | 7.50 (dd, J = 10.4, 1.6 Hz, 1H), 7.33 (d, J = 8.4 Hz, 2H), 7.28 (s, 1H), 7.20 |
| | | (d, J = 8 Hz, 1H), 7.01 (d, J = 8.4 Hz, 2H), 4.53 (s, 1H), 3.80 (s, 3H), 3.21 |
| | | (s, 2H), 3.03 (s, 2H), 2.11 (s, 1H), 1.87 (s, 1H) |
| 27 | MS-ESI: | (400 MHz, DMSO-d₆) 8.92 (s, 2H), 8.70 (s, 1H), 7.92 (t, J = 7.6 Hz, 1H), |
| | 406(M + 1) | 7.51 (dd, J = 10.4, 1.6 Hz, 1H), 7.37-7.31 (m, 2H), 7.29 (s, 1H), 7.20 (d, J = |
| | | 9.6 Hz, 1H), 7.07-7.00 (m, 2H), 4.63 (t, J = 6.2 Hz, 1H), 3.81 (s, 3H), |
| | | 3.45-3.23 (m, 2H), 3.22-3.19 (m, 2H), 2.26-2.17 (s, 1H), 2.05-1.98 (m, 1H) |
| 28 | MS-ESI: | 7.41 (t, J = 7.4 Hz, 1H), 7.33-7.29 (m, 3H), 7.25 (d, J = 8 Hz, 1H), 7.18 (s, |
| | 434(M + 1) | 1H), 7.04 (d, J = 8.8 Hz, 2H), 4.03 (m, 1H), 3.87 (s, 3H), 3.33-3.22 (m, |
| | | 1H), 2.34 (d, J = 11.2 Hz, 1H), 2.07-1.99 (m, 3H), 1.60-1.41 (m, 4H) |
| 29 | MS-ESI: | 7.89 (d, J = 7.6 Hz, 2H), 7.81 (t, J = 7.2 Hz, 1H), 7.46 (d, J = 8.8 Hz, 2H), |
| | 462(M + 1) | 7.17 (s, 1H), 7.10-7.04 (m, 2H), 4.19 (d, J = 10.8 Hz, 1H), 3.86 (s, 3H), |
| | | 3.58-3.43 (m, 2H), 3.18-3.10 (m, 2H), 2.04-2.01 (m, 1H), 1.78-1.50 (m, |
| | | 2H), 1.45-1.30 (m, 1H) |
| 30 | MS-ESI: | 7.72 (t, J = 7.4 Hz, 1H), 7.31-7.22 (m, 4H), 7.06 (s, 1H), 7.02 (t, J = 6.0 |
| | 462(M + 1) | Hz, 2H), 4.76 (t, J = 14.4 Hz, 1H), 4.61 (d, J = 13.6 Hz, 1H), 3.74-3.69 (m, |
| | | 1H), 3.42-3.30 (m, 1H), 3.08-3.02 (m, 1H), 2.10-2.02 (m, 2H), 1.68-1.45 |
| | | (m, 2H) |
| 31 | MS-ESI: | 7.71 (t, J = 7.4 Hz, 1H), 7.29-7.21 (m, 4H), 7.18 (s, 1H), 7.09-7.00 (m, |
| | 392(M + 1) | 2H), 5.02-4.79 (m, 1H), 4.68-4.64 (m, 1H), 4.55-4.51 (m, 1H), 4.23-4.15 |
| | | (m, 2H), 3.84 (s, 3H) |
| 32 | MS-ESI: | 7.71 (t, J = 7.6 Hz, 1H), 7.28-7.20 (m, 4H), 7.15 (s, 1H), 7.01 (d, J = 12.4 |
| | 406(M + 1) | Hz, 2H), 4.85-4.80 (m, 1H), 4.50-4.45 (m, 1H), 4.35 (t, J = 9.4 Hz, 1H), |
| | | 3.99-3.95 (m, 1H), 3.84 (s, 3H), 3.31-3.27 (m, 2H), 3.07-3.00 (m, 1H) |
| 33 | MS-ESI: | 7.88-7.83 (m, 1H), 7.48-7.42 (m, 2H), 6.97 (d, J = 8.7 Hz, 2H), 6.90 (s, |
| | 434(M + 1) | 1H), 6.86-6.83 (m, 2H), 5.51-5.48 (m, 2H), 4.45-4.35 (m, 2H), 3.97-3.92 |
| | | (m, 1H), 3.76 (s, 3H), 3.63-3.48 (m, 1H), 3.40-3.31 (m, 2H), 2.22-2.18 (m, |
| | | 1H), 1.88-1.76 (m, 3H) |
| 34 | MS-ESI: | 7.88-7.83 (m, 1H), 7.44-7.42 (m, 2H), 6.98-6.83 (m, 5H), 5.52-5.45 (m, |
| | 434(M + 1) | 2H), 4.42-4.34 (m, 2H), 4.00-3.95 (m, 1H), 3.76-3.50 (m, 4H), 3.40-3.32 |
| | | (m, 1H), 2.19-2.05 (m, 1H), 1.88-1.74 (m, 3H) |
| 35 | MS-ESI: | 7.73 (t, J = 7.4 Hz, 1H), 7.36-7.23 (m, 3H), 7.15 (t, J = 9.0 Hz, 1H), 7.17- |
| | 438(M + 1) | 7.02 (m, 2H), 4.85-4.32 (m, 2H), 3.90 (s, 3H), 3.16-3.01 (m, 1H), 2.88- |
| | | 2.81 (m, 2H), 2.05-2.12 (m, 1H), 1.88-1.79 (m, 1H), 1.62-1.59 (m, 1H), |
| | | 1.48-1.43 (m, 1H) |
| 36 | MS-ESI: | 7.75 (t, J = 6.4 Hz, 1H), 7.39-7.25 (m, 3H), 7.15 (t, J = 8.8 Hz, 1H), 7.09- |
| | 438(M + 1) | 7.04 (m, 2H), 4.89-4.49 (m, 2H), 3.94 (s, 3H), 3.34-3.31 (m, 1H), 3.18- |
| | | 3.06 (m, 1H), 2.89-2.82 (m, 1H), 2.08-2.04 (m, 1H), 1.87-1.86 (m, 1H), |
| | | 1.64-1.61 (m, 1H), 1.47-1.44 (m, 1H) |
| 37 | MS-ESI: | 7.75 (t, J = 8.4 Hz, 1H), 7.39-7.29 (m, 3H), 7.25 (t, J = 9.6 Hz, 1H), 7.19- |
| | 438(M + 1) | 7.04 (m, 2H), 4.53-4.49 (m, 2H), 3.94 (s, 3H), 3.37-3.32 (m, 1H), 3.16- |
| | | 3.13 (m, 1H), 2.89-2.82 (m, 1H), 2.08-2.04 (m, 1H), 1.87-1.86 (m, 1H), |
| | | 1.64-1.61 (m, 1H), 1.47-1.44 (m, 1H) |
| 38 | MS-ESI: | 7.86 (t, J = 7.2 Hz, 1H), 7.37 (t, J = 6.8 Hz, 2H), 6.71 (s, 1H), 5.84 (s, 1H), |
| | 423(M + 1) | 4.56 (s, 2H), 4.49-4.28 (m, 2H), 3.31 (s, 2H), 3.27-3.0 (m, 1H), 2.85-2.75 |
| | | (m, 2H), 2.16 (s, 3H), 2.05-2.03 (m, 1H), 1.89-1.77 (m, 1H), 1.63-1.54 (m, |
| | | 1H), 1.45-1.43 (m, 1H) |
| 39 | MS-ESI: | 7.87 (t, J = 6.8 Hz, 1H), 7.40 (t, J = 6.8 Hz, 2H), 6.71 (s, 1H), 5.85 (s, 1H), |
| | 423(M + 1) | 4.56 (s, 2H), 4.49-4.28 (m, 2H), 3.31 (s, 2H), 3.27-3.0 (m, 1H), 2.87-2.78 |
| | | (m, 2H), 2.16 (s, 3H), 2.05-2.03 (m, 1H), 1.89-1.77 (m, 1H), 1.63-1.54 (m, |
| | | 1H), 1.45-1.43 (m, 1H) |
| 40 | MS-ESI: | 8.30 (s, 1H), 7.73-7.67 (m, 3H), 7.37-7.24 (m, 2H), 7.24 (d, J = 1.2 Hz, |
| | 444(M + 1) | 1H), 7.09 (s, 1H), 4.89-4.53 (m, 1H), 4.37 (d, J = 13.6 Hz, 1H), 4.26 (s, |
| | | 3H), 2.23-3.06 (m, 1H), 2.94-2.84 (m, 2H), 2.08-2.06 (m, 2H), 1.91-1.90 |
| | | (m, 2H) |
| 41 | MS-ESI: | 8.38 (s, 1H), 7.94-7.75 (m, 5H), 7.58-7.53 (m, 1H), 7.19 (s, 1H), 4.35 (d, J = |
| | 444(M + 1) | 10.6 Hz, 1H), 4.26 (s, 3H), 3.55-3.11 (m, 1H), 2.90-2.56 (m, 3H), 2.10- |
| | | 1.70 (m, 2H), 1.47-1.17 (m, 2H) |
| 42 | MS-ESI: | 8.09 (s, 1H), 7.83 (s, 1H), 7.71-7.69 (m, 2H), 7.41 (dd, J = 8.0, 1.0 Hz, |
| | 444(M + 1) | 1H), 7.33 (d, J = 8.0 Hz, 1H), 7.22 (d, J = 8.0 Hz, 1H), 7.15 (s, 1H), 4.87- |
| | | 4.45 (m, 2H), 4.14 (s, 3H), 3.70-3.50 (m, 1H), 3.42-3.20 (m, 2H), 2.25- |
| | | 2.20 (m, 1H), 1.95-1.80 (brs, 1H), 1.70-1.60 (m, 2H). |
| 43 | MS-ESI: | 8.09 (s, 1H), 7.83 (brs, 1H), 7.72-7.65 (m, 2H), 7.41 (dd, J = 8.0, 2.0 Hz, |
| | 444(M + 1) | 1H), 7.32(d, J = 8.0 Hz, 1H), 7.22 (d, J = 8.0 Hz, 1H), 7.15 (s, 1H), 4.87- |
| | | 4.45 (m, 2H), 4.14 (s, 3H), 3.70-3.50 (m, 1H), 3.42-3.35 (m, 2H), 2.25- |
| | | 2.20 (m, 1H), 1.95-1.80 (brs, 1H), 1.70-1.60 (m, 2H). |
| 44 | MS-ESI: | 8.28 (s, 1H), 7.73 (t, J = 12.0 Hz, 3H), 7.38-7.21 (m, 3H), 7.13 (s, 1H), |
| | 444(M + 1) | 4.87-4.45 (m, 2H), 4.21 (s, 3H), 3.70-3.50 (m, 1H), 3.42-3.30 (brs, 2H), |
| | | 2.25-2.15 (m, 1H), 1.95-1.60 (m, 3H). |
| 46 | MS-ESI: | (400 MHz, DMSO-d₆) 7.96-7.87 (m, 1H), 7.55-7.46 (m, 1H), 7.26-7.03 (m, |
| | 487(M + 1) | 5H), 6.91-6.65 (m, 1H), 4.59-4.28 (m, 3H), 3.67 (t, J = 6.0 Hz, 2H), 3.58- |
| | | 3.43 (m, 1H), 3.21-3.04 (m, 2H), 3.03-2.84 (m, 1H), 2.83-2.54 (m, 2H), |
| | | 2.43 (t, J = 6.0 Hz, 2H), 2.37-1.95 (m, 1H), 1.94-1.75 (m, 2H), 1.74-1.59 |
| | | (m, 1H), 1.58-1.19 (m, 2H). |
| 47 | MS-ESI: | 7.74 (t, J = 7.6 Hz, 1H), 7.35-7.23 (m, 6H), 7.10 (s, 1H), 4.66-4.14 (m, |
| | 503(M + 1) | 2H), 3.77-3.64 (m, 1H), 3.50-3.39 (m, 6H), 2.21-2.16 (m, 1H), 2.05-1.65 |
| | | (m, 7H), 1.30 (s, 3H). |
| 48 | MS-ESI: | (400 MHz, DMSO-d₆) 7.75 (t, J = 4.0 Hz, 1H), 7.35-7.18 (m, 4H), 7.11 (s, |
| | 498(M + 1) | 1H), 7.09-7.03 (m, 2H), 4.77-4.05 (m, 2H), 3.84-3.61 (m, 1H), 3.59-3.49 |
| | | (m, 2H), 3.47-3.37 (m, 2H), 3.26-3.13 (m, 2H), 3.08-2.94 (m, 1H), 2.25- |
| | | 2.13 (m, 1H), 2.14-2.02 (m, 2H), 2.02-1.85(m, 3H), 1.83-1.67 (m, 2H). |
| 49 | MS-ESI: | 7.74 (t, J = 7.4 Hz, 1H), 7.38-7.18 (m, 4H), 7.09-6.95 (m, 3H), 4.58 (s, |
| | 488(M + 1) | 2H), 4.52-4.35 (m, 1H), 4.34-4.11 (m, 1H), 3.88 (s, 2H), 3.62-3.43 (m, |
| | | 2H), 3.26-3.08 (m, 3H), 2.17-2.06 (m, 1H), 1.96-1.79 (m, 1H), 1.75-1.54 |
| | | (m, 2H). |
| 50 | MS-ESI: | 7.61 (t, J = 7.4 Hz, 1H), 7.24-7.08 (m, 4H), 6.95-6.83 (m, 3H), 4.46-4.17 |
| | 502(M + 1) | (m, 2H), 3.80 (s, 2H), 3.55-3.39 (m, 4H), 3.19-2.96 (m, 1H), 2.93 (s, 3H), |
| | | 2.81-2.67 (m, 2H), 1.99-1.88 (m, 1H), 1.81-1.65 (m, 1H), 1.58-1.42 (m, |
| | | 1H), 1.41-1.26 (m, 1H). |
| 51 | MS-ESI: | 7.72 (t, J = 7.6 Hz, 1H), 7.46-7.33 (m, 1H), 7.29 (d, J = 8.8 Hz, 2H), 7.09 |
| | 518(M + 1) | (s, 1H), 6.68 (d, J = 8.8 Hz, 1H), 6.57 (d, J = 2.8 Hz, 1H), 4.71-4.07 (m, |
| | | 2H), 3.96 (s, 2H), 3.69-3.53 (m, 5H), 3.53-3.46 (m, 2H), 3.45-3.36 (m, |
| | | 1H), 3.20-2.90 (m, 2H), 2.25-2.10 (m, 1H), 2.01-1.84 (m, 1H), 1.81-1.64 |
| | | (m, 2H) |
| 53 | MS-ESI: | 8.13-7.98 (m, 1H), 7.72 (t, J = 7.4 Hz, 1H), 7.59-7.34 (m, 2H), 7.30 (dd, J = |
| | 504(M + 1) | 10.4, 1.6 Hz, 1H), 7.21 (d, J = 8.0 Hz, 1H), 7.11-7.05 (m, 1H), 4.73-4.02 |
| | | (m, 2H), 3.86-3.55 (m, 3H), 3.53-3.34 (m, 6H), 3.20-3.00 (m, 2H), 2.25- |
| | | 2.10 (m, 1H), 2.08-1.96 (m, 2H), 1.95-1.85 (m, 1H), 1.82-1.57 (m, 4H). |
| 54 | MS-ESI: | 8.19-8.06 (m, 1H), 7.76 (d, J = 7.6 Hz, 1H), 7.71-7.60 (s, 2H), 7.39-7.21 |
| | 489(M + 1) | (m, 2H), 7.09 (s, 1H), 4.74-4.33 (m, 2H), 4.23-3.87 (m, 2H), 3.82-3.54 (m, |
| | | 3H), 3.51-3.36 (m, 3H), 3.27-3.09 (m, 3H), 3.01 (s, 3H), 2.27-2.11 (m, |
| | | 1H), 2.01-1.86 (m, 1H), 1.85-1.67 (m, 2H). |
| 57 | MS-ESI: | 7.93 (d, J = 9.2 Hz, 2H), 7.89-7.81 (m, 1H), 7.55-7.44 (m, 2H), 7.03-7.19 |
| | 503(M + 1) | (m, 3H), 4.64-4.35 (m, 1H), 3.83-3.61 (m, 2H), 3.58-3.46 (m, 2H), 3.43 (s, |
| | | 3H), 3.25-2.95 (m, 4H), 2.93-2.67 (m, 1H), 2.16-2.01 (m, 3H), 1.98-1.66 |
| | | (m, 3H), 1.65-1.12 (m, 2H). |
| 58 | MS-ESI: | 7.93 (d, J = 9.2 Hz, 2H), 7.89-7.81 (m, 1H), 7.50 (d, J = 7.2 Hz, 2H), 7.24 |
| | 488(M + 1) | (s, 1H), 7.20 (d, J = 9.2 Hz, 2H), 4.63-4.32 (m, 1H), 4.19-3.38 (m, 8H), |
| | | 3.28-3.03 (m, 3H), 3.01 (s, 3H), 2.97-2.80 (m, 1H), 2.18-2.05 (m, 1H), |
| | | 1.97-1.71 (m, 1H), 1.70-1.19 (m, 2H). |
| 59 | MS-ESI: | 7.73 (t, J = 7.2 Hz, 1H), 7.37-7.17 (m, 4H), 7.06-7.00 (m, 3H), 4.54-4.35 |
| | 488(M + 1) | (m, 1H), 4.36 (d, J = 12.8 Hz, 1H), 3.32-3.26 (m, 4H), 2.90-2.75 (m, 1H), |
| | | 2.92-2.75 (m, 2H), 2.63 (t, J = 4.8 Hz, 4H), 2.37 (s, 3H), 2.17-2.01 (m, |
| | | 1H), 1.95-1.77 (m, 1H), 1.70-1.57 (m, 1H), 1.51-1.36 (m, 1H). |
| 60 | MS-ESI: | 7.73 (t, J = 7.4 Hz, 1H), 7.39-7.12 (m, 4H), 7.08-6.97 (m, 3H), 4.59-4.36 |
| | 488(M + 1) | (m, 2H), 3.31-3.25 (m, 4H), 3.21-2.97 (m, 1H), 2.93-2.73 (m, 2H), 2.63 (t, |
| | | J = 5.2 Hz, 4H), 2.37 (s, 3H), 2.11-2.01 (m, 1H), 1.93-1.77 (m, 1H), 1.69- |
| | | 1.55 (m, 1H), 1.52-1.38 (m, 1H) |
| 61 | MS-ESI: | 7.73 (t, J = 8.0 Hz, 1H), 7.29 (t, J = 8.0 Hz, 2H), 7.20-7.04 (m, 3H), 6.62 |
| | 459(M + 1) | (d, J = 8.8 Hz, 2H), 4.77-4.10 (m, 2H), 3.83-3.56 (m, 1H), 3.53-3.50 (m, |
| | | 2H), 3.37-.34 (m, 4H), 2.25-2.13 (m, 1H), 2.11-2.05 (m, 4H), 2.0-1.85 (m, |
| | | 1H), 1.85-1.68 (m, 2H). |
| 62 | MS-ESI: | 7.73 (t, J = 8.0 Hz, 1H), 7.33-7.23 (m, 2H), 7.20-6.98 (m, 3H), 6.62 (d, J = |
| | 459(M + 1) | 9.2 Hz, 2H), 4.90-4.03 (m, 2H), 3.88-3.61 (m, 1H), 3.52-3.35 (m, 6H), |
| | | 2.26-2.11 (m, 1H), 2.11-1.99 (m, 4H), 1.99-1.85 (m, 1H), 1.85-1.59 (m, |
| | | 2H). |
| 63 | MS-ESI: | 7.76 (dd, J = 6.8, 6.4 Hz, 1H), 7.51-7.22 (m, 6H), 7.12 (s, 1H), 4.79-4.10 |
| | 503(M + 1) | (m, 2H), 3.82-3.50 (m, 4H), 3.42 (s, 3H), 3.32-3.23 (m, 4H), 2.26-2.07 (m, |
| | | 3H), 1.95-1.60 (m, 5H) |
| 64 | MS-ESI: | 7.75 (t, J = 6.8 Hz, 1H), 7.41-7.21 (m, 6H), 7.11 (s, 1H), 4.95-4.35 (m, |
| | 503(M + 1) | 2H), 3.81-3.61 (m, 3H), 3.60-3.49 (m, 1H), 3.48-3.35 (m, 5H), 3.31-3.20 |
| | | (m, 2H), 2.26-2.06 (m, 3H), 1.98-1.66 (m, 5H). |
| 65 | MS-ESI: | 8.05 (s, 1H), 7.88 (s, 1H), 7.75 (dd, J = 7.6, 6.8 Hz, 1H), 7.67 (d, J = 8.4 |
| | 470(M + 1) | Hz, 2H), 7.41-7.31 (m, 3H), 7.27 (d, J = 8.0 Hz, 1H), 7.13 (s, 1H), 4.91- |
| | | 4.21 (m, 2H), 3.95 (s, 3H), 3.84-3.58 (m, 1H), 3.42 (s, 2H), 2.37-2.13 (m, |
| | | 1H), 1.97-1.86 (m, 1H), 1.86-1.66 (m, 2H) |
| 66 | MS-ESI: | 8.05 (s, 1H), 7.88 (s, 1H), 7.75 (dd, J = 8.0, 7.2 Hz, 1H), 7.68 (d, J = 8.4 |
| | 470(M + 1) | Hz, 2H), 7.42-7.31 (m, 3H), 7.28 (d, J = 8.0 Hz, 1H), 7.13 (s, 1H), 4.77- |
| | | 4.10 (m, 2H), 3.95 (s, 3H), 3.85-3.55 (m, 1H), 3.42 (s, 2H), 2.27-2.13 (m, |
| | | 1H), 1.99-1.85 (m, 1H), 1.84-1.65 (m, 2H) |
| 67 | MS-ESI: | 7.76 (t, J = 7.6 Hz, 1H), 7.61-7.45 (m, 1H), 7.34-7.28 (m, 2H), 7.14 (s, |
| | 506(M + 1) | 1H), 7.00 (d, J = 8.8 Hz, 1H), 6.94(d, J = 13.6 Hz 1H), 4.56-4.11 (m, 2H), |
| | | 4.09-3.73 (m, 3H), 3.71-3.39 (m, 4H), 3.35-3.22 (m, 2H), 3.16-2.95 (m, |
| | | 2H), 2.87 (s, 3H), 2.13-2.01 (m, 1H), 1.86-1.72 (m, 1H), 1.71-1.53 (m, |
| | | 2H). |
| 68 | MS-ESI: | 7.72 (t, J = 7.0 Hz, 1H), 7.54-7.38 (m, 1H), 7.29 (dd, J = 19.6, 8.0 Hz, 2H), |
| | 520(M + 1) | 7.06 (s, 1H), 6.90-6.72 (m, 2H), 4.53-4.26 (m, 2H), 3.94 (s, 2H), 3.62 (d, J = |
| | | 5.6 Hz 2H), 3.54 (d, J = 6.8 Hz, 2H), 3.26-3.21 (m, 1H), 3.18-3.07 (m, |
| | | 1H), 3.03 (s, 3H), 2.91-2.75 (m, 1H), 2.07-1.98 (m, 1H), 1.91-1.75 (m, |
| | | 1H), 1.66-1.52 (m, 1H), 1.53-1.35 (m, 1H) |
| 80 | MS-ESI: | 7.96-7.80 (m, 3H), 7.43 (dd, J = 12.4, 1.2 Hz, 1H), 7.37-7.30 (m, 1H), |
| | 506(M + 1) | 7.29-7.19 (m, 2H), 4.60-4.35 (m, 1H), 4.05-3.51 (m, 5H), 3.49-3.34 (m, |
| | | 2H), 3.30-3.05 (m, 5H), 2.99 (s, 3H), 2.22-2.11 (m, 1H), 1.99-1.70 (m, |
| | | 1H), 1.69-1.35 (m, 2H). |
| 85 | MS-ESI: | 7.77 (t, J = 7.4 Hz, 1H), 7.69 (d, J = 8.4 Hz, 2H), 7.41 (d, J = 8.8 Hz, 3H), |
| | 524(M + 1) | 7.28 (d, J = 8.0 Hz, 1H), 7.18-7.12 (m, 3H), 6.94 (d, J = 8.0 Hz, 1H), 4.78- |
| | | 4.62 (m, 1H), 4.55-4.36 (m, 1H), 4.30 (s, 4H), 3.83-3.61 (m, 1H), 3.53- |
| | | 3.37 (m, 2H), 2.28-2.12 (m, 1H), 2.01-1.86 (m, 1H), 1.84-1.69 (m, 2H). |
| 87 | MS-ESI: | 8.45 (s, 1H), 7.91 (d, J = 8.4 Hz, 2H), 7.83-7.76 (m, 4H), 7.10-7.06 (m, |
| | 402(M + 1) | 2H), 4.72-4.29 (m, 1H), 3.86 (s, 3H), 3.72-3.43 (m, 1H), 3.21-2.72 (m, |
| | | 3H), 2.17-2.06 (m, 1H), 1.98-1.63 (m, 1H), 1.61-1.50 (m,1H), 1.43-1.20 |
| | | (m, 1H). |
| 88 | MS-ESI: | 7.87 (s, 1H), 7.73-7.70 (m, 2H), 7.39 (d, J = 8.4 Hz, 2H), 7.22-7.17 (m, |
| | 402(M + 1) | 2H), 6.97-6.93 (m, 2H), 4.48-4.19 (m, 1H), 3.80 (s, 3H), 3.71-3.51 (m, |
| | | 1H), 2.94-2.71 (m, 1H), 2.69-2.51 (m,1H), 2.18-0.89 (m, 5H). |
| 90 | MS-ESI: | 7.69 (t, J = 7.6 Hz, 1H), 7.24-7.19 (m, 4H), 7.02-6.96 (m, 3H), 3.90-3.78 |
| | 434(M + 1) | (m, 4H), 3.32-3.30 (m, 1H), 2.28-2.20 (m, 1H), 2.00-1.95 (m, 2H), 1.78- |
| | | 1.62 (m, 4H), 1.51-1.37 (m, 1H). |
| 91 | MS-ESI: | 7.69 (t, J = 7.6 Hz, 1H), 7.23-7.20 (m, 4H), 7.03 (s, 1H), 6.99 (t, J = 6.2 |
| | 434(M + 1) | Hz, 2H), 3.83 (s, 3H), 3.16-3.10 (m, 1H), 2.48-2.42 (m, 1H), 2.15-2.05 (m, |
| | | 4H), 1.75-1.61 (m, 2H), 1.51-1.37 (m, 2H). |
| 92 | MS-ESI: | 7.69 (t, J = 8.8 Hz, 1H), 7.25-7.17 (m, 4H), 7.04 (s, 1H), 7.00-6.94 (m, |
| | 420(M + 1) | 2H), 3.83 (s, 3H), 3.52-3.47 (m, 2H), 3.12-3.05 (m, 2H), 2.80-2.75 (m, |
| | | 1H), 2.15-2.10 (m, 2H), 2.06-1.94 (m, 2H). |
| 93 | MS-ESI: | 7.69 (dd, J = 8.0, 6.8 Hz, 1H), 7.25-7.17 (m, 4H), 7.06 (s, 1H), 7.03-6.95 |
| | 520(M + 1) | (m, 2H), 3.83 (s, 3H), 3.67-3.63 (m, 1H), 3.50-3.30 (m, 4H), 2.47-2.35 (m, |
| | | 1H), 2.32-2.21 (m, 1H). |
| 94 | MS-ESI: | 7.68 (dd, J = 6.4, 1.6 Hz, 1H), 7.25-7.17 (m, 4H), 7.06 (s, 1H), 6.99-6.95 |
| | 520(M + 1) | (m, 2H), 3.83 (s, 3H), 3.68-3.64 (m, 1H), 3.51-3.41 (m, 4H), 2.45-2.31 (m, |
| | | 1H), 2.29-2.24 (m, 1H). |
| 95 | MS-ESI: | 7.69 (dd, J = 8.0, 6.4 Hz, 1H), 7.25-7.17 (m, 4H), 7.05 (s, 1H), 7.00-6.94 |
| | 520(M + 1) | (m, 2H), 3.83 (s, 3H), 3.67-3.63 (m, 1H), 3.51-3.40 (m, 4H), 2.45-2.31 (m, |
| | | 1H), 2.29-2.24 (m, 1H). |
| 96 | MS-ESI: | N/A |
| | 502(M + 1) | |

### Biological Activity Assay

The activity of the Examples above may be illustrated in the following assays. Compounds listed above, which may not yet have been made and/or tested, are predicted to have activity in these assays.

Assaying the inhibition of KDM1A can be determined in vitro, in cultured cells, and in animals. There are a variety of spectrophotometric methods to detect the results of demethylation of methylated lysines, viz., detecting the products of KDM1A demethylase oxidative activity on a peptide fragment of at least 18 amino acids representing the N-terminus of the histone H3 substrate that contains a monomethyl at the fourth lysine residue. Hydrogen peroxide, one product of the KDM1A demethylase reaction, reacts with horseradish peroxidase and dihydroxyphenoxazine (ADHP) to produce the fluorescent compound resorufin (excitation=530-560 nm; emission=590 nm). The KDM1A demethylase enzyme activity can be obtained from mammalian cells or tissues expressing KDM1A from an endogenous or recombinant gene and purified or assayed from a whole cell extract. These methods can be used to determine the concentration of the disclosed compounds can inhibit fifty percent of the enzyme activity (IC₅₀). In one aspect, the disclosed compounds exhibit inhibition fifty percent of the KDM1A enzyme activity at a concentration of less than 500 nM, less than 100 nM, less than 50 nM, or less than 10 nM.

The association of KDM1A with other proteins can be determined by a variety of both in vitro and in vivo methods known to one skilled in the art. For example, the disruption of KDM1A with associated proteins can be determined in an electromobility shift assay (EMSA). In various aspects, the disruption of the physical association of KDM1A with CoREST protein by the disclosed compounds can be observed using EMSA. In another example, the disruption of KDM1A with associated proteins can be determined by immunoprecipitation followed by separation of the co-precipitated proteins by mass spectroscopy or by get electrophoresis. In another example, the disruption of KDM1A association with CoREST can be determined by the ability of KDM1A to act on a nucleosomal substrate containing K4 or K9 methylated histone H3, a substrate that requires the presence of both KDM1A and CoREST. The disclosed compounds could be used to assay inhibition of CoREST association with KDM1A using nucleosomal substrate; such compounds may not inhibit KDM1A enzymatic activity as determined by the use of the histone H3 K4 methylated peptide substrate.

The inhibition of KDM1A can be determined in a cell-based assay. For example, KDM1A is an essential enzyme and prolonged inhibition of KDM1A will result in cell death, thus cell growth inhibition, arrest of cell growth or cell death can be assayed. In another aspect, genes induced by androgens and estrogens require KDM1A activity; inhibition by the disclosed compounds of KDM1A will abrogate the induction of gene expression in cells treated with androgens or estrogens. These effects can be measured, e.g., using quantitative PCR of mRNA to measure the magnitude of gene expression for androgen- and estrogen-dependent genes. KDM1A activity is required for the repression of transcription of specific genes. Inhibition of KDM1A by the disclosed compounds could de-repress the expression such genes in cell. These genes include MEIS1, CD86, VEG-A, AIM1, HMOX1, VIM, SKAP1, BMP, EOMES, FOXA2, HNF4, SOX17, GH, PSA, pS2, GREB1, GR-1b, PRL, TSHB, SYN1, HBG, SCN1A, SCN2a, and SCN3A, the expression of which can be assayed using quantitative PCR of mRNA before and at various times following the treatment of cells with the disclosed compounds. In another aspect, KDM1A is a regulator of leukemic stem cell potential and is required for oncogenic transformation of myeloid cells to acute myeloid leukemia (AML) by MLL-AF9. Inhibition of KDM1A in MLL-AF9-transformed cells grown in culture overcomes the arrest in differentiation to resulting in a more mature cell expressing the CD11b surface antigen, a monocytic cell antigen. Thus, inhibition of KDM1A can be assayed using an AML cell line such as THP-1 grown in culture quantifying the proportion of cells newly expressing the CD11b antigen using fluorescence activated cell sorting (FACS). A similar assay using FACS to count cells displaying the CD14 or CD86 can be also used, each of which are characteristic of more mature cells along the macrophage/monocytic lineage. Other cells lines derived from patients with acute myeloid leukemia such as MV4;11 or MOLM-13 cells can be used for this assay. Other markers of differentiation along the macrophage/monocyte lineage can be similarly assayed by FACS such as CD14 and CD86. Other AML cell lines such as MPLM-13 or MV4;11 can be assayed for the induction of either specific genes mentioned above or the differentiation markers as well as cell growth or apoptosis by Annexin V staining and FACS enumeration.

The selectivity of the disclosed compounds for KDM1A can be determined by assaying the IC₅₀ of the disclosed compounds for other FAD-dependent aminoxidases such as monoamine oxidase A (MAO-A), monoamine oxidase B (MAO-B), IL4I1, KDM1B, or SMOX. As such, a disclosed compound would inhibit KDM1A with an IC₅₀ that is 50-fold, or 100-fold, or 250-fold, or 500-fold less than for MAO-A or MAO-B.

The histone demethylase assay can be performed essentially as described in Shi, Y. et al. Cell 199, 941-953 (2004). Briefly, bulk histones, histone peptides or nucleosomes are incubated with purified human recombinant KDM1A, in the histone demethylase activity (HDM) assay buffer 1 (50 mM Tris pH 8.5, 50 mM KCl, 5 mM MgCl, 0.5% BSA, and 5% glycerol) from 30 minutes to 4 hours at 37° C. A typical reaction is conducted in 100 microliters in which either 20 micrograms of purified bulk histones or 3 micrograms of modified histone peptides are used as substrates. Different amounts of KDM1A ranging from 1-20 micrograms are used in the reaction along with, as necessary, other co-factors such as FAD or CoREST, depending on the chosen substrate. The reaction mixture is analyzed by SDS-PAGE and Western blotting using histone methyl-specific antibodies or by formaldehyde formation assay to examine the removal and conversion of the methyl group to formaldehyde, or by mass spectrometry in the case of peptide substrates to identify the demethylated histone peptide.

Bulk histones (e.g., 4 mg) are incubated with the indicated amounts of recombinant proteins or complexes in histone demethylase (HDM) assay buffer A (50 mM Tris pH 8.5, 50 mM KCl, 5 mM MgCl, 5% glycerol, 0.2 mM phenylmethylsulphonyl fluoride, and 1 mM dithiothreitol) in a final volume of 10 mL for 12-16 h at 37° C. For nucleosomes (0.3 mg) or mononucleosome (0.3 mg), HDM buffer A containing 0.1% NP40 can be used. The reaction mixture can then be analyzed by SDS-PAGE followed by Western blotting. Antibodies against mono- or di-methyl K4 in histone H3 and acetyl-K9/K14 of histone H3 are used to detect the degree of methylation and acetylation, respectively. Western blots are then quantified by densitometry or by intensity of luminescence.

Alternatively, a standard fluorogenic assay can be used in which the methylated histone substrate is tethered to the bottom of a 96 well plate (or to beads resting in the plate) using biotin conjugated to the histone methylated substrate and streptavidin (SA) on beads or SA attached to the plate to secure the biotinylated substrate. After incubation of the KDM1A enzyme in histone demethylase buffer A, the demethylated histone substrate can be detected using antibodies specific for demethylated H3K4 substrate conjugated to a fluor or some other agent that can be detected. A variation on that assay method would employ an antibody directed against the methylated version of the histone in which the amount of substrate is quantified before and after incubation with the enzyme. Yet another version of a similar assay would employ a fluorescence resonance energy transfer (FRET) system of detection in which the antibody recognizing the methylated version is conjugated or otherwise linked to an entity, e.g., a bead or a large carrier molecule on which a fluorophore (donor) is attached, and the fluorophore (acceptor) is bound to an entity linked to the substrate.

Alternatively, the production of H₂O₂ during the KDM1A reaction can be detected fluorometrically. In this system, the production of H₂O₂ is detected in the HDM assay buffer after exposure to substrate, co-factor and enzyme using ADHP (10-Acetyl-3, 7-dihydroxyphenoxazine) as a fluorogenic substrate for horse radish peroxidase (HRP). ADHP (also known as Amplex Red Reagent) is the most stable and sensitive fluorogenic substrate for HRP. The florescent product is resorufin. Sensitivity can be as low as 10⁻¹⁵ M of target protein. The signal is read using a fluorescence microplate reader at excitation and emission wavelengths of 530-560 nm and 590 nm, respectively.

Additionally, the KDM1A reaction can include other factors which may influence the activity of KDM1A. Such factors might include CoREST, NuRD complexes, DNMT1, HDAC1, HDAC2, and HDAC3, for example, as proteins known to associate with KDM1A or KDM1A-containing complexes. Interactions that influence any aspect of the KDM1A activity including specificity for template, substrate, Kₘ, K_{cat}, or sensitivity to FAD concentrations can be assayed. For example, an in vitro interaction assay between KDM1A and CoREST can be performed adding recombinant KDM1A (e.g., 10 mg) and CoREST (e.g., 5 mg) mixed and incubated for 1 h at 4-8° C., fractionated by Superdex 200 gel filtration column in a buffer containing 20 mM Tris-HCl pH 7.9, 500 mM KCl, 10% glycerol, 0.2 mM EDTA, 1 mM dithiothreitol, 0.1% Nonidet P40 and 0.2 mM phenylmethylsulphonyl fluoride, and then analyzed by silver staining.

For co-immunoprecipitation of mononucleosomes with KDM1A and CoREST, nucleosomes (1.5 mg) can be digested with micrococcal nuclease and incubated with recombinant KDM1A (e.g., 1 mg), CoREST (e.g., 500 ng), or both proteins in HDM buffer A containing 0.1% NP40 for 1 h at 4-8° C. Antibodies directed against KDM1A or CoREST attached to an affinity resin are added and after extensive washing with HDM buffer A containing 0.1% NP40, the bound proteins are eluted with a wash buffer. KDM1A activity can be assayed in the eluate or the concentration of KDM1A can be determined by quantitative Western blotting.

Compounds were tested in a 10-dose IC₅₀ mode fluorescence coupling enzyme assay with 3-fold serial dilution in duplicate starting at 100 μM. The production of FAD-dependent H₂O₂ as a result of demethylase activity of LSD1 on 10 M histone H3₍₁₋₂₁₎K4me2 peptide substrate was measured by coupling with HRP and Amplex Red to yield resorufin (fluorescence measured at Ex/Em=535/590 nm on EnVision, Perkin Elmer).

TABLE 11
LSD1 Data.

| | LSD1 IC₅₀ |
|---|---|
| Ex. No. | (nM) |
| 2 | A |
| 3 | C |
| 4 | A |
| 5 | C |
| 6 | A |
| 7 | A |
| 8 | A |
| 9 | A |
| 10 | B |
| 11 | A |
| 12 | A |
| 13 | C |
| 14 | A |
| 15 | B |
| 16 | A |
| 17 | A |
| 18 | A |
| 19 | A |
| 20 | C |
| 21 | B |
| 22 | B |
| 23 | C |
| 24 | C |
| 25 | C |
| 26 | B |
| 27 | C |
| 28 | B |
| 29 | C |
| 30 | C |
| 31 | D |
| 32 | C |
| 33 | A |
| 34 | B |
| 35 | A |
| 36 | A |
| 37 | B |
| 38 | D |
| 39 | D |
| 40 | A |
| 41 | A |
| 42 | A |
| 43 | A |
| 44 | A |
| 45 | A |
| 46 | A |
| 47 | A |
| 48 | A |
| 49 | A |
| 50 | A |
| 51 | A |
| 53 | C |
| 54 | C |
| 55 | ND |
| 56 | ND |
| 57 | A |
| 58 | A |
| 59 | A |
| 60 | A |
| 61 | A |
| 62 | A |
| 63 | A |
| 64 | A |
| 65 | A |
| 66 | A |
| 67 | A |
| 68 | A |
| 69 | A |
| 70 | A |
| 71 | A |
| 80 | A |
| 84 | A |
| 85 | A |
| 86 | D |
| 87 | D |
| 88 | D |
| 89 | A |
| 90 | A |
| 91 | C |
| 92 | B |
| 93 | A |
| 94 | A |
| 95 | A |
| 96 | ND |

A = <500 nM; B = 500-1000 nM; C = 1000-10000 nM; D = >10000 nM
ND = Not determined

### Determination of IC50 in MV4;11 Cell Line

Cell growth inhibition against MV4;11 cells was confirmed for the synthetic compounds. The Cell Titer-Glo Luminescent Cell Viability Assay (Promega, USA) was used to determine the number of viable cells in culture based on quantitation of the ATP present, which signals the presence of metabolically active cells. This assay was run in a 96-well plate format arrayed in a 10-point dose curve, ranging from 0 to 50 μM with 4-fold dilutions for each compound. Cells are seeded on Day 0 at 3750 cells per well. At Day 5, a portion of each well is assayed for viability according to the Cell Titer-Glo protocol. Briefly, cells/plates were centrifuged at 1100×g for 5 minutes. 100 μL of media was removed from each well and replaced with 100 μL of CellTiter-Glo. The plates were shaken at room temperature for 10 minutes and measured on a luminescence plate reader. Another portion of each well from the Day 5 plate is used to seed a new 96 plate containing fresh compound and media set up in the equivalent 10-point dose curve array in order to continue the cell growth inhibition assay for another 5 days. A final timepoint is collected at Day 10 using the Cell Titer-Glo protocol. Luminescence was detected on an EnVision® Multilabel Plate Reader (Perkin Elmer, Waltham, Mass.) and IC₅₀ determination was made using Graph Pad Prism software.

TABLE 12
Cell Titer Data.

| | Cell | Cell Titer |
|---|---|---|
| Ex. | Titer Glo | Glo IC₅₀ |
| No. | IC₅₀ (5 d) | (10 d) |
| 2 | B | |
| 3 | C | |
| 4 | C | |
| 5 | D | |
| 6 | A | |
| 7 | C | |
| 8 | D | |
| 9 | C | |
| 10 | C | |
| 11 | A | A |
| 12 | A | A |
| 13 | C | |
| 14 | C | |
| 15 | C | |
| 16 | A | |
| 17 | C | |
| 18 | A | A |
| 19 | A | A |
| 20 | C | |
| 21 | C | |
| 22 | C | |
| 23 | C | |
| 24 | C | |
| 25 | C | |
| 26 | C | |
| 27 | C | |
| 28 | C | |
| 29 | D | |
| 30 | ND | ND |
| 31 | D | |
| 32 | C | |
| 33 | A | |
| 34 | C | |
| 35 | A | |
| 36 | A | |
| 37 | C | |
| 38 | C | |
| 39 | D | |
| 40 | A | |
| 41 | C | |
| 42 | B | A |
| 43 | | A |
| 44 | A | A |
| 45 | | A |
| 46 | | A |
| 47 | | A |
| 48 | | A |
| 49 | | |
| 50 | | A |
| 51 | | |
| 53 | | A |
| 54 | | C |
| 55 | ND | |
| 56 | ND | |
| 57 | | C |
| 58 | | A |
| 59 | C | A |
| 60 | B | A |
| 61 | A | A |
| 62 | | A |
| 63 | B | A |
| 64 | A | A |
| 65 | B | A |
| 66 | | A |
| 67 | ND | ND |
| 68 | | A |
| 69 | | A |
| 70 | | A |
| 71 | | A |
| 80 | ND | ND |
| 84 | | A |
| 85 | | A |
| 86 | D | |
| 87 | D | |
| 88 | D | |
| 89 | C | |
| 90 | C | |
| 91 | C | |
| 92 | C | |
| 93 | C | |
| 94 | C | |
| 95 | C | |
| 96 | ND | ND |

A = <500 nM; B = 500-1000 nM; C = 1000-10000 nM; D = >10000 nM
ND = Not determined

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

All references cited in this specification are hereby incorporated by reference. The discussion of the references herein is intended merely to summarize the assertions made by their authors and no admission is made that any reference constitutes prior art relevant to patentability. Applicant reserves the right to challenge the accuracy and pertinence of the cited references.

1. A compound of Formula I: 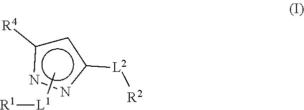
or a salt or tautomer thereof, wherein:
L¹ is chosen from a bond and —(CH₂)ₖ—;
L² is chosen from —C(O)NH—, —NHC(O)—, and —C(O)—;
R¹ is chosen from phenyl, 5-membered monocyclic heteroaryl, and 10-membered bicyclic heteroaryl, either of which is substituted with one R^{5a} and one or more R^{5b};
R² is chosen from cyclohexyl, azetidinyl, pyrrolidinyl, and piperidinyl, any of which is optionally substituted with one or more R⁶;
k is chosen from 1, 2, and 3;
R⁴ is phenyl substituted with one R^{7a} and one or more R^{7b};
R^{5a} is chosen from CH₃, CH₂CH₃, OCH₃, 5- to 6-membered heterocycloalkyl, and 5-membered monocyclic heteroaryl, and 10-membered bicyclic heteroaryl, wherein the heterocycloalkyl and heteroaryl are optionally substituted by one or more R⁹;
each R^{5b} is independently chosen from H, halo, and OCH₃;
each R⁶ is independently chosen from —NHR⁸, —NHC(NH)NHR⁸, and —CH₂NHR⁸;
R^{7a} is cyano;
each R^{7b} is independently chosen from halo;
each R⁸ is independently chosen from H and C₁₋₃ alkyl; and
R⁹ is chosen from cyano, C₁₋₃ alkyl, C₁₋₃ alkoxy, hydroxy, phosphonate, oxo, or any combination thereof,
wherein if R^{5a} is 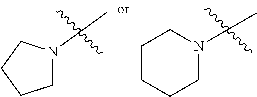 [SMILES: *N1CCCC1 ; *N1CCCCC1]
or optionally substituted by one or more R⁹, each R^{5b} is H.

2. The compound of claim 1, or a salt or tautomer thereof, wherein the compound of Formula I is a compound of Formula III:

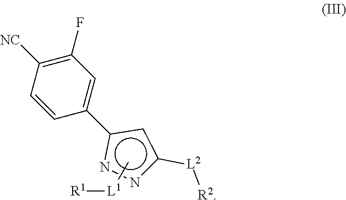

3. The compound of claim 1, or a salt or tautomer thereof, wherein the compound of Formula I is a compound of Formula IV:

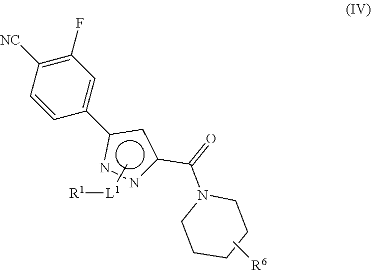

4. The compound of claim 1, or a salt or tautomer thereof, wherein L¹ is —(CH₂)ₖ—.

5. The compound of claim 4, or a salt or tautomer thereof, wherein k is 1 or 2.

6-7. (canceled)

8. The compound of claim 1, or a salt or tautomer thereof, wherein L¹ is a bond.

9. The compound of claim 8, or a salt or tautomer thereof, wherein the compound of Formula I is a compound of Formula V:

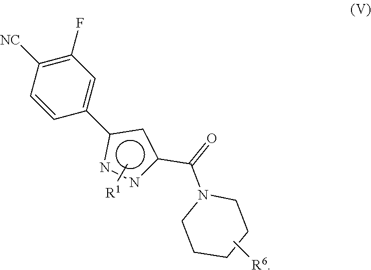

10. The compound of claim 1, or a salt or tautomer thereof, wherein R¹ is phenyl substituted with R^{5a}.

11. The compound of claim 10, or a salt or tautomer thereof, wherein R^{5a} is CH₃ or OCH₃.

12. (canceled)

13. The compound of claim 10, or a salt or tautomer thereof, wherein R¹ is p-tolyl, 3-fluoro-4-methoxyphenyl, or 4-methoxyphenyl.

14. (canceled)

15. The compound of claim 1, or a salt or tautomer thereof, wherein R¹ is monocyclic heteroaryl substituted with R^{5a}.

16. The compound of claim 15, or a salt or tautomer thereof, wherein R¹ is isoxazol-5-yl substituted with R^{5a}.

17. The compound of claim 15, or a salt or tautomer thereof, wherein R^{5a} is CH₃ or CH₂CH₃.

18. The compound of claim 17, or a salt or tautomer thereof, wherein R¹ is 3-methylisoxazol-5-yl or 3-ethylisoxazol-5-yl.

19. The compound of claim 10, or a salt or tautomer thereof, wherein R^{5a} is chosen from pyrrolidin-1-yl, piperidin-1-yl, 4-oxopiperidin-1-yl, piperazin-1-yl, 3-oxopiperazin-1-yl, 1H-indazol-5-yl, 2H-indazol-5-yl, 1H-pyrazol-4-yl, and 1,1-dioxidothiomorpholin-4-yl, any of which is optionally substituted by one, two or three R⁹.

20. The compound of claim 19, or a salt or tautomer thereof, wherein R^{5a} is piperazin-1-yl which is optionally substituted by one R⁹.

21. The compound of claim 20, or a salt or tautomer thereof, wherein R⁹ is C₁₋₃ alkyl.

22. The compound of claim 21, or a salt or tautomer thereof, wherein R⁹ is CH₃.

23. The compound of claim 20, or a salt or tautomer thereof, wherein R^{5a} is 4-methylpiperazin-1-yl.

24. The compound of claim 19, or a salt or tautomer thereof, wherein R^{5a} is piperidin-1-yl optionally substituted by one or two R⁹.

25. The compound of claim 24, or a salt or tautomer thereof, wherein R⁹ is chosen from methoxy, cyano, methyl, hydroxy, —OCH₂CH₂OH, —OCH₂CH₂CH₂OH, and —OCH₂CH₂CH₂OP(O)(OCH₃)₂.

26. The compound of claim 1, or a salt or tautomer thereof, wherein L² is —C(O)NH— or —NHC(O)—.

27. The compound of claim 1, or a salt or tautomer thereof, wherein R² is chosen from cyclohexyl, azetidinyl, pyrrolidin-3-yl, piperidin-3-yl, and piperidin-4-yl, any of which is optionally substituted with R⁶.

28. The compound of claim 27, or a salt or tautomer thereof, wherein R² is cyclohexyl optionally substituted with R⁶.

29. The compound of claim 28, or a salt or tautomer thereof, wherein R⁶ is —NHR⁸.

30. The compound of claim 29, or a salt or tautomer thereof, wherein R⁸ is H.

31. The compound of claim 28, or a salt or tautomer thereof, wherein R² is 3-aminocyclohexyl.

32. The compound of claim 27, or a salt or tautomer thereof, wherein R² is pyrrolidin-3-yl, optionally substituted with R⁶.

33. The compound of claim 32, or a salt or tautomer thereof, wherein R⁶ is —NHR⁸.

34. The compound of claim 33, or a salt or tautomer thereof, wherein R⁸ is H.

35. The compound of claim 32, or a salt or tautomer thereof, wherein R² is pyrrolidin-3-yl.

36. The compound of or a salt or tautomer thereof, wherein L² is —C(O)—.

37. The compound of claim 1, or a salt or tautomer thereof, wherein R² is chosen from azetidinyl and piperidin-1-yl, either of which is optionally substituted with R⁶

38. The compound of claim 37, or a salt or tautomer thereof, wherein R² is piperidin-1-yl optionally substituted with R⁶.

39. The compound of claim 38, or a salt or tautomer thereof, wherein R⁶ is —NHR⁸.

40. The compound of claim 39, or a salt or tautomer thereof, wherein R⁸ is H.

41. The compound of claim 38, or a salt or tautomer thereof, wherein R² is 3-aminopiperidin-1-yl.

42. The compound of claim 37, or a salt or tautomer thereof, wherein R² is azetidin-1-yl optionally substituted with R⁶.

43. The compound of claim 42, or a salt or tautomer thereof, wherein R⁶ is chosen from —CH₂NHR⁸ and —NHR⁸.

44. The compound of claim 43, or a salt or tautomer thereof, wherein R⁸ is H.

45. The compound of claim 1, or a salt or tautomer thereof, wherein R⁴ is phenyl substituted with one R^{7a} and substituted with one R^{7b}.

46. The compound of claim 45, or a salt or tautomer thereof, wherein R⁴ is 3-fluoro-4-cyanophenyl or 4-cyanophenyl.

47. The compound of claim 51, or a salt or tautomer thereof, wherein R⁴ is 3-fluoro-4-cyanophenyl.

48. The compound of claim 1, or a salt or tautomer thereof, wherein the compound of Formula I is chosen from:

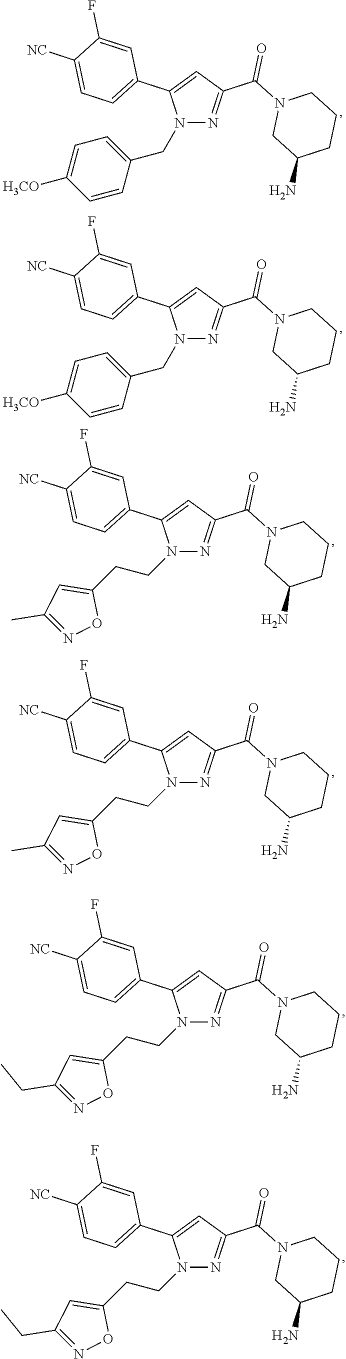

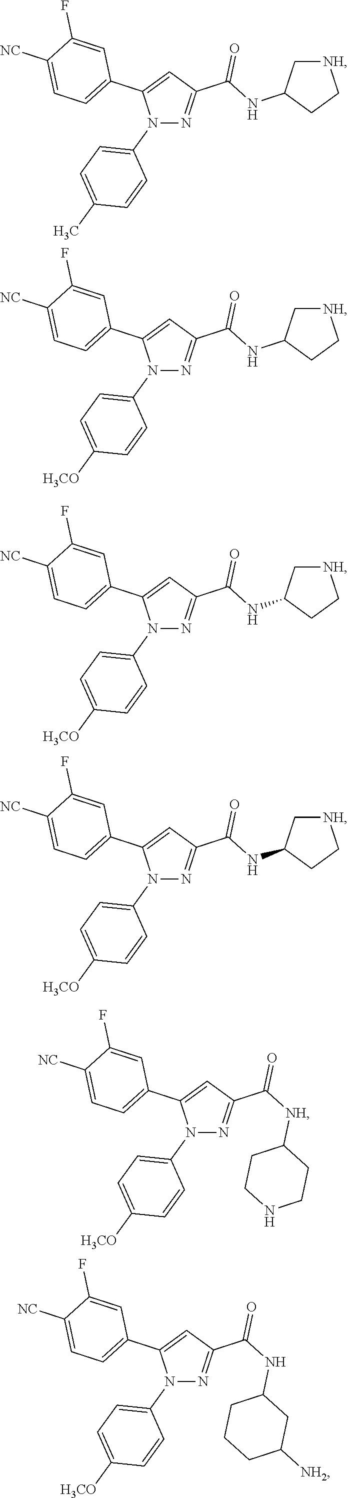

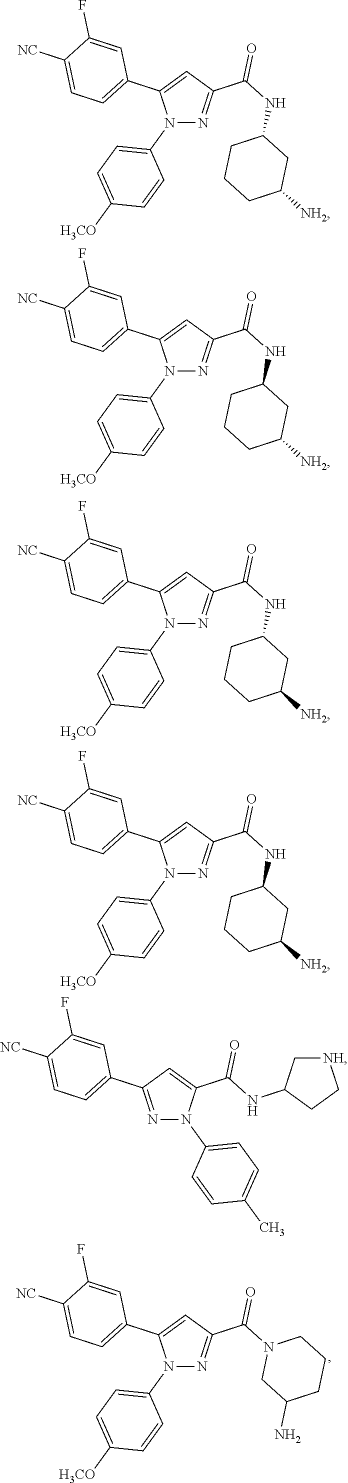

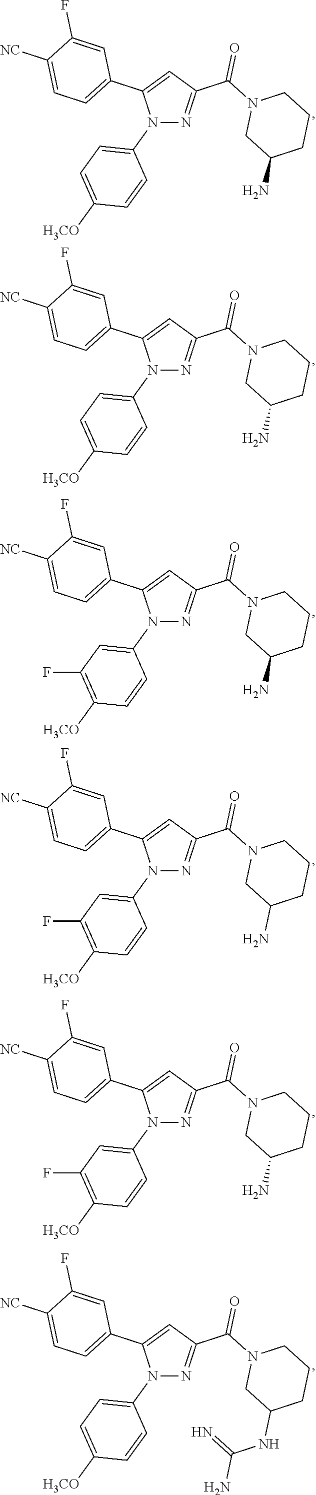

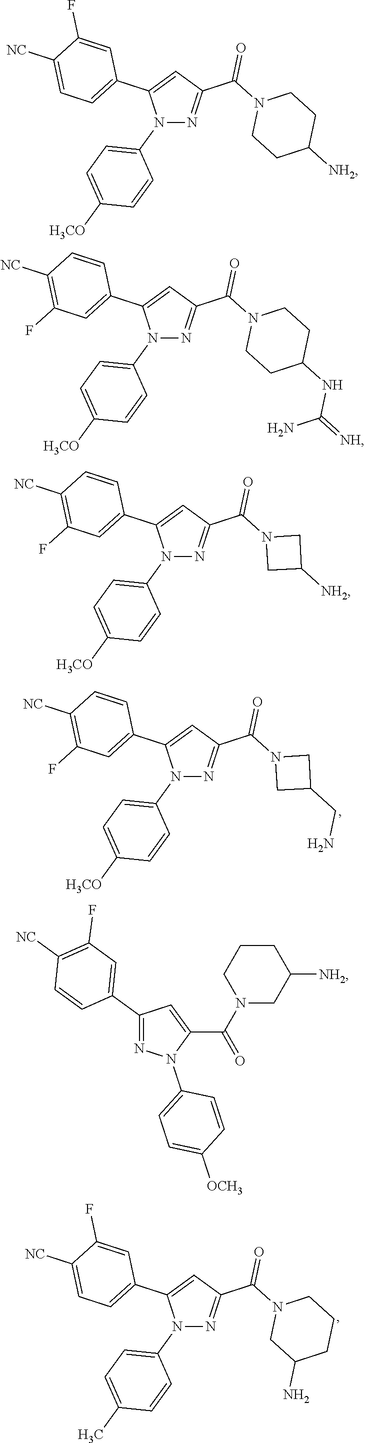

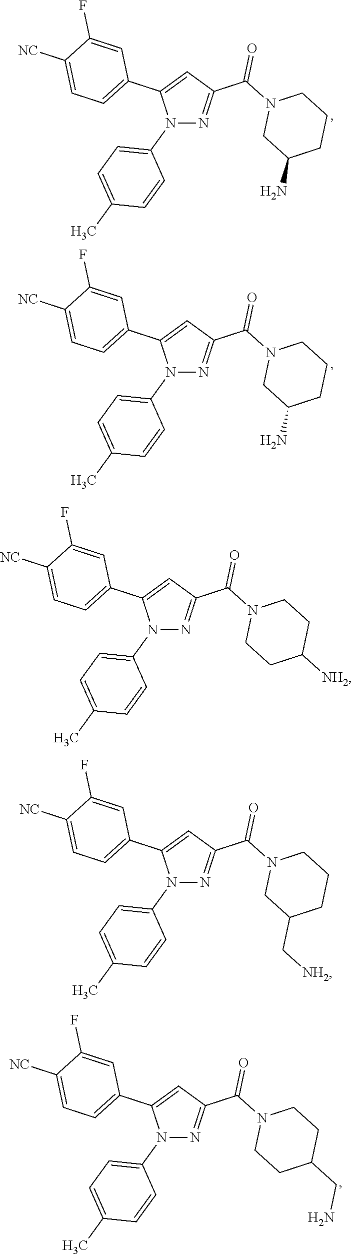

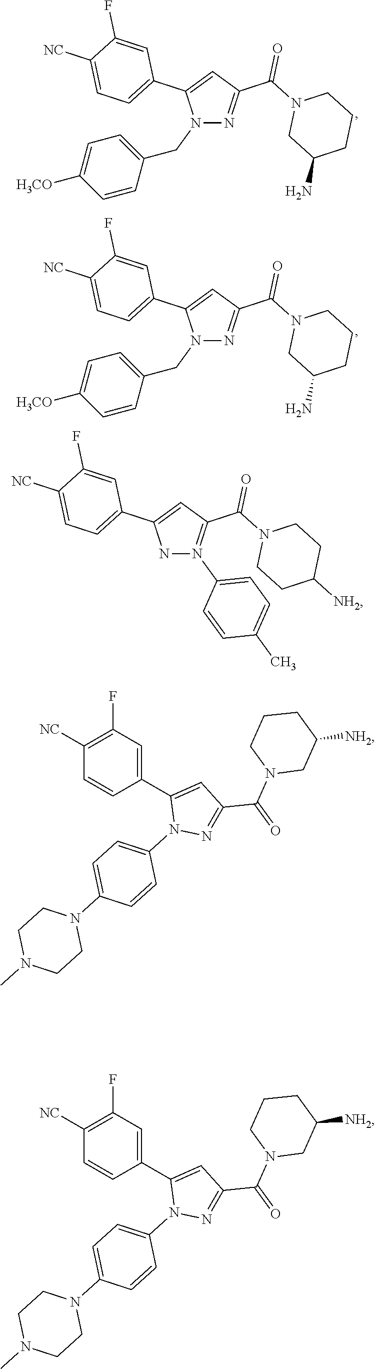

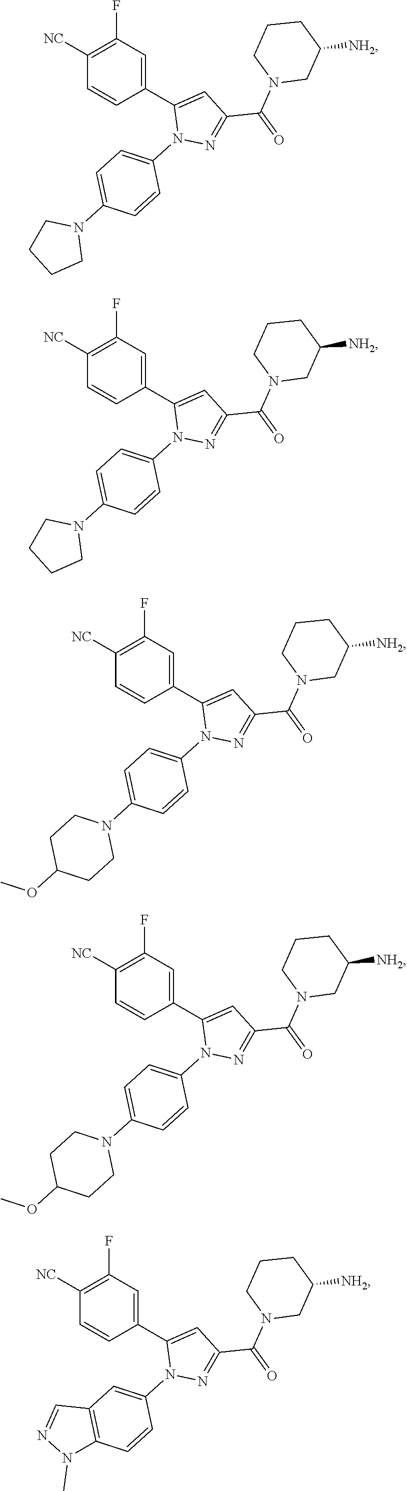 [SMILES: N#Cc1ccc(-c2cc(C(=O)N3CCC[C@H](N)C3)nn2-c2ccc(N3CCCC3)cc2)cc1F ; N#Cc1ccc(-c2cc(C(=O)N3CCC[C@@H](N)C3)nn2-c2ccc(N3CCCC3)cc2)cc1F ; COC1CCN(c2ccc(-n3nc(C(=O)N4CCC[C@H](N)C4)cc3-c3ccc(C#N)c(F)c3)cc2)CC1 ; COC1CCN(c2ccc(-n3nc(C(=O)N4CCC[C@@H](N)C4)cc3-c3ccc(C#N)c(F)c3)cc2)CC1 ; Cn1ncc2cc(-n3nc(C(=O)N4CCC[C@H](N)C4)cc3-c3ccc(C#N)c(F)c3)ccc21]

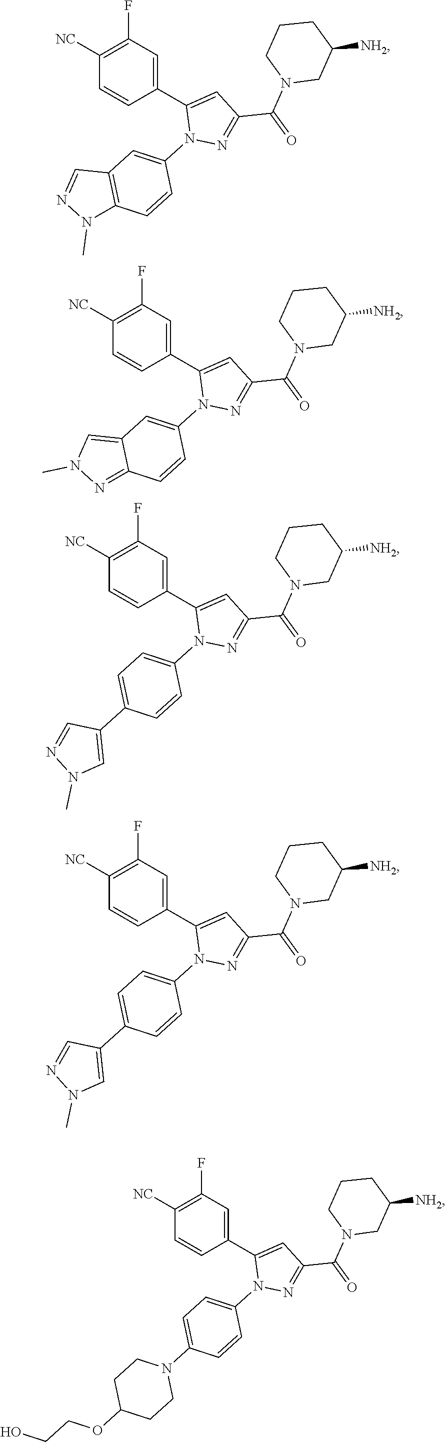

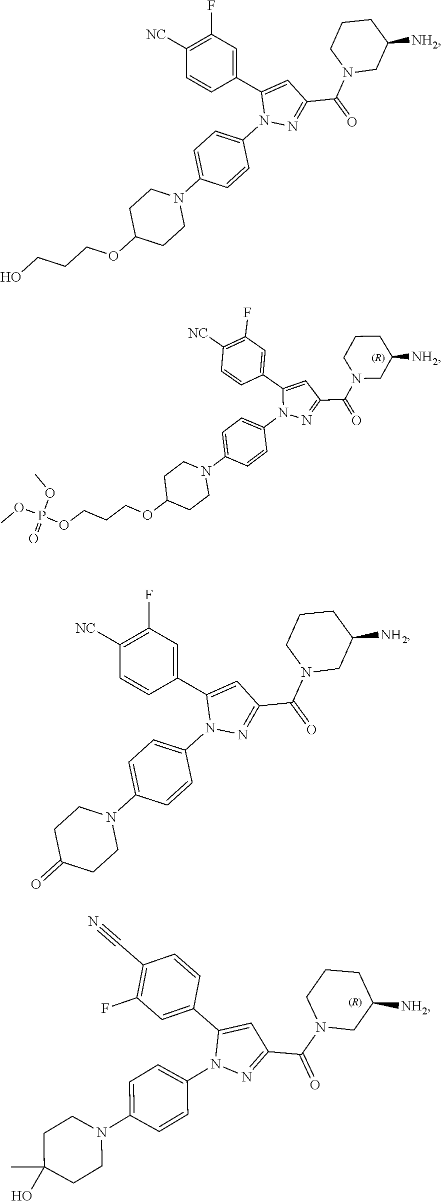

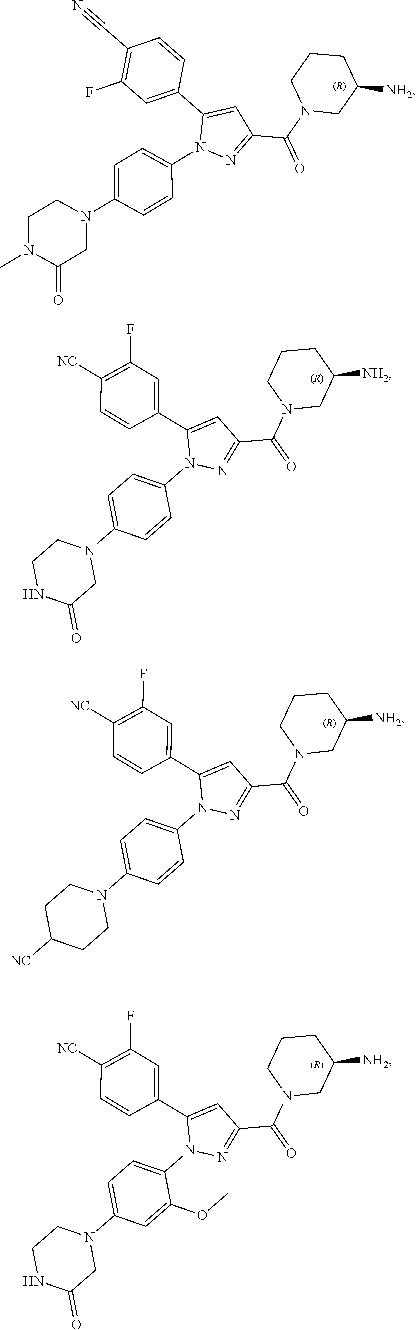

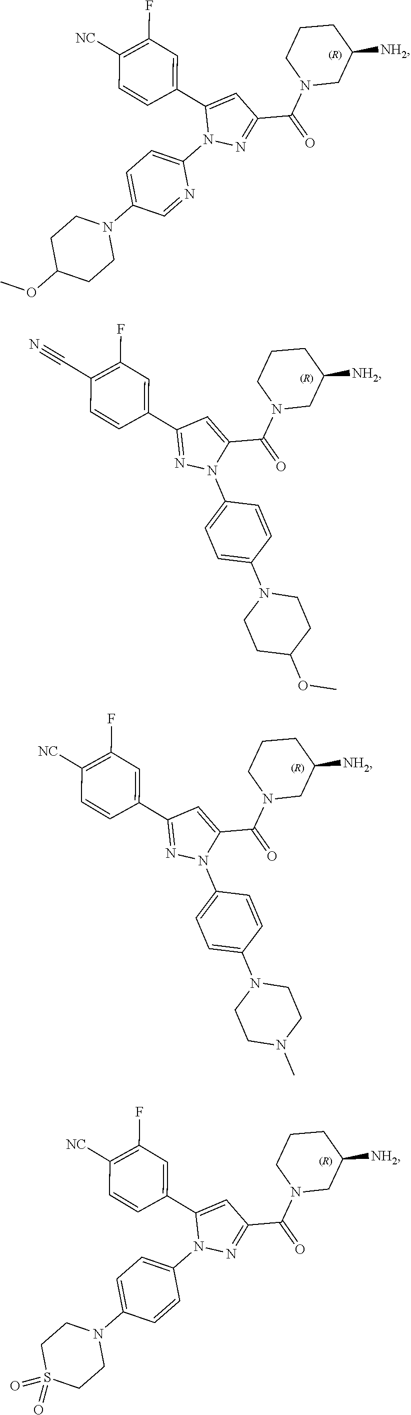

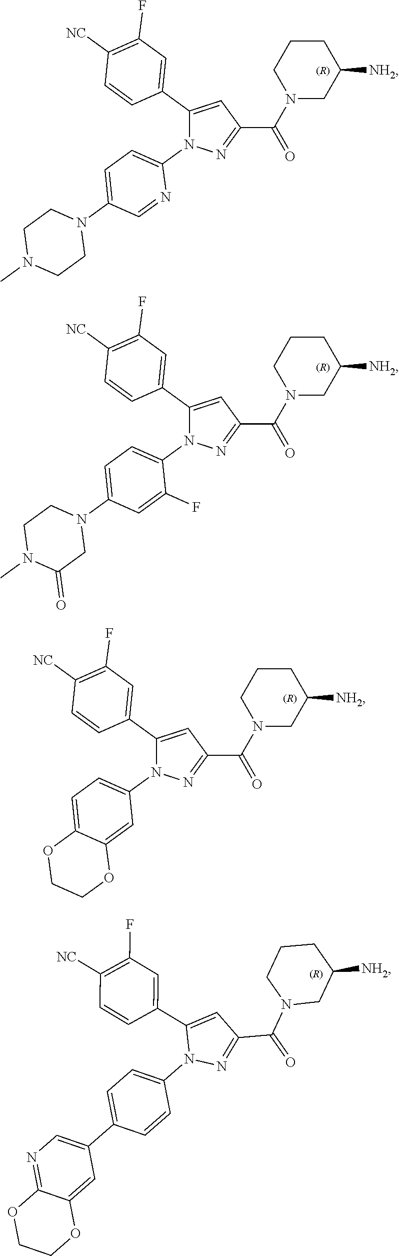

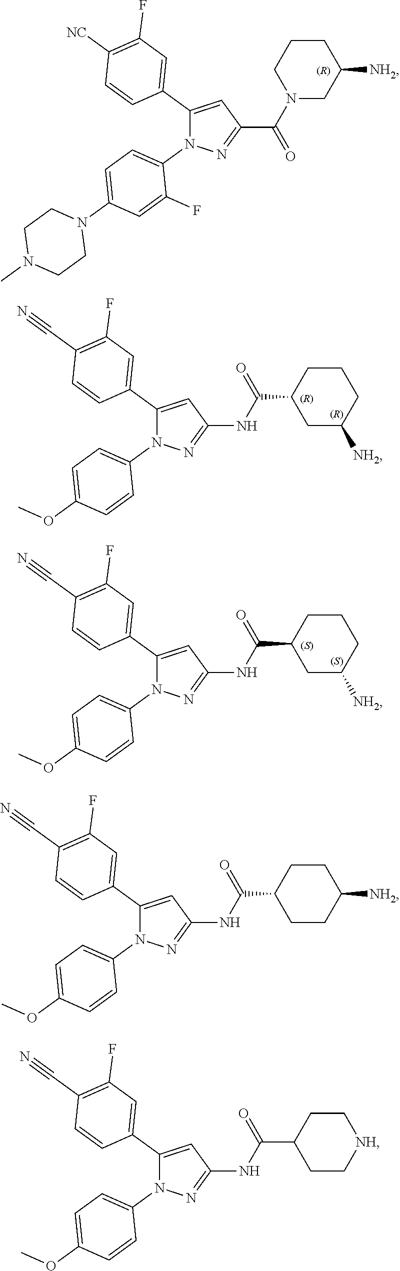

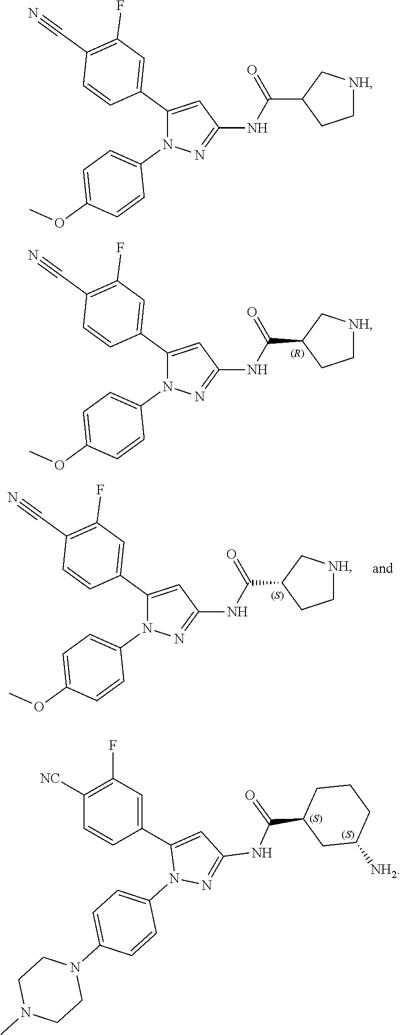

49. A compound of Formula I or II: 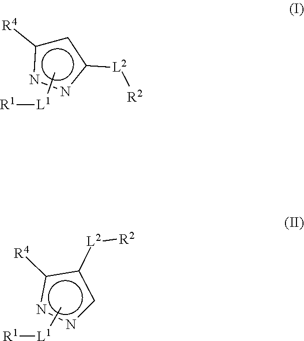
or a salt or tautomer thereof, wherein:
L¹ is a bond;
L² is chosen from —C(O)NH—, —NHC(O)—, and —C(O)—;
R¹ is phenyl substituted with R^{5a};
R² is chosen from cyclohexyl, azetidinyl, pyrrolidinyl, and piperidinyl, any of which is optionally substituted with one or more R⁶;
R⁴ is phenyl which is substituted with one R^{7a} and optionally substituted with one or more R^{7b};
R^{5a} is OCH₃;
each R⁶ is independently chosen from —NHR⁸, —NHC(NH)NHR⁸, and —CH₂NHR⁸;
R^{7a} is cyano;
each R^{7b} is independently chosen from halo; and
each R⁸ is independently chosen from H and C₁₋₃ alkyl.

50. The compound of claim 49, or a salt or tautomer thereof, wherein L² is —C(O)—.

51. The compound of claim 49, or a salt or tautomer thereof, wherein L² is —C(O)NH— or —NHC(O)—.

52. The compound of claim 49, or a salt or tautomer thereof, wherein R¹ is 4-methoxyphenyl.

53. The compound of claim 49, or a salt or tautomer thereof, wherein R² is chosen from piperidin-1-yl and piperidin-3-yl, either of which is optionally substituted with one or more R⁶.

54. The compound of claim 53, or a salt or tautomer thereof, wherein R² is piperidin-1-yl, optionally substituted with R⁶.

55. The compound of claim 54, or a salt or tautomer thereof, wherein R⁶ is —NHR⁸.

56. The compound of claim 55, or a salt or tautomer thereof, wherein R⁸ is H.

57. The compound of claim 53, or a salt or tautomer thereof, wherein R² is 3-aminopiperidin-1-yl.

58. The compound of claim 53, or a salt or tautomer thereof, wherein R² is piperidin-3-yl, optionally substituted with one or more R⁶.

59. The compound of claim 58, or a salt or tautomer thereof, wherein R⁶ is chosen from —NHR⁸ and —CH₂NHR⁸.

60. The compound of claim 59, or a salt or tautomer thereof, wherein R⁸ is H.

61. The compound of claim 58, or a salt or tautomer thereof, wherein R² is piperidin-3-yl.

62. The compound of claim 1, or a salt or tautomer thereof, wherein R⁴ is phenyl substituted with one R^{7a}.

63. The compound of claim 62, or a salt or tautomer thereof, wherein R⁴ is 4-cyanophenyl.

64. The compound of claim 49, or a salt or tautomer thereof, wherein the compound of Formula I is chosen from:

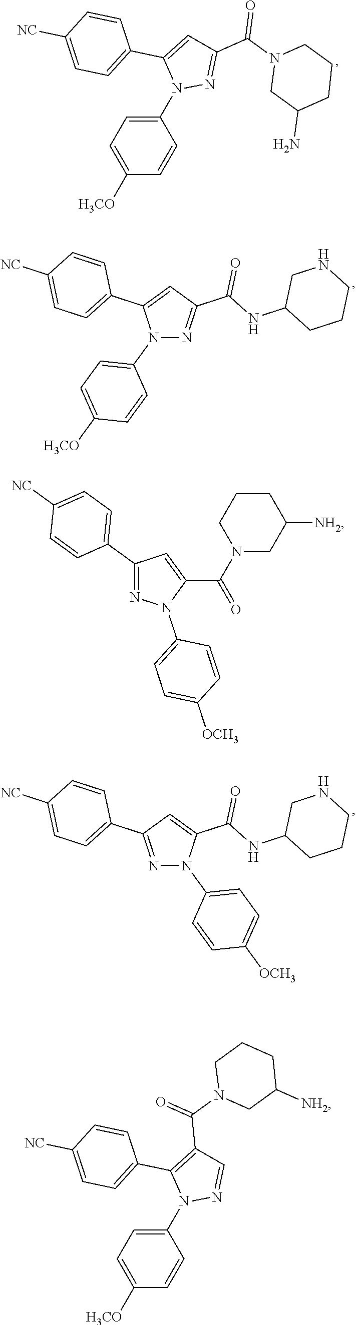 [SMILES: COc1ccc(-n2nc(C(=O)N3CCCC(N)C3)cc2-c2ccc(C#N)cc2)cc1 ; COc1ccc(-n2nc(C(=O)NC3CCCNC3)cc2-c2ccc(C#N)cc2)cc1 ; COc1ccc(-n2nc(-c3ccc(C#N)cc3)cc2C(=O)N2CCCC(N)C2)cc1 ; COc1ccc(-n2nc(-c3ccc(C#N)cc3)cc2C(=O)NC2CCCNC2)cc1 ; COc1ccc(-n2ncc(C(=O)N3CCCC(N)C3)c2-c2ccc(C#N)cc2)cc1]

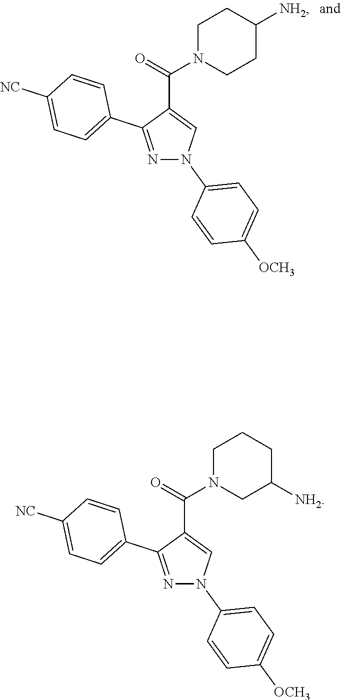 [SMILES: COc1ccc(-n2cc(C(=O)N3CCC(N)CC3)c(-c3ccc(C#N)cc3)n2)cc1 ; COc1ccc(-n2cc(C(=O)N3CCCC(N)C3)c(-c3ccc(C#N)cc3)n2)cc1]

65. A pharmaceutical composition comprising a compound as recited in ai, or a salt or tautomer thereof, together with a pharmaceutically acceptable carrier.

66. The pharmaceutical composition as recited in claim 65, formulated for oral administration.

67. The pharmaceutical composition as recited in claim 65, additionally comprising another therapeutic agent.

68. A method of inhibition of KDM1A comprising contacting KDM1A with a compound as recited in claim 1, or a salt or tautomer thereof.

69. A method of treatment of a KDM1A-mediated disease comprising the administration of a therapeutically effective amount of a compound as recited in claim 1, to a patient in need thereof.

70-75. (canceled)

76. A method of treatment of a bone marrow failure syndrome comprising the administration of a therapeutically effective amount of a compound as recited in claim 1, or a salt or tautomer thereof.

77-78. (canceled)

79. A method of treatment of a disease resulting from mutations in globin genes which affect expression or function of the globin protein, comprising the administration of a therapeutically effective amount of a compound as recited in claim 1, or a salt or tautomer thereof, to a patient in need thereof.

80. A method for achieving an effect in a patient, comprising the administration of a therapeutically effective amount of a compound as recited in claim 1, or a salt or tautomer thereof, to a patient, wherein the effect is chosen from an elevation of red blood cell count, an elevation of the red blood cell count of red cells containing fetal hemoglobin, an elevation in the total concentration of fetal hemoglobin in red cells, an elevation in the total concentration of fetal hemoglobin in reticulocytes, an increase in the transcription of the gamma globin gene in bone marrow-derived red cell precursors, a reduction in the number of sickle cell crises a patient experiences over a unit period of time, a halt to or prevention of tissue damage caused by sickling cells, a reduction in the proportion of red cells that undergo sickling under physiological conditions of relative hypoxia as measured using patient blood in an in vitro assay, an increase in the amount of histone 3 lysine methylation at lysine position 4, and/or a decrease in the amount of histone 3 methylation at lysine position 9 near or at the gamma globin promoter as assayed by ChIP using cells derived from a treated patient.

81. A method of treatment of a hemoglobinopathy, comprising the administration of a therapeutically effective amount of a compound as recited in claim 1, or a salt or tautomer thereof, to a patient in need thereof.

82-85. (canceled)

86. A method of inhibiting at least one KDM1A function comprising the step of contacting KDM1A with a compound as recited in claim 1, or a salt or tautomer thereof, wherein the inhibition is measured by phenotype of red cells or their precursors either cultured or in vivo in humans or mouse or transgenic mice containing the human beta globin locus or portions thereof, the ability of cancer cells to proliferate, become differentiated, or induced to undergo apoptosis, the expression of specific genes known to be regulated by KDM1A activity, a change in the histone methylation states, a change in the methylation state of proteins known to be demethylated by KDM1A, expression of KDM1A-regulated genes, or binding of KDM1A with a natural binding partner.
